SEQUENCE LISTING (51 sequences)

SEQ ID NO: 1 - DNA, 613 bp, Artificial Sequence
  Description of Artificial Sequence Synthetic polynucleotide
gtacactggcacggggtgtgccccgggcactgactgtaccacagccataggctgggcttcagccagccccagggcctgaacctgggccttcactctctctgcctcctcgggcaggatgtccaggcaggcagccttgaggagggtgatctggtcggcgatggtgagggtggtgaagccgggcagctgcttggcgaactccacagtcttaatgatgcacttggtggagagttcactgaacttgtcccagaggtcaatgtccagagagacacgttgttctgagctgttgttcgtagtgtatttgcccagctggcagagggcagggaaggtttcctggtgcgctttgcgcaccttctcaatgagctcccccacctccggcgtcagcgtgtagctctcagagcactcgggcttgggcacctccttcttcttcttgtttcggtcgtttctcacagactccttggacatgcccacttcaaagcacttctgcagtcggcagtactggcagcggttccgggtcaccttgttgatgatgcagttcttgtcccggtgacacgtgtacaccatgttcttctggatgctgcggcggaagaagcccttgcagccctcacaggcgctg

SEQ ID NO: 2 - DNA, 277 bp, Artificial Sequence
  Description of Artificial Sequence Synthetic polynucleotide
gtacactggcacggggtgtgccccgggcactgactgtaccacagccataggctgggcttcagccagccccagggcctgaacctgggccttcactctctctgcctcctcgggcaggtcaacgtcaatagggtccctgggagtgctggcagcggttccgggtcaccttgttgatgatgcagttcttgtcccggtgacacgtgtacaccatgttcttctggatgctgcggcggaagaagcccttgcagccctcacaggcgctgaccccatagtggtagcc

SEQ ID NO: 3 - DNA, 240 bp, Artificial Sequence
  Description of Artificial Sequence Synthetic polynucleotide
  modified_base (3)..(3): a, c, t, g, unknown or other
  modified_base (9)..(9): a, c, t, g, unknown or other
  modified_base (12)..(12): a, c, t, g, unknown or other
ctnttggantcnggcccaggggccgagacattcccttcttcactcttttcctcgacgggctttcgtggaggaggctcactactgcttttctttggggcaggatcctctcttgctgatggggtaggtttctgactagagtccaccacgttcttcacaacactgctctctttgctgtctttcttttcactggtcttagactttttctctttctttttcacagcttgttgcctggtctgggat

SEQ ID NO: 4 - DNA, 535 bp, Artificial Sequence
  Description of Artificial Sequence Synthetic polynucleotide
  modified_base (7)..(7): a, c, t, g, unknown or other
ggattcnggcccaggggccgagacattcccttcttcactcttttcctcgacgggctttcgtggaggaggctcactactgcttttctttggggcaggatcctctcttgctgatggggtaggtttctgactagagtccaccacgttcttcacaacactgctctctttgctgtctttcttttcactggtcttagactttttctctttctttttcacagctttagcttgcttctgcaggtaggctttggaaggcatccattgtagattctgacattttctcatcttgcagcactgcttctttatattgcgaccaccaaacttgggcttatctaagcaattagtacaaacaccacagtcctcaggcacctggcagccgggacactgcccacaccgcctcgatcgacgtcctttctttactggaggttcctggggtgccttgtttctagtgacaggtttaattggtttgatgggtggagcaagaggttcagcatcttctgagccagcaattgatgacttgtcttgttgcctggtctgggat

SEQ ID NO: 5 - DNA, 287 bp, Artificial Sequence
  Description of Artificial Sequence Synthetic polynucleotide
atgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatctattcctctacacagccgactagttatgatcagagcagttactctcagcagaacacctatgggcaaccgagcagctatggacagcagagtagctatggtcaacaaagcagctatgggcagcagcctcccactagtta

SEQ ID NO: 6 - DNA, 325 bp, Artificial Sequence
  Description of Artificial Sequence Synthetic polynucleotide
ggcgagctactatagaaagggaggctgtgccatgctgccagttaagtggatgcccccagaggccttcatggaaggaatattcacttctaaaacagacacatgacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgctta

SEQ ID NO: 7 - DNA, 16 bp, Artificial Sequence
  Description of Artificial Sequence Synthetic oligonucleotide
tcctcgggcaggatgt

SEQ ID NO: 8 - DNA, 16 bp, Artificial Sequence
  Description of Artificial Sequence Synthetic oligonucleotide
ctgggagtgctggcag

SEQ ID NO: 9 - DNA, 14 bp, Artificial Sequence
  Description of Artificial Sequence Synthetic oligonucleotide
gacttgtcttgttg

SEQ ID NO: 10 - DNA, 14 bp, Artificial Sequence
  Description of Artificial Sequence Synthetic oligonucleotide
tttcacagcttgtt

SEQ ID NO: 11 - DNA, 22 bp, Artificial Sequence
  Description of Artificial Sequence Synthetic primer
atgcagctgtatccaagaaagc

SEQ ID NO: 12 - DNA, 19 bp, Artificial Sequence
  Description of Artificial Sequence Synthetic primer
ctccgcagatgaggcagat

SEQ ID NO: 13 - DNA, 20 bp, Artificial Sequence
  Description of Artificial Sequence Synthetic primer
gctaccactatggggtcagc

SEQ ID NO: 14 - DNA, 23 bp, Artificial Sequence
  Description of Artificial Sequence Synthetic primer
cgttgtattggagacatcctctc

SEQ ID NO: 15 - DNA, 20 bp, Artificial Sequence
  Description of Artificial Sequence Synthetic primer
gtgtcaccgggacaagaact

SEQ ID NO: 16 - DNA, 20 bp, Artificial Sequence
  Description of Artificial Sequence Synthetic primer
ctgggccttcactctctctg

SEQ ID NO: 17 - DNA, 21 bp, Artificial Sequence
  Description of Artificial Sequence Synthetic primer
gtgttgtgaagaacgtggtgg

SEQ ID NO: 18 - DNA, 21 bp, Artificial Sequence
  Description of Artificial Sequence Synthetic primer
cggctgcctcctctatttaca

SEQ ID NO: 19 - DNA, 20 bp, Artificial Sequence
  Description of Artificial Sequence Synthetic primer
gaggaccccggattaaacat

SEQ ID NO: 20 - DNA, 20 bp, Artificial Sequence
  Description of Artificial Sequence Synthetic primer
tggtggaggttcgtgatgta

SEQ ID NO: 21 - DNA, 20 bp, Artificial Sequence
  Description of Artificial Sequence Synthetic primer
tctgaacaacccagtcctgc

SEQ ID NO: 22 - DNA, 20 bp, Artificial Sequence
  Description of Artificial Sequence Synthetic primer
tggtggaggctgctttttct

SEQ ID NO: 23 - DNA, 22 bp, Artificial Sequence
  Description of Artificial Sequence Synthetic primer
ccaggcaacaagacaagtcatc

SEQ ID NO: 24 - DNA, 23 bp, Artificial Sequence
  Description of Artificial Sequence Synthetic primer
gcttctttatattgcgaccacca

SEQ ID NO: 25 - DNA, 20 bp, Artificial Sequence
  Description of Artificial Sequence Synthetic primer
atcctacagccaagctccaa

SEQ ID NO: 26 - DNA, 20 bp, Artificial Sequence
  Description of Artificial Sequence Synthetic primer
ggacttttgttgaggccaga

SEQ ID NO: 27 - DNA, 20 bp, Artificial Sequence
  Description of Artificial Sequence Synthetic primer
tggcctcaacaaaagtcctc

SEQ ID NO: 28 - DNA, 20 bp, Artificial Sequence
  Description of Artificial Sequence Synthetic primer
ttggagcttggctgtaggat

SEQ ID NO: 29 - DNA, 20 bp, Artificial Sequence
  Description of Artificial Sequence Synthetic primer
gcagagccctgagtacaagc

SEQ ID NO: 30 - DNA, 20 bp, Artificial Sequence
  Description of Artificial Sequence Synthetic primer
ccacagccaaaacaacttca

SEQ ID NO: 31 - DNA, 20 bp, Artificial Sequence
  Description of Artificial Sequence Synthetic primer
cacacctgggaaaggaccta

SEQ ID NO: 32 - DNA, 20 bp, Artificial Sequence
  Description of Artificial Sequence Synthetic primer
tgccagcaaagcagtagttg

SEQ ID NO: 33 - DNA, 19 bp, Artificial Sequence
  Description of Artificial Sequence Synthetic primer
ccagagcagagcaaacaga

SEQ ID NO: 34 - DNA, 17 bp, Artificial Sequence
  Description of Artificial Sequence Synthetic primer
tttcacagcttgttgcc

SEQ ID NO: 35 - DNA, 18 bp, Artificial Sequence
  Description of Artificial Sequence Synthetic primer
cccaagaaaaagcagcct

SEQ ID NO: 36 - DNA, 19 bp, Artificial Sequence
  Description of Artificial Sequence Synthetic primer
tggtctgggatggtgtgaa

SEQ ID NO: 37 - DNA, 35 bp, Artificial Sequence
  Description of Artificial Sequence Synthetic primer
acacgcggccgctttatttatttgtttattgagac

SEQ ID NO: 38 - DNA, 31 bp, Artificial Sequence
  Description of Artificial Sequence Synthetic primer
acacgaattcggccaggtgcagtggtggctc

SEQ ID NO: 39 - DNA, 30 bp, Artificial Sequence
  Description of Artificial Sequence Synthetic primer
acacgaattctatggtggctctgtaattct

SEQ ID NO: 40 - DNA, 20 bp, Artificial Sequence
  Description of Artificial Sequence Synthetic primer
cttgggctcactaggagtgg

SEQ ID NO: 41 - DNA, 20 bp, Artificial Sequence
  Description of Artificial Sequence Synthetic primer
agtctaagaccagtgaaaag

SEQ ID NO: 42 - DNA, 20 bp, Artificial Sequence
  Description of Artificial Sequence Synthetic primer
cttgttgcctggtctgggat

SEQ ID NO: 43 - DNA, 20 bp, Artificial Sequence
  Description of Artificial Sequence Synthetic primer
acccagtcctgccagctcca

SEQ ID NO: 44 - DNA, 34 bp, Artificial Sequence
  Description of Artificial Sequence Synthetic primer
acacctcgaggataaatcaagacattaagatggt

SEQ ID NO: 45 - DNA, 38 bp, Artificial Sequence
  Description of Artificial Sequence Synthetic primer
gctcggtacccggggatccttgggcagtgtgattcctg

SEQ ID NO: 46 - DNA, 33 bp, Artificial Sequence
  Description of Artificial Sequence Synthetic primer
ctgcctcctcgggctgcaaagttgggaaaattg

SEQ ID NO: 47 - DNA, 28 bp, Artificial Sequence
  Description of Artificial Sequence Synthetic primer
aactttgcagcccgaggaggcagagaga

SEQ ID NO: 48 - DNA, 28 bp, Artificial Sequence
  Description of Artificial Sequence Synthetic primer
agaccctcaccaggatgtccaggcaggc

SEQ ID NO: 49 - DNA, 28 bp, Artificial Sequence
  Description of Artificial Sequence Synthetic primer
ggacatcctggtgagggtctgcaccctg

SEQ ID NO: 50 - DNA, 38 bp, Artificial Sequence
  Description of Artificial Sequence Synthetic primer
tgcatgcctgcaggtcgactggatgggagcccctgcag

SEQ ID NO: 51 - DNA, 39 bp, Artificial Sequence
  Description of Artificial Sequence Synthetic oligonucleotide
aagcaagctaaagctgtgaaaaagaaagagaaaaagtct

(19) United States
(12) Patent Application Publication
(10) Pub. No.: US 2017/0298347 A1
(43) Pub. Date: Oct. 19, 2017

(54) NOVEL FUSION-CIRCULAR RNAs AND USES THEREOF

(71) Applicant: BETH ISRAEL DEACONESS MEDICAL CENTER, Boston, MA (US)

(72) Inventors: Pier Paolo Pandolfi, Brookline, MA (US); Jlenia Guarnerio, Cambridge, MA (US)

(21) Appl. No.: 15/417,868

(22) Filed: Jan. 27, 2017

Related U.S. Application Data
(60) Provisional application No. 62/290,679, filed on Feb. 3, 2016.

(51) Int. Cl. C12N 15/11; C12Q 1/68; C12N 5/073; C12N 15/113

(52) U.S. Cl. CPC C12N 15/11; C12N 15/1135; C12Q 1/6886; C12N 5/0603; C12N 2320/33; C12N 2310/531; C12N 2310/14; C12N 2310/113; C12N 2501/00; C12Q 2600/158; C12Q 2600/178; C12N 2310/532

(57) ABSTRACT

Novel fusion-circular RNAs (f-circRNAs) and complements thereof are provided. Diagnostic and treatment methods using f-circRNA inhibitors are provided. Non-human animals expressing exogenous f-circRNA and complements thereof are also provided.

### CROSS-REFERENCE TO RELATED APPLICATION

This application claims the benefit of priority of U.S. Provisional Patent Application Ser. No. 62/290,679, filed Feb. 3, 2016, the entire disclosure of which is hereby incorporated herein by reference.

### SEQUENCE LISTING

The instant application contains a Sequence Listing which has been submitted electronically in ASCII format and is hereby incorporated by reference in its entirety. Said ASCII copy, created on Mar. 20, 2017, is named 587946BI9-001_SL.txt and is 14,889 bytes in size.

### FIELD OF THE INVENTION

This disclosure relates to novel fusion-circular RNAs (f-circRNAs) and complements thereof. This disclosure also relates to the uses of f-circRNAs, e.g., for the diagnosis and/or treatment of translocation-associated disorders.

### BACKGROUND

Recurrent genomic alterations such as point mutations, chromosomal deletions, amplifications and translocations have been historically associated with the pathogenesis of several diseases, including cancer (Chin et al., 2011; Rabbitts, 1994; Meyerson et al., 2010). The role of genomic alterations in the process of tumorigenesis is attributed to their capacity to encode new oncogenic proteins. Such proteins are generally mutated versions of functional cellular proteins and are often the primary cause of the onset and progression of cancer. For instance, aberrant chromosomal translocations result in the rearrangement of parts of non-homologous chromosomes, joining together two otherwise separated genes. Therefore, the down-stream gene falls under the promoter of the upstream gene and their transcription forms a new functional mRNA that is translated into a protein made from elements of both genes. Such proteins are also labeled as oncogenic “fusion proteins” (Somervaille and Cleary, 2010; Greuber et al., 2013).

Although oncogenic proteins are undoubtedly important factors in cancer development, there is mounting evidence demonstrating that proteins are not the only entities involved in disease pathogenesis. The non-coding dimension, which includes micro-RNAs (miRNAs) (Croce, 2009; Ryan et al., 2010), pseudogenes (Karreth et al., 2015), long-non-coding-RNAs (lncRNAs) (Cheetham et al., 2013) and the recently discovered circular RNAs (circRNAs) (Jeck et al., 2013; Li et al., 2015a; Qu et al., 2015), is becoming more and more relevant in the processes of tumor onset and progression. Whether or not genomic alterations could impact the non-coding RNA dimension has been so far only poorly investigated (Calin and Croce, 2007) and their impact on circRNAs is completely unexplored.

### SUMMARY

The present invention in based in part on the discovery of novel circRNAs termed fusion-circRNAs (f-circRNAs) that comprise chromosomal translocations. It has been surprisingly discovered that f-circRNAs are functionally relevant and tumor promoting, with important diagnostic and therapeutic implications as described further herein.

Accordingly, in one aspect, provided herein is an isolated f-circRNA or a complement thereof encoding one or more exons or exon fragments from a first gene, one or more exons or exon fragments from a second gene and an f-circRNA back-splice junction. In an embodiment, the first and second genes are arranged as a translocation that corresponds to a disorder selected from the group consisting of cancer, translocation Down syndrome, XX male syndrome and schizophrenia. In another embodiment, the first and second genes are arranged as a translocation selected from the group consisting of a balanced translocation, an unbalanced translocation, a Robertsonian translocation and an insertional translocation. In still another embodiment, the first and second genes correspond to a cancer-associated chromosomal translocation. In a specific embodiment, the cancer-associated chromosomal translocation is selected from the group consisting of a PML/RARα translocation, an MLL/AF9 translocation, an EWSR1-FL11 translocation and an AML4/ALK1 translocation. In another embodiment, the PML/RARα translocation comprises a back-splice junction between a 5′ head of PML exon 5 and a 3′ tail of RARα exon 6. In another embodiment, the PML/RARα translocation comprises a back-splice junction between a 5′ head of PML exon 4 and a 3′ tail of RARα exon 4. In another embodiment, the MLL/AF9 translocation comprises a back-splice junction between a 5′ head of MLL exon 7 and a 3′ tail of AF9 exon 6. In an embodiment, the MLL/AF9 translocation comprises a back-splice junction between a 5′ head of MLL exon 5 and a 3′ tail of AF9 exon 6. In another embodiment, the EWSR1/FLI1 translocation comprises a back-splice junction between a 5′ head of EWSR1 exon 7 and a 3′ tail of FLI1 exon 10. In another embodiment, the EML4/ALK1 translocation comprises a back-splice junction between a 5′ head of EML4 exon 12 and a 3′ tail of ALK1 exon 26. In another embodiment, the f-circRNA comprises a nucleotide sequence having at least 80% homology to a sequence selected from the group consisting of SEQ ID NO:1, SEQ ID NO:2, SEQ ID NO:3, SEQ ID NO:4, SEQ ID NO:5 and SEQ ID NO:6. In another embodiment, the f-circRNA comprises a nucleotide sequence selected from the group consisting of SEQ ID NO:1, SEQ ID NO:2, SEQ ID NO:3, SEQ ID NO:4, SEQ ID NO:5 and SEQ ID NO:6.

In another aspect, provided herein is a vector expressing an f-circRNA or a complement thereof.

In another aspect, provided herein is a compound that binds to an f-circRNA back-splice junction of an f-circRNA and inhibits one or more activities of the f-circRNA. In an embodiment, the compound comprises an RNA sequence selected from the group consisting of antisense RNA, siRNA and shRNA. In a specific embodiment, the RNA sequence inhibits an f-circRNA activity. In another embodiment, the f-circRNA activity is selected from the group consisting of cellular proliferation, cellular transformation and carcinogenesis. In another embodiment, the RNA sequence mediates degradation of the f-circRNA. In another embodiment, the RNA sequence is chemically modified.

In another aspect, provided herein is a method of diagnosing a subject with a translocation-associated disorder or disease, comprising detecting in the subject the presence of a fusion-circular RNA (f-circRNA). In an embodiment, the disorder or disease is a cancer. In a specific embodiment, the cancer is selected from the group consisting of acute promyelocytic leukemia (APL), MLL-AF9-mediated leukemia, Ewing sarcoma and non-small cell lung carcinoma. In another embodiment, the f-circRNA is detected in a lysosomal fraction obtained from the subject.

In another aspect, provided herein is a method for treating a translocation-associated disorder or disease in a subject in need thereof, comprising contacting the subject with a therapeutic amount of an f-circRNA inhibiting agent effective to reduce one or more symptoms of the translocation-associated disorder or disease in the subject. In an embodiment, the f-circRNA inhibiting agent targets an f-circRNA back-splice junction of an f-circRNA. In another embodiment, the f-circRNA inhibiting agent is selected from the group consisting of antisense RNA, siRNA and shRNA. In another embodiment, the disorder or disease is a cancer. In a specific embodiment, the cancer is selected from the group consisting of APL, MLL-AF9-mediated leukemia, Ewing sarcoma and non-small cell lung carcinoma.

In another aspect, provided herein is a method of inducing apoptosis in a cancer cell, comprising contacting the cancer cell with a compound that inhibits an f-circRNA activity. In an embodiment, the compound binds to an f-circRNA back-splice junction of an f-circRNA. In an embodiment, the compound is exogenous RNA selected from the group consisting of antisense RNA, siRNA and shRNA.

In another aspect, provided herein is a method of treating cancer in a subject, comprising administering to the subject a compound that inhibits an f-circRNA activity. In an embodiment, the compound binds to an f-circRNA back-splice junction of an f-circRNA. In another embodiment, the compound is exogenous RNA selected from the group consisting of antisense RNA, siRNA and shRNA.

In another aspect, provided herein is a method of prognosing or diagnosing cancer in a subject, comprising detecting f-circRNA in an exosome of the subject.

In another aspect, provided herein is a method for increasing efficacy of a conventional anti-cancer therapy in a subject, comprising contacting a subject receiving conventional anti-cancer therapy with a compound that inhibits an f-circRNA activity in the subject. In an embodiment, the conventional anti-cancer therapy is one or both of chemotherapy and radiation. In another embodiment, the compound binds to an f-circRNA back-splice junction of an f-circRNA. In another embodiment, the compound is exogenous RNA selected from the group consisting of antisense RNA, siRNA and shRNA

In another aspect, provided herein is a method of reducing relapse from remission in a subject, comprising administering to the subject a compound that inhibits an f-circRNA activity in the subject. In an embodiment, the compound binds to an f-circRNA back-splice junction of an f-circRNA. In another embodiment, the compound is exogenous RNA selected from the group consisting of antisense RNA, siRNA and shRNA.

In another aspect, provided herein is a method of decreasing proliferation of a cell, comprising contacting the cell with a compound that inhibits an f-circRNA activity in the subject. In an embodiment, the compound binds to an f-circRNA back-splice junction of an f-circRNA. In another embodiment, the compound is exogenous RNA selected from the group consisting of antisense RNA, siRNA and shRNA.

In another aspect, provided herein is a method of decreasing cancer proliferation in a subject, comprising contacting the subject with a compound that inhibits an f-circRNA activity in the subject. In an embodiment, the compound binds to an f-circRNA back-splice junction of an f-circRNA. In another embodiment, the compound is exogenous RNA selected from the group consisting of antisense RNA, siRNA and shRNA.

In another aspect, provided herein is an isolated cell expressing an exogenous f-circRNA.

In another aspect, provided herein is a method of increasing proliferation rate in a cell, comprising contacting the cell with an exogenous f-circRNA.

In another aspect, provided herein is a method of transforming a cell, comprising contacting the cell with an exogenous f-circRNA.

In another aspect, provided herein is a cancer model, comprising a non-human organism expressing an exogenous f-circRNA.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIGS. 1A-G show that f-circRNAs occur in cancer cells as product of aberrant chromosomal translocations.

FIG. 1A depicts a schematic representation of the working hypothesis: f-circRNAs derive from chromosomal translocations. B.P. represents the breakpoint of the chromosomal translocation. As highlighted by the figure in the middle, f-circRNAs contained both the breakpoint of the translocation and the back-splice junction, which was unique for the circRNAs.

FIG. 1B depicts a schematic representation of the PML/RARα translocation that occurs in APL.

FIG. 1C depicts a PCR analysis for the identification of PML/RARα f-circRNA (f-circPR) from the RNA derived from bone marrow cells of leukemic patients harboring PML/RARα translocation, or from patients harboring different translocations used as controls. Divergent primers were used to detect f-circRNAs coming from the translocation. Convergent primers were used to detect the breakpoint of the translocation. Samples were treated with RNase R before PCR assay. Bands in squares were sequenced and ascertained as f-circRNAs.

FIG. 1D depicts a PCR analysis of the RNA derived from NB4 cells for the identification of f-circPR. RNA was treated or untreated with RNase R before PCR. THP1 cells were used as control.

FIG. 1E depicts a schematic representation of the MLL/AF9 translocation found in THP1 cell line.

FIG. 1F depicts a PCR analysis of RNA derived from THP1 cells, which were treated or untreated with RNase R. Divergent primers were used to detect f-circRNAs. Bands marked in squares were sequenced and ascertained as f-circRNAs. Sequences of found f-circM9 are highlighted in the schematic representation in the lower part of the panel. K562 cell line was used as control of the PCR assay.

FIG. 1G depicts a summary of the results of the analysis of f-circRNAs in the RNA-seq data.

FIGS. 2A-2I show that f-circRNAs are oncogenic RNAs that contribute to cellular transformation.

FIG. 2A depicts a schematic representation of the retroviral vectors used to express f-circRNAs.

FIG. 2B depicts a schematic representation of shRNAs used to target specifically the f-circRNAs (shCircRNAs) at their back-splice junction (BSJ).

FIG. 2C depicts proliferation curves of mouse embryonic fibroblasts (MEFs) transduced with empty vector (control) or with vector expressing either f-circM9 (upper panel) or f-circPR (lower panel). For each and every time point, the number of cells was detected upon staining with crystal violet, whose absorbance was then detected. (n=2 independent experiments, each performed in triplicate.)

FIG. 2D depicts a focus formation assay with MEF cells expressing empty vector as control, f-circM9 or f-circPR. Representative pictures are shown on the left, while the quantification of the transformed foci per well is shown on the right. (n=2 independent experiments, each performed in triplicate.)

FIG. 2E depicts PCR assays showing the levels of f-circPR and f-circM9 in MEsF upon the transduction with shSCR as control or a specific shRNAs that target the back-splice junction of the f-circRNAs (shCircRNAs). * indicates the specific band.

FIG. 2F depicts proliferation curves with MEFs that expressed either f-circM9 or f-circPR, that were concomitantly transduced with shSCR, shCircM9 or shCricPR. Representative pictures are shown in the upper panel, while the proliferation curve with transduced cells is shown in the graphs below. (n=2 independent experiments, each performed in triplicate.)

FIG. 2G depicts a schematic representation of the retroviral vector mutagenized at the splicing-donor site.

FIG. 2H depicts proliferation curves of MEFs transduced with the vector mutated at the splicing-donor site (control) or with the vector expressing either f-circM9 (right panel) or f-circPR (left panel). For each and every time point, the number of cells was detected upon staining with crystal violet, and its absorbance was detected. (n=3 independent experiments, each performed in triplicate.)

FIG. 2I depicts a focus formation assay with MEFs cells, which express either vectors mutated at the splicing donor site, or vectors expressing either f-circM9 or f-circPR. Representative pictures are shown in the upper panel, while the quantification of the number of transformed foci per well is shown in the lower panel. (n=3 independent experiments, each performed in triplicate).

FIGS. 3A-3G show that f-circM9 is oncogenic and contributes to leukemia progression.

FIG. 3A depicts a schematic representation of the generation of leukemic cells that express concomitantly MLL/AF9 cDNA, and f-circM9. In selected experiments, either the empty vector or the vector was mutagenized at the splicing-donor site was used as controls of the experiment.

FIG. 3B depicts the isolation and validation of leukemic cells (green fluorescent protein (GFP)+ and expressing MLL/AF9 cDNA) that were transduced with the empty vector or the vector containing f-circM9. Fluorescence activated cell sorting (FACS) was performed on cells using Discosoma sp. red fluorescent protein (dsRED).

FIG. 3C depicts the isolation and validation of leukemic cells (GFP+ and expressing MLL/AF9 cDNA) that were transduced with the vector containing f-circM9, or the vector mutagenized at the splicing-donor site (f-circM9-Mut). Cells were FACS-sorted for dsRED.

FIG. 3D depicts methyl-cellulose assays with leukemic cells (GFP+ and expressing MLL/AF9 cDNA) expressing either the empty vector (as control) or the vector containing f-circM9. Two rounds of re-plating were performed; 1000 cells were seeded in the plates at each round.

FIG. 3E depicts methyl-cellulose assays with leukemic cells (GFP+ and expressing MLL/AF9 cDNA) expressing f-circM9 or the vector mutagenized at the splicing-donor site (f-circM9-Mut, as control). Two rounds of re-plating were performed; 1000 cells were seeded in the plates at each round.

FIG. 3F depicts an analysis of expression of f-circM9 or the linear transcript (LinM9) in the leukemic cells derived from transplanted mice.

FIG. 3G depicts an analysis of the percentage of leukemic cells in the bone marrow of mice transplanted with leukemic cells expressing MLL/AF9 cDNA together with f-circM9 or MLL/AF9 cDNA together with the vector mutagenized at the splicing donor site (chart on the left). Sizes of spleens were derived from mice transplanted with leukemic cells expressing MLL/AF9 cDNA together with f-circM9 or MLL/AF9 cDNA together with the vector mutagenized at the splicing donor site. Representative pictures are shown in the middle panel, while the average size of the spleens is shown on the right.

FIGS. 4A-4G show that expression of f-circM9 in cancer cells confers resistance to treatments.

FIG. 4A depicts methyl-cellulose assays at the presence of arsenic trioxide (ATO), performed with leukemic cells (GFP+ and expressing MLL/AF9 cDNA) expressing either the empty vector (as control) or a vector containing f-circM9. Schematic representation of the assay is shown on the left. Relative number of colonies upon the treatment with ATO, and their morphology are shown in the middle panel, while the absolute number of counted colonies, both at steady state condition and under treatment with ATO is shown on the right.

FIG. 4B depicts an analysis of apoptosis in K562 cells upon transduction with an empty vector as control or a vector expressing f-circM9, upon treatment with Ara-C or ATO. Apoptotic/necrotic cells were analyzed as single or double positive for the incorporation of AnnexinV and 7-actinomycin D (7-AAD). Representative plots are shown on the left, while the quantification of the AnnexinV+ cells is shown in the chart on the right (n=4 independent experiments).

FIG. 4C depicts a schematic representation of the transplantation model to study the involvement of f-circM9 in conferring protection to cancer cells upon treatment with chemotherapy.

FIG. 4D depicts the expression of f-circM9 in leukemic cells extracted from the bone marrow (BM) of primary recipient mice, which were transplanted with leukemic cells expressing MLL/AF9 cDNA together with f-circM9 or together with the empty vector.

FIG. 4E depicts an analysis of the size of the spleens derived from secondary recipient mice transplanted with leukemic cells expressing MLL/AF9 cDNA together with f-circM9 or together with the empty vector, and subjected to treatment with AraC. Representative pictures are shown in the middle panel while the average spleen size is shown on the right.

FIG. 4F depicts an analysis of the percentage of leukemic cells in the spleen (left) and in the bone marrow (right) in secondary recipient mice transplanted with leukemic cells expressing MLL/AF9 cDNA together with f-circM9 or MLL/AF9 cDNA together with the empty vector, and subjected to treatment with AraC.

FIG. 4G depicts an analysis of necrotic/apoptotic leukemic cells in mice transplanted with leukemic cells expressing MLL/AF9 cDNA together with f-circM9 or MLL/AF9 cDNA together with the empty vector, and subjected to treatment with AraC. The histograms represent the GFP+ leukemic cells; cells positive for the expression of AnnexinV and 7AAD were analyzed within the GFP+ population. Plots on the left are representative of the results, while the quantification is shown on the right.

FIGS. 5A-5G depict an analysis of the endogenous f-circM9 and its role in the maintenance of cancer cells.

FIG. 5A depicts the fractionation nuclei/cytoplasms of THP1, and analysis of LinM9 and f-circM9 in the two distinct fractions. Laminin and beta-actin were used to control the efficiency of the fractionation assay.

FIG. 5B depicts a quantification of LinM9 and f-circM9 in total RNA or RNA extracted from the nucleus or the cytoplasm of THP1 cells. Values are expressed as number of molecules per ng of RNA extracted.

FIG. 5C depicts an analysis of the stability of LinM9 (left) and f-circM9 (right) in THP1 cells treated with actinomyosin D.

FIG. 5D depicts a schematic representation of the shRNAs used to specifically target f-circM9 (shCircM9_1_4) at the back-splice junction (BSJ) (upper panel), and validation by PCR of the capacity of shCircM9 to knock down the f-circM9. Convergent primers were used to detect the linear transcript (LinM9), while divergent primers were used to detect the f-circM9.

FIG. 5E depicts a Western blot analysis of endogenous MLL and AF9 proteins in THP1 cells upon transduction with shCircM9 vectors. Representative pictures are shown on the upper panel, while the quantification of the bands is shown on the lower panel.

FIG. 5F depicts a schematic representation (upper panel) of the primers used to identify MLL and AF9 transcripts. Primers were designed in a portion of the transcript that is not included in the f-circM9. Lower charts show the expression of MLL and AF9 linear transcripts.

FIG. 5G depicts an analysis of the apoptosis in THP1 cells upon transduction with an shSCR as control and specific shCircRNAs against f-circM9 or f-circPR. Representative plots are shown on the left, while the quantification of the AnnexinV+ cells is shown in the chart on the right.

FIGS. 6A-6B depict a schematic representation of the role of f-circRNAs.

FIG. 6A depicts a schematic representation of the origin of f-circRNAs from chromosomal translocations and their co-existence with oncogenic fusion-proteins in tumor cells.

FIG. 6B depicts a schematic representation of the role of f-circRNAs in tumor progression and resistance to therapy.

FIGS. 7A-7E depict characterization of f-circRNAs derived from PML/RARα and MLL/AF9 chromosomal translocations.

FIG. 7A depicts a schematic representation of the PML/RARα translocation that occurs in APL as well as the primers (both convergent and divergent) used to detect linear mRNA of PML/RARα and f-circPR.

FIG. 7B depicts a schematic representation and results of sequencing of PCR-amplified products obtained using divergent primers on PML/RARα. SEQ ID NO:1 is set forth as the top nucleotide sequence, SEQ ID NO:2 is set forth as the bottom nucleotide sequence.

FIG. 7C depicts an analysis of f-circPR in an NB4 cell line. Highlighted PCR products were extracted and analyzed by Sanger sequencing.

FIG. 7D depicts a schematic representation of the MLL/AF9 translocation as well as the of primers (both convergent and divergent) used to detect linear mRNA of MLL/AF9 and f-circM9.

FIG. 7E depicts a schematic representation and results of sequencing of PCR-amplified products obtained using divergent primers on MLL/AF9. SEQ ID NO:3 is set forth as the bottom nucleotide sequence, SEQ ID NO:4 is set forth as the top nucleotide sequence.

FIGS. 8A-8F depict F-circRNAs derived from EWS/FLI1 and EML4/ALK1 chromosomal translocations as well as bioinformatics analysis of f-circRNAs.

FIG. 8A depicts a schematic representation of the EWS/FLI1 translocation as well as the primers (both convergent and divergent) used to detect linear mRNA of EWS/FLI1 and f-circEF1 (on the right). The amplification of linear transcript EWS/FLI1 was performed with convergent primers. The amplification of f-circEF1 was performed with divergent primers. The left part of the picture shows PCR-amplified products obtained using convergent primers (f-LinEF1) or divergent primers (f-circEF1) on EWS/FLI1.

FIG. 8B depicts a schematic representation and results of sequencing of PCR-amplified products obtained using divergent primers on EWS/FLI1. The nucleotide sequence is set forth as SEQ ID NO:5.

FIG. 8C depicts a schematic representation of the EML4/ALK1 translocation as well as the primers (both convergent and divergent) used to detect linear mRNA of EML4/ALK1 or f-circEA1. The amplification of linear transcript EML4/ALK1 (f-linEA1) was performed with convergent primers. The amplification of f-circEA1 was performed with divergent primers. The nucleotide sequence is set forth as SEQ ID NO:6.

FIG. 8D depicts a schematic representation and results of sequencing of PCR-amplified products obtained using convergent primers (f-LinEA1) or divergent primers (f-circEA1) on EML4/ALK1.

FIG. 8E depicts a schematic representation of the bioinformatics pipelines used for the analysis of f-circRNAs.

FIG. 8F depicts visualization of f-circRNA reads from RNA-Seq data from a THP1 cell line. The top of the figure indicates the bowtie mapping parameters (n is the number of allowed mismatches within a length 1; w is the minimum number of reads required from each fusion partner (anchor length)). The AF9 sequence of the f-circRNAs is displayed on the left side of the back-splicing junction, with the MLL sequence shown on the right. By adjusting the 1 parameter, the consensus and specificity of the mappings were increased (bottom portion). Figure discloses “AAGCAAGCTAAAGCTGTGAAAAAGAAAGAGAAAAAGTCT” as SEQ ID NO: 51.

FIGS. 9A-9F depict expression of f-circRNAs in MEFs.

FIG. 9A depicts a schematic representation of exons and Alu-sequences inserted into the retroviral vectors which were used to express f-circRNAs (f-circPR and f-circM9).

FIG. 9B depicts up-regulation, measured by RT-qPCR, of the expression levels of f-circPR (chart on the left) and of f-circM9 (chart on the right) in MEFs cells upon transduction with expressing retroviral vectors.

FIG. 9C depicts up-regulation, measured by PCR, of the expression levels of f-circPR (on the left) and of f-circM9 (on the right) in MEFs cells upon transduction with expressing retroviral vectors.

FIG. 9D depicts expression levels of f-circPR (on the left) and of f-circM9 (on the right) in MEFs cells upon transduction with retroviral vector expressing f-circRNAs or the spicing-donor mutant retroviral vector (f-circRNA-mut).

FIG. 9E depicts western blot analysis and relative quantifications (lower charts) of the activation of Akt (left) and Erk1/2 (right) in MEFs cells upon transduction with retroviral vector expressing f-circPR or the spicing-donor mutant retroviral vector (f-circPR-mut).

FIG. 9F depicts western blot analysis and relative quantifications (lower charts) of the activation of Akt (left) and Erk1/2 (right) in MEFs cells upon transduction with retroviral vector expressing f-circM9 or the spicing-donor mutant retroviral vector (f-circM9-mut).

FIGS. 10A-10B depict expression of the f-circM9 in primary leukemic cells.

FIG. 10A depicts a schematic representation of the generation of leukemic cells that express MLL/AF9 cDNA. These cells can be isolated because of expression of GFP.

FIG. 10B depicts analysis of the percentage of leukemic cells in the bone marrow of mice transplanted with leukemic cells expressing MLL/AF9 cDNA together with f-circM9 or MLL/AF9 cDNA together with the vector mutagenized at the splicing donor site (chart on the left). The pictures show the spleens derived from mice transplanted with leukemic cells expressing MLL/AF9 cDNA together with f-circM9 or MLL/AF9 cDNA together with the vector mutagenized at the splicing donor site (f-circM9-mut). Representative pictures are shown in the middle panel while the average size of the spleens is shown on the right.

FIG. 11 shows that expression of f-circM9 confers protection to leukemic cells under treatment, showing total number of cells growing in colonies in the methyl-cellulose plates. Leukemic cells expressing the cDNA of MLL/AF9 were transduced with empty vector or a vector expressing f-circM9. Cells were then plated in methylcellulose, and untreated (N.T.) or treated with ATO.

FIG. 12 depicts the lentiviral backbone vector pLKO.1.

FIGS. 13A-13F show that knock down of f-circRNAs drive cells to apoptosis.

FIG. 13A depicts PCR assays showing the levels of endogenous f-circPR (using divergent primers), and f-LinPR (using convergent primers) in NB4 cells upon their transduction with an shSCR as control and specific shCircRNAs against the f-circPR (shCircPR1-3). * indicates the specific band.

FIG. 13B depicts western blot analysis (left) of the expression of the protein PML, RARα and the fusion protein PML/RARα (indicated with the arrow) in NB4 cells, upon transduction with an shSCR as control, and specific shCircRNAs against the f-circPML/RARα (shCircPR1-3), and in kasumi-1 cell lines used as control. Charts on the right show the quantification of RARα and PML/RARα proteins detected by western blot.

FIG. 13C depicts PCR assays showing the relative expression of PML/RARα mRNA in NB4 cells upon their transduction with an shSCR as control and specific shCircRNAs against the f-circPR (shCircPR1-3). To detect PML/RARα mRNA, primers were located in PML and RARα exons that do not take part to the f-circPR.

FIG. 13D depicts PCR assays showing the levels of endogenous f-circPR (using divergent primers) and f-LinPR (using convergent primers) in NB4 cells upon transduction with an shSCR as control, the specific shCircRNAs against the f-circPR (shCircPR1-3), and the shRNA against f-circM9 (shCircM9-2).

FIG. 13E depicts analysis of apoptosis in NB4, U937, HL60 and Kasumi cells upon transduction with an shSCR as control, the specific shRNA against the f-circPR (shCircPR1), and the specific shRNA against f-circM9 (shCircM9-2). Apoptotic/necrotic cells were analyzed as double positive for the incorporation of AnnexinV and 7AAD. Representative plots are shown on the left, while the quantification of the AnnexinV+ cells is shown in the charts on the right (n=3 independent experiments). Left chart represents AnnexinV+ cells in NB4 cell line, right chart represent AnnexinV+ cells in HL60 cell line.

FIG. 13F depicts detection of the mRNA levels of p21 and p27 in NB4 cells transduced with shSCR or shCircRNA against PML/RARα.

FIG. 14 depicts the assembly strategy for sub-cloning the expression vector of circPML/RARα according to certain embodiments. (NEBuilder, website: nebuilder.neb.com.) Figure discloses SEQ ID NOS 45-50, respectively, in order of appearance.

FIG. 15 depicts the assembly strategy for sub-cloning the vector for the expression of circMLL/AF9 according to certain embodiments. Figure discloses SEQ ID NOS 37-44, respectively, in order of appearance.

### DETAILED DESCRIPTION OF CERTAIN EXEMPLARY EMBODIMENTS

Novel f-circRNAs and complements thereof are provided. Diagnostic and therapeutic methods using f-circRNAs (and complements thereof) of the invention are provided. Reagents that target and downregulate (e.g., an f-circRNA inhibiting agent) or upregulate f-circRNAs are also provided.

Generally, nomenclature used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the invention may be more readily understood, certain terms are first defined.

As used herein, “circular RNAs” or “circRNAs” refer to a type of RNA which forms a covalently closed continuous loop via joining of the 3′ and 5′ ends of two exons or exon fragments. CircRNAs typically arise from otherwise protein-coding genes, but circRNAs have not been shown to be translated in vivo. Accordingly, circRNAs are considered to be noncoding RNA. Because circular RNAs do not have 5′ or 3′ ends, they are generally resistant to exonuclease-mediated degradation.

The biogenesis of circRNAs is actively regulated and favored by the presence of specific and repetitive sequences and ribosomal binding protein (RBP) binding sites within the introns upstream and downstream of the circularizing exons (Jeck et al., 2013; Ashwal-Fluss et al., 2014; Barrett et al., 2015; Conn et al., 2015; Liang and Wilusz, 2014; Zhang et al., 2014). Thus, circRNAs are the result of a pro-active back-splice event, in which the 3′-tail of one exon is joined to the 5′-head of an up-stream exon (Jeck et al., 2013).

The present invention is based in part on the discovery of novel, aberrant non-coding RNAs created by the fusion of two or more translocated genes: fusion-circRNAs (f-circRNAs). As used herein, an “f-circRNA” refers to a circular RNA that includes one or more exons or exon fragments from at least two distinct genes which are generated as a consequence of one or more chromosomal translocations. An f-circRNA may contain one, two, three, four, five or more exons or exon fragments from one or more genes. An f-circRNA, e.g., an f-circRNA associated with a particular translocation disorder, may be caused by a variety of combinations of exons corresponding to the translocated genes.

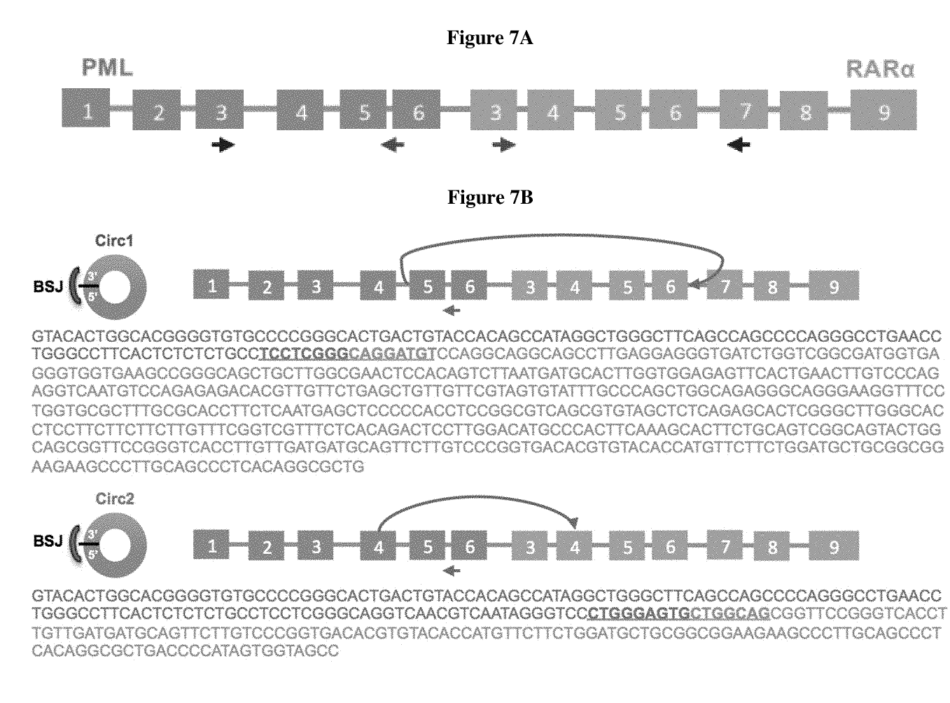

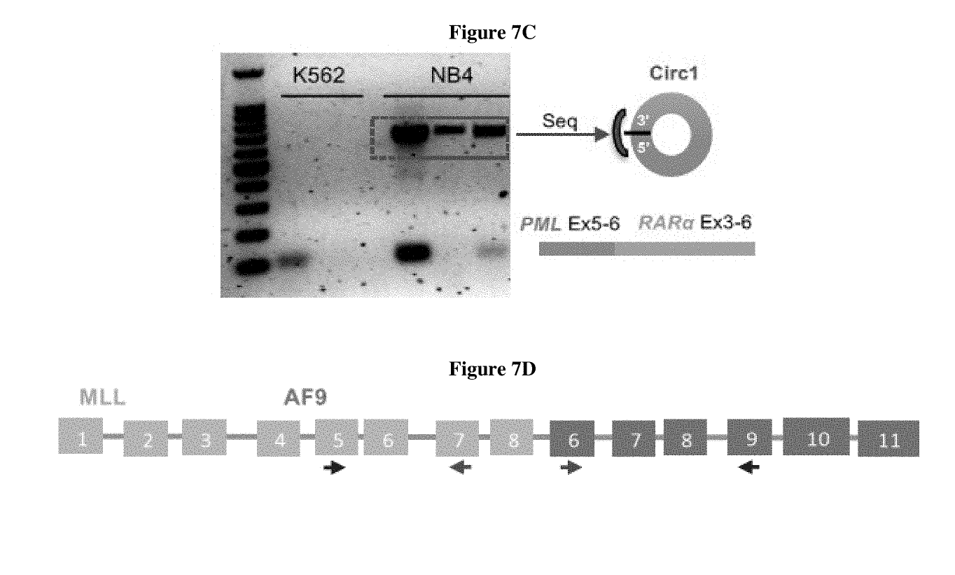

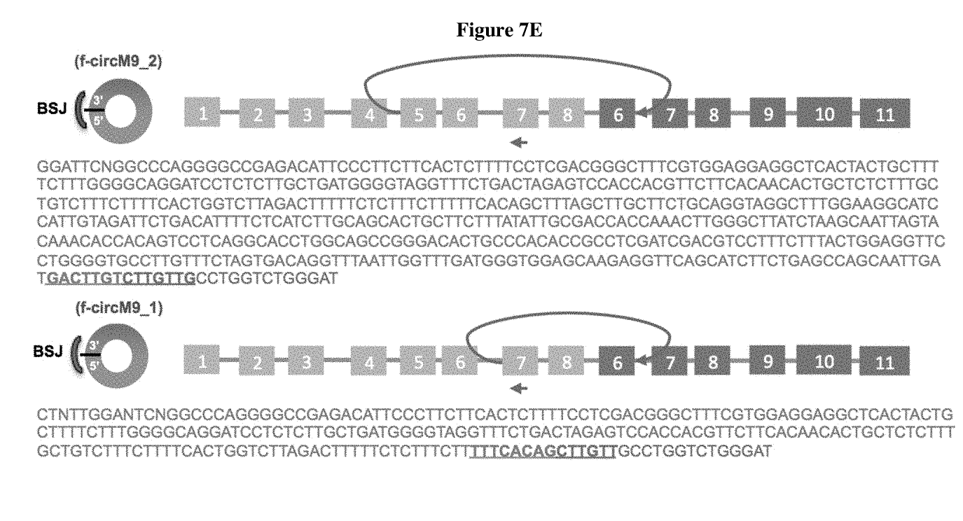

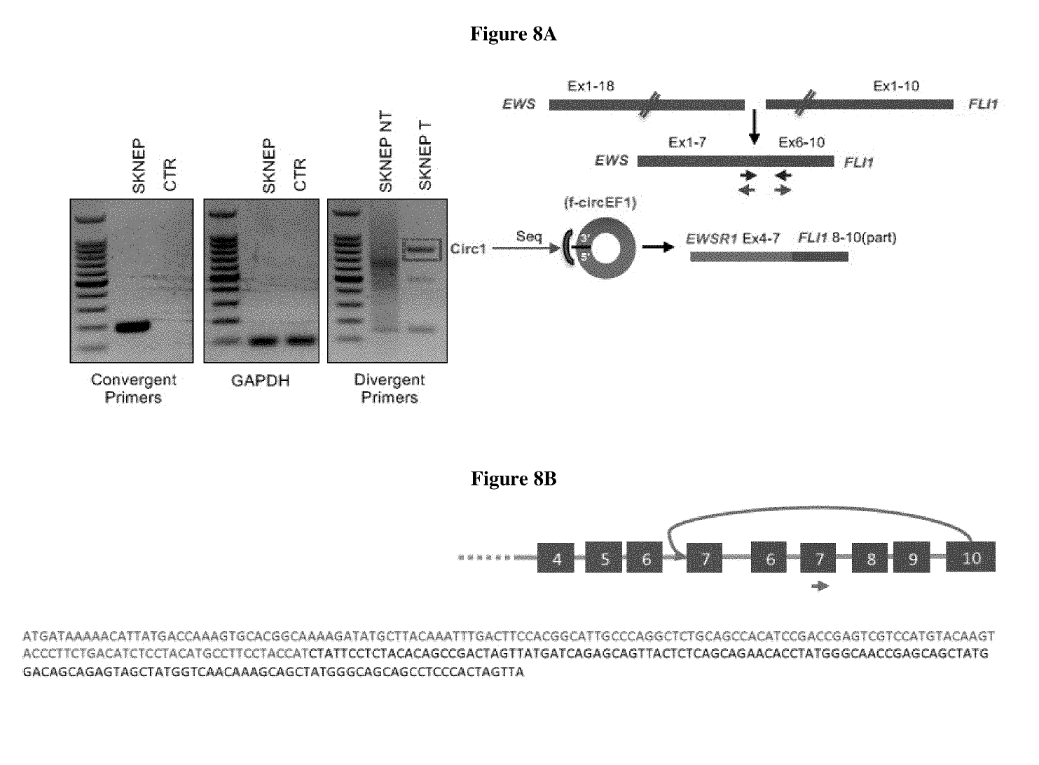

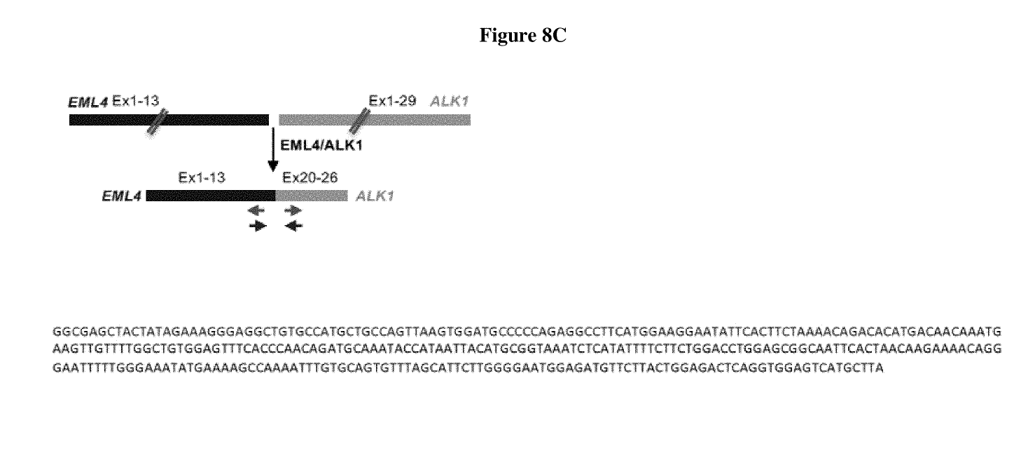

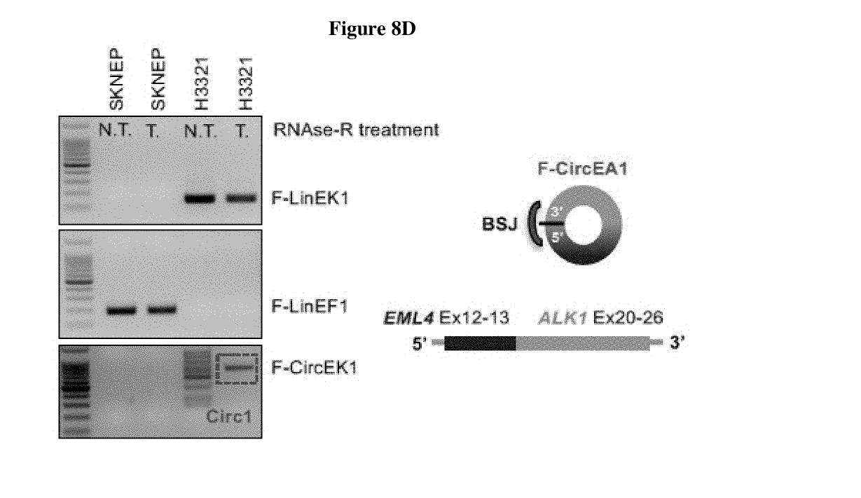

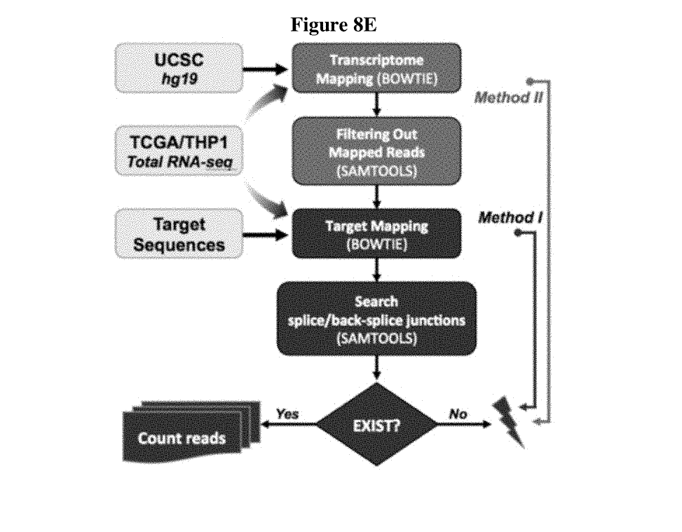

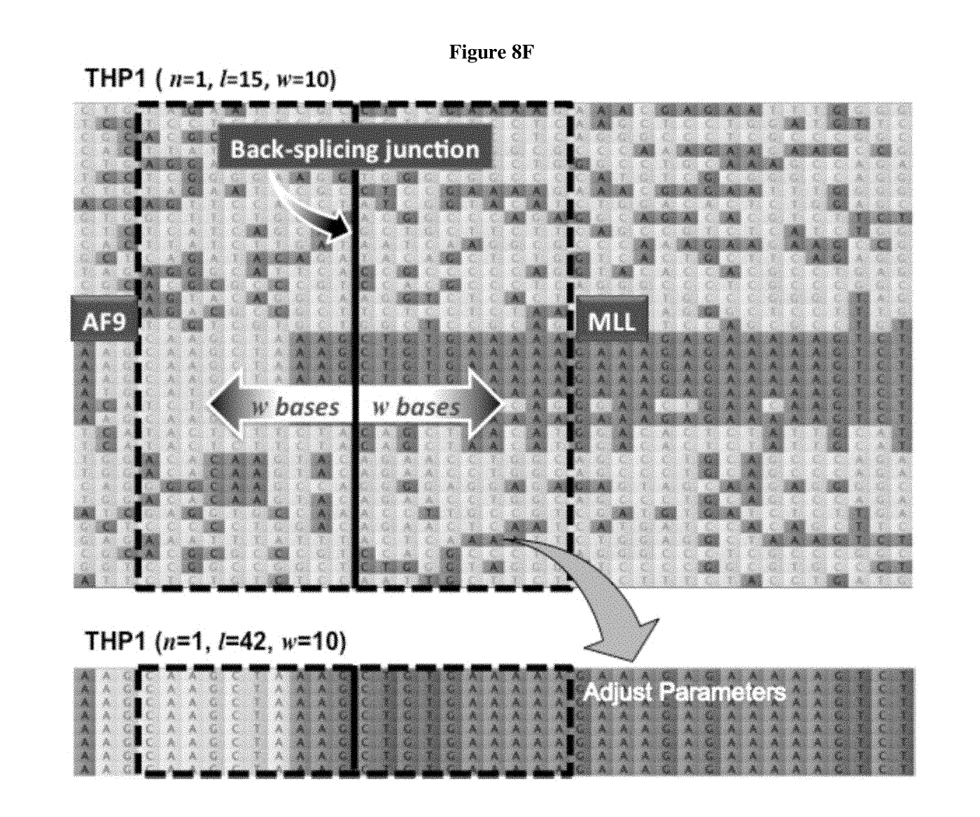

The f-circRNAs of the invention further include an f-circRNA back-splice junction that joins two exons or portions of two exons corresponding to two or more different genes. As used herein, the term “f-circRNA back-splice junction” refers to a region formed between the 5′-head of a first exon and the 3′-tail of a second exon in an f-circRNA. An f-circRNA back-splice junction represents a unique sequence generated by splicing, which sequence should not be present in the genome, mRNAs or circRNAs of an organism. As such, an f-circRNA back-splice junction serves as a highly specific region of the f-circRNA for targeting with an f-circRNA inhibiting agent of the invention. Exemplary f-circRNAs including back-splice junctions are described in FIGS. 7A-7E and 8A-8D.

In certain exemplary embodiments, an f-circRNA of the invention comprises PML and RARα exons with a back-splice junction between the 5′ head of PML exon 5 and the 3′ tail of RARα exon 6, e.g., SEQ ID NO:1 (See FIG. 7B), or an f-circRNA having at least 80% homology, e.g., 80%, 81%, 82%, 83%, 84%, 85%, 86%, 87%, 88%, 89%, 90%, 91%, 92%, 93%, 94%, 95%, 96%, 97%, 98% or 99% homology to SEQ ID NO:1. In certain exemplary embodiments, an f-circRNA of the invention comprises PML and RARα exons with a back-splice junction between the 5′ head of PML exon 4 and the 3′ tail of RARα exon 4, e.g., SEQ ID NO:2 (See FIG. 7B), or an f-circRNA having at least 80% homology, e.g., 80%, 81%, 82%, 83%, 84%, 85%, 86%, 87%, 88%, 89%, 90%, 91%, 92%, 93%, 94%, 95%, 96%, 97%, 98% or 99% homology to SEQ ID NO:2.

In certain exemplary embodiments, an f-circRNA of the invention comprises MLL and AF9 exons with a back-splice junction between the 5′ head of MLL exon 7 and the 3′ tail of AF9 exon 6, e.g., SEQ ID NO:3 (See FIG. 7E), or an f-circRNA having at least 80% homology, e.g., 80%, 81%, 82%, 83%, 84%, 85%, 86%, 87%, 88%, 89%, 90%, 91%, 92%, 93%, 94%, 95%, 96%, 97%, 98% or 99% homology to SEQ ID NO:3. In certain exemplary embodiments, an f-circRNA of the invention comprises MLL and AF9 exons with a back-splice junction between the 5′ head of MLL exon 5 and the 3′ tail of AF9 exon 6, e.g., SEQ ID NO:4 (See FIG. 7E), or an f-circRNA having at least 80% homology, e.g., 80%, 81%, 82%, 83%, 84%, 85%, 86%, 87%, 88%, 89%, 90%, 91%, 92%, 93%, 94%, 95%, 96%, 97%, 98% or 99% homology to SEQ ID NO:4.

In certain exemplary embodiments, an f-circRNA of the invention comprises EWSR1 and FLI1 exons with a back-splice junction between the 5′ head of EWSR1 exon 7 and the 3′ tail of FLI1 exon 10, e.g., SEQ ID NO:5 (See FIG. 8B), or an f-circRNA having at least 80% homology, e.g., 80%, 81%, 82%, 83%, 84%, 85%, 86%, 87%, 88%, 89%, 90%, 91%, 92%, 93%, 94%, 95%, 96%, 97%, 98% or 99% homology to SEQ ID NO:5.

In certain exemplary embodiments, an f-circRNA of the invention comprises EML4 and ALK1 exons with a back-splice junction between the 5′ head of EML4 exon 12 and the 3′ tail of ALK1 exon 26, e.g., SEQ ID NO:6 (See FIG. 8C), or an f-circRNA having at least 80% homology, e.g., 80%, 81%, 82%, 83%, 84%, 85%, 86%, 87%, 88%, 89%, 90%, 91%, 92%, 93%, 94%, 95%, 96%, 97%, 98% or 99% homology to SEQ ID NO:6.

F-circRNA back-splice junctions can range in size from about 5 to about 25 nucleic acids in length, from about 10 to about 20 nucleic acids in length, from about 12 to about 18 nucleic acids in length, or from about 14 to about 16 nucleic acids in length. In certain exemplary embodiments, an f-circRNA back-splice junction is about 5, about 6, about 7, about 8, about 9, about 10, about 11, about 12, about 13, about 14, about 15, about 16, about 17, about 18, about 19, about 20, about 21, about 22, about 23, about 24 or about 25 nucleic acids in length. In certain exemplary embodiments, an f-circRNA back-splice junction of the invention comprises any of SEQ ID NOs:7-10 (SEQ ID NO:7, 5′-TCCTCGGGCAGGATGT-3′; SEQ ID NO:8, 5′-CTGGGAGTGCTGGCAG-3′; SEQ ID NO:9, 5′-GACTTGTCTTGTTG-3′; SEQ ID NO:10, 5′-TTTCACAGCTTGTT-3′) or a sequence having at least 80% homology, e.g., 80%, 81%, 82%, 83%, 84%, 85%, 86%, 87%, 88%, 89%, 90%, 91%, 92%, 93%, 94%, 95%, 96%, 97%, 98% or 99% homology to any of SEQ ID NOs:7-10.

F-circRNAs of the invention can mediate one or more translocation-associated disorders (e.g., cancer-associated translocation disorders and/or non-cancerous translocation disorders); tumorigenesis; carcinogenesis; cellular proliferation; cellular transformation; cellular immortalization; maintenance of cancer cells; resistance to cancer therapy; and/or regulation of apoptosis.

As used herein, a “chromosomal translocation” refers to a chromosome alteration in which a whole chromosome or segment of a chromosome becomes attached to or interchanged with another whole chromosome or segment, the resulting hybrid segregating together at meiosis. Chromosomal translocation include, but are not limited to, balanced translocations, unbalanced translocations, Robertsonian translocations and insertional translocations.

Balanced translocations as a whole are thought to occur at a rate of about 1 in 500 in the general population. Balanced translocations happen when breaks occur in two or more different chromosomes and the resulting fragments of DNA swap places. No chromosome material has been lost or gained and so the vast majority of carriers of a balanced reciprocal translocation do not have any symptoms. Balanced translocations are usually not associated with phenotypic abnormalities, although gene disruptions at the breakpoints of the translocation can, in some cases, cause adverse effects, including some known genetic disorders.

Unbalanced translocations, in which there is loss or gain of chromosome material, nearly always yield an abnormal phenotype.

Robertsonian translocations occur when the short arm of certain chromosomes (e.g., chromosomes 13, 14, 15, 21 or 22) are lost and the remaining long arms fuse together. Loss of the short arms of these chromosomes typically does not cause any symptoms. A person with a Robertsonian translocation has a total chromosome number of 45. Robertsonian translocations are relatively common in the general population (about 1 in 1000), the most frequent being fusion of the long arms of chromosomes 13 and 14. The significance of a Robertsonian Translocation is the risk of miscarriage or of producing children with an unbalanced chromosome make-up.

Insertions occur when a segment of one chromosome is inserted into a gap in another chromosome. Balanced insertional rearrangement typically do not produce a noticeable phenotype in a subject (unless a critical gene is disrupted at the breakpoints) but the subject is at risk of producing offspring with either a deletion or a duplication of chromosome material.

Chromosomal translocations are considered as the primary cause for many cancers including lymphoma, leukemia and some solid tumors. Chromosomal translocations in certain cases can result either in the fusion of genes or in bringing genes close to enhancer or promoter elements, hence leading to their altered expression. Moreover, chromosomal translocations can be used as diagnostic markers for cancer and its therapeutics.

In certain embodiments, f-circRNAs are associated with cancer-associated translocation disorders. Accordingly, certain embodiments of the invention are directed to the diagnosis and/or treatment of one or more disorders associated with aberrant cellular proliferation, e.g., cancer. By “treatment of aberrant cellular proliferation” is meant use of an inhibitor of an f-circRNA (e.g., an siRNA) of the invention in a pharmaceutical composition to inhibit aberrant cellular proliferation. As used herein, the term “disorder associated with aberrant cellular proliferation” includes, but is not limited to, disorders characterized by undesirable or inappropriate proliferation of one or more subset(s) of cells in a multicellular organism (e.g., cancer).

The term “cancer” refers to various types of malignant neoplasms, most of which can invade surrounding tissues, and may metastasize to different sites (PDR Medical Dictionary 1st edition (1995)). The terms “neoplasm” and “tumor” refer to an abnormal tissue that grows by cellular proliferation more rapidly than normal and continues to grow after the stimuli that initiated proliferation is removed (PDR Medical Dictionary 1st edition (1995)). Such abnormal tissue shows partial or complete lack of structural organization and functional coordination with the normal tissue which may be either benign (i.e., benign tumor) or malignant (i.e., malignant tumor).

Cancers are classified by the type of cell that the tumor cells resemble, and is therefore presumed to be the origin of the tumor. These types include carcinomas, sarcomas, lymphomas and leukemias, germ cell tumors and blastomas. As used herein, a “carcinoma” refers to a cancer derived from epithelial cells. Carcinomas include many of the most common cancers, particularly in the aged, and include nearly all those developing in the breast, prostate, lung, pancreas, and colon. As used herein, a “sarcoma” refers to a cancer arising from connective tissue (e.g., bone, cartilage, fat, nerve), which develops from cells originating in mesenchymal cells outside the bone marrow. As used herein, “lymphoma” and “leukemia” refer to two classes of cancer that arise from hematopoietic cells in the marrow that typically to mature in the lymph nodes and blood, respectively. Leukemia is the most common type of cancer in children accounting for about 30%. As used herein a “germ cell tumor” refers to a cancer derived from pluripotent cells, most often presenting in the testicle or the ovary (seminoma and dysgerminoma, respectively). As used herein, a “blastomas” refers to a cancer derived from immature precursor cells or embryonic tissue. Blastomas are more common in children than in older adults.

In certain embodiments, cancers of the invention are those that are associated with one or more chromosomal translocations. Cancers associated with chromosomal translocations include, but are not limited to Burkitt's lymphoma (t(8;14)(q24;q32)), mantle cell lymphoma (t(11;14)(q13;q32)), follicular lymphoma (t(14;18)(q32;q21)), papillary thyroid cancer (t(10;(various))(q11;(various))), follicular thyroid cancer (t(2;3)(q13;p25)), acute myeloblastic leukemia with maturation (t(8;21)(q22;q22)), chronic myelogenous leukemia (CML) (t(9;22)(q34;q11); t(9;12)(p24;p13); Philadelphia chromosome), acute lymphoblastic leukemia (ALL) (t(9;22)(q34;q11); t(9;12)(p24;p13); t(12;21)(p12;q22); t(17;19)(q22;p13); Philadelphia chromosome), acute promyelocytic leukemia (t(15;17)(q22;q21)), acute myeloid leukemia (t(12;15)(p13;q25)), congenital fibrosarcoma (t(12;15)(p13;q25)), secretory breast carcinoma (t(12;15)(p13;q25)), mammary analogue secretory carcinoma of salivary glands (t(12;15)(p13;q25)), mucosa-associated lymphoid tissue lymphoma (MALT lymphoma) (t(11;18)(q21;q21)), dermatofibrosarcoma protuberans (DFSP) (t(17;22)), analplastic large cell lymphoma (t(2;5)(p23;q35)), Ewing's sarcoma (t(11;22)(q24;q11.2-12)), acute myelogenous leukemia (t(1;12)(q21;p13)), synovial sarcoma (t(X;18)(p11.2;q11.2)), oligodendroglioma (t(1;19)(q10;p10)), oligoastrocytoma (n1;19)(q10;p10)), low-grade fibromyxoid sarcoma (t(7,16) (q32-34;p11); t(11,16) (p11;p11)), dermatofibrosarcoma (t(17;22)) and the like. In preferred embodiments, f-circRNAs are associated with one or more of acute promyelocytic leukemia (APL) (PML/RARα translocation), MLL-AF9-mediated leukemia (MLL/AF9 translocation), Ewing sarcoma (EWSR1-FL11 translocation), non-small cell lung carcinoma (AML4/ALK1 translocation) and the like.

In certain embodiments, f-circRNAs are associated with non-cancer-associated translocation disorders. Accordingly, certain embodiments of the invention are directed to the treatment of one or more non-cancer disorders associated with chromosomal translocations including, but not limited to, infertility, XX male syndrome, translocation Down syndrome, schizophrenia (t(1;11)(q42.1;q14.3)) and the like.

By “therapeutic amount” is meant an amount that, when administered to a patient suffering from cancer or a non-cancer disorder associated with chromosomal translocation, is sufficient to cause a qualitative or quantitative reduction in the symptoms of a cancer or a non-cancer disorder associated with chromosomal translocation as described herein.

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2,2N,N-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively), linear or circular (e.g., circRNA, f-circRNA). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference. Preferably, a siRNA comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

As used herein, the term “short hairpin RNA” (“shRNA”) (also known as “small hairpin RNAs”) refers to an RNA (or RNA analog) including a first portion and a second portion, having sufficient complementarity to anneal or hybridize to form a duplex or double-stranded stem portion. The two portions need not be fully or perfectly complementary. The first and second “stem” portions are connected by a portion having a sequence that has insufficient sequence complementarity to anneal or hybridize to other portions of the shRNA. This latter portion is referred to as a “loop” portion in the shRNA molecule. shRNA molecules are processed to generate siRNAs. shRNAs can also include one or more bulges, i.e., extra nucleotides that create a small nucleotide “loop” in a portion of the stem, for example a one-, two- or three-nucleotide loop. The stem portions can be the same length, or one portion can include an overhang of, for example, 1-5 nucleotides. The overhanging nucleotides can include, for example, uracils (Us), e.g., all Us. Such Us are notably encoded by thymidines (Ts) in the shRNA-encoding DNA which signal the termination of transcription.

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; 0- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

The term “oligonucleotide” refers to a short polymer of nucleotides and/or nucleotide analogs. The term “RNA analog” refers to an polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Preferred RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

An RNAi agent, e.g., an RNA silencing agent (e.g., an f-circRNA inhibiting agent), having a strand which is “sufficiently complementary to a target f-circRNA sequence to direct target-specific RNA interference (RNAi)” means that the strand has a sequence sufficient to trigger the destruction of the target f-circRNA inhibiting agent by the RNAi machinery or process.

As used herein, the term “isolated RNA” (e.g., “isolated siRNA,” “isolated siRNA precursor,” “isolated f-circRNA” or “isolated complement of f-circRNA”) refers to RNA molecules which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

The term “discriminatory RNA silencing” refers to the ability of an RNA molecule to substantially inhibit the expression of a “first” or “target” polynucleotide sequence while not substantially inhibiting the expression of a “second” or “non-target polynucleotide sequence,” e.g., when both polynucleotide sequences are present in the same cell. In certain embodiments, the target polynucleotide sequence corresponds to a target f-circRNA (e.g., a back-splice junction), while the non-target polynucleotide sequence corresponds to a non-target gene, e.g., mRNA, circRNA or the like.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the term “transgene” refers to any nucleic acid molecule, which is inserted by artifice into a cell, and becomes part of the genome of the organism that develops from the cell. Such a transgene may include a gene that is partly or entirely heterologous (i.e., foreign) to the transgenic organism, or may represent a gene homologous to an endogenous gene of the organism. The term “transgene” also means a nucleic acid molecule that includes one or more selected nucleic acid sequences, e.g., DNAs, that encode one or more engineered RNA precursors, to be expressed in a transgenic organism, e.g., animal, which is partly or entirely heterologous, i.e., foreign, to the transgenic animal, or homologous to an endogenous gene of the transgenic animal, but which is designed to be inserted into the animal's genome at a location which differs from that of the natural gene. A transgene includes one or more promoters and any other DNA, such as introns, necessary for expression of the selected nucleic acid sequence, all operably linked to the selected sequence, and may include an enhancer sequence. In preferred embodiments, a transgene encodes an f-circRNA or a complement thereof.

As used herein, the term “target f-circRNA” refers to an f-circRNA whose expression, in certain embodiments of the invention, is to be substantially inhibited or “silenced.” This silencing can be achieved by cleaving the f-circRNA or by otherwise inhibiting the f-circRNA. The term “non-target RNA” is an RNA whose expression is not to be substantially inhibited or silenced, e.g., mRNA, circRNA and the like. In exemplary embodiments, the f-circRNA is inhibited or silenced by an f-circRNA inhibiting agent.

As used herein, an “f-circRNA inhibiting agent” refers to any agent that cleaves an f-circRNA, prevents formation of an f-circRNA and/or inhibits one or more activities of an f-circRNA. In certain embodiments, an f-circRNA inhibiting agent directly interacts with an f-circRNA and cleaves or otherwise renders inactive the f-circRNA. Preferred examples of agents that cleave f-circRNAs are RNAi agents, e.g., siRNAs and/or shRNAs. In other embodiments, an f-circRNA inhibiting agent interacts with a member of the f-circRNA pathway to specifically inhibit splicing in the f-circRNA. A preferred example of an agent that inhibits splicing in the f-circRNA pathway is an agent that sterically inhibits binding to the DNA in a region necessary for the formation of an f-circRNA, e.g., at one or more regions of the DNA encoding one or introns that give rise to f-circRNAs (e.g., at one or more Alu elements). In other embodiments, an f-circRNA inhibiting agent interacts with one or more downstream factors involved the f-circRNA pathway.

As used herein, the term “sample population” refers to a population of individuals comprising a statistically significant number of individuals. For example, the sample population may comprise 50, 75, 100, 200, 500, 1000 or more individuals. In particular embodiments, the sample population may comprise individuals which share at least on common disease phenotype or chromosomal translocation.

The phrase “examining the function of f-circRNA in a cell or organism” refers to examining or studying the expression, activity, function or phenotype arising therefrom.

As used herein an “animal model” refers to any non-human animals and biological samples derived therefrom that can be used to study a translocation-associated disease and/or disorder, e.g., cancer. Animal models include non-human animals that express a translocation-associated cancer (e.g., animal models of cancer). Animal models also include non-human animals that express an exogenous f-circRNA. In certain embodiments, an animal that expresses an exogenous f-circRNA exhibits one or more symptom of a translocation-associated disease and/or disorder, e.g., tumorigenesis, carcinogenesis, cellular proliferation, cellular transformation, cellular immortalization, resistance to cancer therapy, and/or dysregulation of apoptosis. Animal models also include non-human animals that express an exogenous complement of an f-circRNA. Animal models also include non-human animals that express an exogenous f-circRNA and an exogenous complement of an f-circRNA. Animal models are useful for screening modulators, e.g., antagonists and/or agonists of one or more f-circRNA activities. Exemplary animal models include, but are not limited to, mice, rats, hamsters, rabbits, dogs, cats, livestock, zebrafish and non-human primates.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, ²N-methylguanosine and ^{2,2}N,N-dimethylguanosine.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by a human Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

As used herein, the term “microRNA” (“miRNA”), also referred to in the art as “small temporal RNAs” (“stRNAs”), refers to a small (10-50 nucleotide) RNA which are genetically encoded (e.g., by viral, mammalian, or plant genomes) and are capable of directing or mediating RNA silencing. An “miRNA disorder” shall refer to a disease or disorder characterized by an aberrant expression or activity of an miRNA.

As used herein, the term “dual functional oligonucleotide” refers to an f-circRNA inhibiting agent having the formula T-L-μ, wherein T is an f-circRNA targeting moiety, L is a linking moiety, and μ is a miRNA recruiting moiety. As used herein, the terms “f-circRNA targeting moiety,” “targeting moiety,” “f-circRNA targeting portion” or “targeting portion” refer to a domain, portion or region of the dual functional oligonucleotide having sufficient size and sufficient complementarity to a portion or region of an f-circRNA chosen or targeted for silencing (i.e., the moiety has a sequence sufficient to capture the target f-circRNA, e.g., the back-splice junction). As used herein, the term “linking moiety” or “linking portion” refers to a domain, portion or region of the RNA-silencing agent which covalently joins or links the f-circRNA.

As used herein, the term “antisense strand” of an RNA silencing agent (e.g., an f-circRNA inhibiting agent), e.g., an siRNA or shRNA, refers to a strand that is substantially complementary to a section of about 10-50 nucleotides, e.g., about 15-30, 16-25, 18-23 or 19-22 nucleotides of the f-circRNA (e.g., of the back-splice junction). The antisense strand or first strand has sequence sufficiently complementary to the desired target f-circRNA sequence to direct target-specific silencing, e.g., complementarity sufficient to trigger the destruction of the desired target f-circRNA by the RNAi machinery or process.

The term “sense strand” or “second strand” of an RNA silencing agent, e.g., an siRNA, or shRNA, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the f-circRNA (e.g., of the back-splice junction) targeted for silencing and a miRNA* strand having sufficient complementarity to form a duplex with the miRNA strand.

As used herein, the term “guide strand” refers to a strand of an RNA silencing agent, e.g., an antisense strand of an siRNA duplex or siRNA sequence, that enters into the RISC complex and directs cleavage of the target f-circRNA.

As used herein, the term “asymmetry,” as in the asymmetry of the duplex region of an RNA silencing agent (e.g., the stem of an shRNA), refers to an inequality of bond strength or base pairing strength between the termini of the RNA silencing agent (e.g., between terminal nucleotides on a first strand or stem portion and terminal nucleotides on an opposing second strand or stem portion), such that the 5′ end of one strand of the duplex is more frequently in a transient unpaired, e.g., single-stranded, state than the 5′ end of the complementary strand. This structural difference determines that one strand of the duplex is preferentially incorporated into a RISC complex. The strand whose 5′ end is less tightly paired to the complementary strand will preferentially be incorporated into RISC and mediate RNAi.

As used herein, the term “bond strength” or “base pair strength” refers to the strength of the interaction between pairs of nucleotides (or nucleotide analogs) on opposing strands of an oligonucleotide duplex (e.g., an siRNA duplex), due primarily to H-bonding, van der Waals interactions, and the like between said nucleotides (or nucleotide analogs).

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein the term “destabilizing nucleotide” refers to a first nucleotide or nucleotide analog capable of forming a base pair with second nucleotide or nucleotide analog such that the base pair is of lower bond strength than a conventional base pair (i.e., Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide.

As used herein, the term “base pair” refers to the interaction between pairs of nucleotides (or nucleotide analogs) on opposing strands of an oligonucleotide duplex (e.g., a duplex formed by a strand of an f-circRNA inhibiting agent and a target f-circRNA sequence), due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g. certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portion of universal nucleotides typically comprises a nitrogen-containing aromatic heterocyclic moiety.

As used herein, the terms “sufficient complementarity” or “sufficient degree of complementarity” mean that an f-circRNA inhibiting agent has a sequence (e.g. in the antisense strand, f-circRNA targeting moiety or miRNA recruiting moiety) which is sufficient to bind the desired target RNA, respectively, and to trigger the RNA silencing of the target f-circRNA.

Various methodologies of the instant invention include step that involves comparing a value, level, feature, characteristic, property, etc. to a “suitable control,” referred to interchangeably herein as an “appropriate control.” A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, f-circRNA level, splice rate, biological activity, cellular characteristic or property, genotype, phenotype, etc. can be determined prior to introducing an f-circRNA inhibiting agent of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and example are illustrative only and not intended to be limiting.

I. Diagnostic and Prognostic Methods

The formation of f-circRNAs can be correlated with the presence of one or more translocation-associated diseases and/or disorders (e.g., one or more cancer- or non-cancer-associated translocation diseases or disorders). In certain embodiments, a cell or an organism having a translocation-associated disease or disorder has a greater proportion of f-circRNAs relative to circRNAs. Accordingly, detecting or monitoring the presence of f-circRNAs are important diagnostic and prognostic tools, respectively, for detecting or monitoring progression of a translocation-associated disorder and/or disease.

An exemplary method for detecting the presence or absence of f-circRNAs in a biological sample involves obtaining a biological sample from a test subject and contacting the biological sample with a compound or an agent capable of detecting f-circRNAs such that the presence of f-circRNAs are detected in the biological sample. A preferred agent for detecting f-circRNAs is a labeled nucleic acid probe capable of hybridizing to f-circRNAs. Other suitable probes for use in the diagnostic assays of the invention are described herein.

In one embodiment, the biological sample contains f-circRNAs from the test subject. A preferred biological sample is a serum sample isolated by conventional means from a subject.

In certain embodiments, f-circRNA expressed in a cell or an organism having a translocation disease or disorder undergoes exocytosis and can be detected in endocytic vesicles. Accordingly, a preferred biological sample is a lysosomal fraction obtained by conventional means from a subject.

Lysosomes are organelles ubiquitously distributed in most eukaryotic cells (lysosomes are not present in red blood cells). They are spherical particles with a diameter of 0.5-1.5 microns that have a low pH (approximately pH 5.0) and contain acid hydrolases, lipases, polysaccharidases and nucleases. By being readily obtainable from most cells, lysosomal fractions allow the facile detection of translocation-associated disorders that may be difficult to detect using traditional biopsy-based methods, e.g., for blood-based cancers or cancers in difficult to access portions of the body of a subject.

The presence of lysosomes in a sample can be determined by measuring acid phosphatase and/or β-N-acetylglucosaminidase activities using kits known in the art (e.g., from Sigma Aldrich, Catalog Numbers CS0740 and CS0780, respectively). Acid phosphatase and β-N-acetylglucosaminidase serve as lysosomal markers and will show slightly different patterns on sucrose density gradients. Lysosomal membranes can be isolated from tissue and/or cell samples by disruption of the lysosomes followed by enrichment using gradient centrifugation, e.g., step sucrose gradient centrifugation, Percoll gradient centrifugation, iodixanol gradient centrifugation or the like, using methods well-known in the art. (See, e.g., Musalkova et al. (2013) Folia Biol. 59:41; Graham (2001) Curr. Protoc. Cell Biol., chapter 3, unit 3.6, doi: 10.1002/0471143030.cb0306s07; Yamada et al. (1983) J. Biochem. 95:1155; Kawashima et al. (1998) Kidney International 54:275).

In another embodiment, the methods further involve obtaining a control biological sample from a control subject, contacting the control sample with a compound or agent capable of detecting f-circRNAs, such that the presence of f-circRNAs are detected in the biological sample, and comparing the presence of f-circRNAs in the control sample with the presence of f-circRNAs in the test sample.

The diagnostic methods described herein can furthermore be utilized to identify subjects having or at risk of developing a disease or disorder associated with a chromosomal translocation that leads to f-circRNA expression or activity, e.g., a translocation-associated disease or disorder.

The assays described herein, such as the preceding diagnostic assays or the following assays, can be utilized to identify a subject having or at risk of developing a translocation-associated disorder associated with f-circRNA expression, such as cancer. Thus, the present invention provides a method for identifying a translocation-associated disease or disorder associated with f-circRNA expression or activity in which a test sample is obtained from a subject and f-circRNA is detected, wherein the presence of f-circRNA is diagnostic for a subject having or at risk of developing a translocation-associated disease or disorder associated with f-circRNA expression or activity. As used herein, a “test sample” refers to a biological sample obtained from a subject of interest. For example, a test sample can be a biological fluid (e.g., serum), cell sample, tissue or cell fraction (e.g., a lysosomal fraction).

Furthermore, prognostic assays described herein can be used to determine whether a subject can be administered an agent, e.g., an f-circRNA inhibiting agent (e.g., an agonist, antagonist, peptidomimetic, protein, peptide, nucleic acid, small molecule, RNAi agent or other drug candidate) to treat a translocation-associated disease or disorder associated with f-circRNA expression or activity. For example, such methods can be used to determine whether a subject can be effectively treated with an agent for cancer. Thus, the present invention provides methods for determining whether a subject can be effectively treated with an agent for a translocation-associated disorder associated with f-circRNA expression or activity in which a test sample is obtained and f-circRNA expression or activity is detected (e.g., wherein the abundance of f-circRNA expression or activity is diagnostic for a subject that can be administered the agent to treat a translocation-associated disorder associated with f-circRNA expression or activity).

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a translocation-associated disease or illness involving f-circRNA.

Furthermore, any fluid, tissue, cell or cell fraction in which f-circRNA is expressed may be utilized in the prognostic assays described herein.

Monitoring the influence of f-circRNA inhibiting agents (e.g., drugs, e.g., RNAi agents) on the expression or activity of f-circRNA can be applied not only in basic drug screening, but also in clinical trials. For example, the effectiveness of an agent determined by a screening assay to decrease f-circRNA expression or downregulate f-circRNA activity, can be monitored in clinical trials of subjects exhibiting increased f-circRNA expression or upregulated f-circRNA activity. In such clinical trials, the expression or activity of f-circRNA can be used as a “read out” or markers of the phenotype of a particular cell.

For example, and not by way of limitation, f-circRNA modulation in cells by treatment with an agent (e.g., compound, drug or small molecule, e.g., an RNAi agent) which inhibits f-circRNA expression and/or activity (e.g., identified in a screening assay as described herein) can be identified. Thus, to study the effect of agents on translocation-associated disorders (e.g., disorders cancer), for example, in a clinical trial, cells can be isolated and f-circRNA prepared and analyzed for the levels of expression of f-circRNA and other markers implicated in the translocation-associated disorder, respectively. The levels of f-circRNA expression (e.g., an f-circRNA expression pattern) can be quantified by northern blot analysis or RT-PCR, as described herein, or by measuring the levels of activity of f-circRNA. In this way, the f-circRNA pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an f-circRNA inhibiting agent (e.g., an agonist, antagonist, peptidomimetic, protein, peptide, nucleic acid, small molecule, RNAi agent or other drug candidate identified by the screening assays described herein) including the steps of (i) obtaining a pre-administration sample from a subject prior to administration of the agent; (ii) detecting the level of expression or activity of f-circRNA in the pre-administration sample; (iii) obtaining one or more post-administration samples from the subject; (iv) detecting the level of expression or activity of the f-circRNA in the post-administration samples; (v) comparing the level of expression or activity of the f-circRNA in the pre-administration sample with the f-circRNA in the post administration sample or samples; and (vi) altering the administration of the agent to the subject accordingly. For example, increased administration of the agent may be desirable to decrease the expression or activity of f-circRNA to lower levels than detected, i.e., to increase the effectiveness of the agent. Alternatively, decreased administration of the agent may be desirable to increase expression or activity of f-circRNA to lower levels than detected, i.e., to decrease the effectiveness of the agent. According to such an embodiment, f-circRNA expression or activity may be used as an indicator of the effectiveness of an agent, even in the absence of an observable phenotypic response.

II. Therapeutic Methods of Treatment

The present invention provides for both prophylactic and therapeutic methods of treating a subject at risk of (or susceptible to) a disease or disorder mediated, in whole or in part, by f-circRNA. In one embodiment, the disease or disorder is a translocation-associated disorders (e.g., cancer-associated translocation disorders and/or non-cancerous translocation disorders). In a preferred embodiment, the disease or disorder is an F-circRNA-associated cancer, e.g., acute promyelocytic leukemia (APL), MLL-AF9-mediated leukemia, Ewing sarcoma, non-small cell lung carcinoma and the like.

“Treatment,” or “treating,” as used herein, is defined as the application or administration of a therapeutic agent, e.g., an f-circRNA inhibiting agent (e.g., an RNA agent or vector or transgene encoding same or a DNA blocking agent) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent, e.g., an f-circRNA inhibiting agent (e.g., an RNAi agent or vector or transgene encoding same or a DNA blocking agent). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

Another aspect of the invention pertains to methods of treating subjects therapeutically, i.e., to alter onset of symptoms of the disease or disorder. In an exemplary embodiment, the modulatory method of the invention involves contacting a cell expressing a translocation with a therapeutic agent, e.g., an f-circRNA inhibiting agent (e.g., a RNAi agent or vector or transgene encoding same or an agent that blocks DNA binding) that is specific for one or more back-splice junction target sequences (e.g., one or more sequences set forth in FIGS. 7 and 8, e.g., one or more of SEQ ID Nos:1-6 and/or fragments and/or homologs thereof), such that sequence specific interference with the f-circRNA is achieved. These methods can be performed in vitro (e.g., by culturing the cell with the agent) or, alternatively, in vivo (e.g., by administering the agent to a subject).

Therapeutic agents, e.g., f-circRNA inhibiting agents, can be tested in an appropriate animal model in which the animal expresses one or more f-circRNAs and/or complements thereof. For example, an f-circRNA inhibiting agent (e.g., RNAi agent (or expression vector or transgene encoding same) or DNA binding agent) as described herein can be used in an animal model to determine the efficacy, toxicity, or side effects of treatment with said agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

A pharmaceutical composition containing a therapeutic agent, e.g., an f-circRNA inhibiting agent (e.g., an RNAi agent or DNA binding agent) of the invention can be administered to any patient diagnosed as having or at risk for developing a translocation-associated disease or disorder, such as cancer. In one embodiment, the patient is diagnosed as having cancer, and the patient is otherwise in general good health. For example, the patient is not terminally ill, and the patient is likely to live at least two, three, five or more years following diagnosis. In another embodiment, the patient has not reached an advanced stage of the disorder or disease. In another embodiment, the patient has reached an advanced stage of the disorder or disease.

In one embodiment, a subject is administered an initial dose, and one or more maintenance doses of a therapeutic agent, e.g., an f-circRNA inhibiting agent (e.g., an RNAi agent or an agent that blocks DNA binding). The maintenance dose or doses are generally lower than the initial dose, e.g., one-half less of the initial dose. A maintenance regimen can include treating the subject with a dose or doses ranging from 0.01 μg to 1.4 mg/kg of body weight per day, e.g., 10, 1, 0.1, 0.01, 0.001, or 0.00001 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable. In one embodiment, a pharmaceutical composition includes a plurality of therapeutic agents (e.g., RNAi agents, DNA binding agents, cancer chemotherapeutics and the like). In another embodiment, the RNAi agent species has sequences that are non-overlapping and non-adjacent to another species with respect to a naturally occurring target sequence. In another embodiment, the plurality of f-circRNA inhibiting agents is specific for different regions of a back-splice junction. In another embodiment, the plurality of RNAi agents target two or more target sequences of a back-splice junction (e.g., two, three, four, five, six, or more target sequences of a back-splice junction).

Following successful treatment, it may be desirable to have the patient undergo maintenance therapy to prevent the recurrence of the disease state, wherein the compound of the invention is administered in maintenance doses, ranging from 0.01 μg to 100 g per kg of body weight (see U.S. Pat. No. 6,107,094). In certain exemplary embodiments, maintenance therapy prevents a relapse from cancer remission in a subject.

The concentration of the therapeutic agent, e.g., an f-circRNA inhibiting agent (e.g., an RNAi agent or an agent that blocks DNA binding) composition is an amount sufficient to be effective in treating or preventing a disorder or to regulate a physiological condition in humans. The concentration or amount of therapeutic agent, e.g., an f-circRNA inhibiting agent (e.g., an RNAi agent or an agent that blocks DNA binding) administered will depend on the parameters determined for the agent and the method of administration, e.g. nasal, buccal, or pulmonary. For example, nasal formulations tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a therapeutic agent, e.g., an f-circRNA inhibiting agent (e.g., an RNAi agent or an agent that blocks DNA binding) can include a single treatment or, preferably, can include a series of treatments. It will also be appreciated that the effective dosage of a therapeutic agent, e.g., an f-circRNA inhibiting agent (e.g., an RNAi agent or an agent that blocks DNA binding) for treatment may increase or decrease over the course of a particular treatment. Changes in dosage may result and become apparent from the results of diagnostic assays as described herein. For example, the subject can be monitored after administering a therapeutic agent, e.g., an f-circRNA inhibiting agent (e.g., an RNAi agent or an agent that blocks DNA binding) composition. Based on information from the monitoring, an additional amount of the therapeutic agent, e.g., an f-circRNA inhibiting agent (e.g., an RNAi agent or an agent that blocks DNA binding) composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that express a human gene translocation, e.g., a gene translocation that produces a target f-circRNA. The transgenic animal can be deficient for the corresponding f-circRNA.

III. Pharmaceutical Compositions and Methods of Administration

The invention pertains to uses of the above-described f-circRNA inhibiting agents for prophylactic and/or therapeutic treatments as described Infra. Accordingly, the f-circRNA inhibiting agent (e.g., RNAi agents and/or DNA binding agents) of the present invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds (e.g., one or more traditional chemotherapy compounds) can also be incorporated into the compositions.

A pharmaceutical composition of the invention is formulated to be compatible with its intended route of administration. Examples of routes of administration include parenteral, e.g., intravenous (IV), intradermal, subcutaneous (SC or SQ), intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The f-circRNA inhibiting agents can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

The f-circRNA inhibiting agents can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the f-circRNA inhibiting agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such f-circRNA inhibiting compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans Levels in plasma may be measured, for example, by high performance liquid chromatography.

The pharmaceutical compositions can be included in a container, pack or dispenser together with optional instructions for administration.

As defined herein, a therapeutically effective amount of an f-circRNA inhibiting agent, e.g., a RNAi agent or an agent that blocks DNA binding (i.e., an effective dosage) depends on the RNAi agent or DNA binding agent selected. For instance, if a plasmid encoding shRNA is selected, single dose amounts in the range of approximately 1 μg to 1000 mg may be administered; in some embodiments, 10, 30, 100 or 1000 μg may be administered. In some embodiments, 1-5 g of the compositions can be administered. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

The f-circRNA inhibiting agents (e.g., nucleic acid molecules) of the invention can be inserted into expression constructs, e.g., viral vectors, retroviral vectors, expression cassettes, or plasmid viral vectors, e.g., using methods known in the art, including but not limited to those described in Xia et al., (2002), Supra. Expression constructs can be delivered to a subject by, for example, inhalation, orally, intravenous injection, local administration (see U.S. Pat. No. 5,328,470) or by stereotactic injection (see e.g., Chen et al. (1994), Proc. Natl. Acad. Sci. USA, 91, 3054-3057). The pharmaceutical preparation of the delivery vector can include the vector in an acceptable diluent, or can comprise a slow release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The f-circRNA inhibiting agents of the invention can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides. Brummelkamp et al. (2002), Science, 296, 550-553; Lee et al, (2002). Supra; Miyagishi and Taira (2002), Nature Biotechnol., 20, 497-500; Paddison et al. (2002), Supra; Paul (2002), Supra; Sui (2002) Supra; Yu et al. (2002), Supra.

The expression constructs may be any construct suitable for use in the appropriate expression system and include, but are not limited to retroviral vectors, linear expression cassettes, plasmids and viral or virally-derived vectors, as known in the art. Such expression constructs may include one or more inducible promoters, RNA Pol III promoter systems such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct, Tuschl (2002), Supra.

The route of delivery can be dependent on the disorder of the patient. In certain exemplary embodiments, a subject diagnosed with a translocation-associated disorder, e.g., cancer, can be administered an f-circRNA inhibiting agent of the invention by IV or SC administration. In addition to an f-circRNA inhibiting agent of the invention, a patient can be administered a second therapy, e.g., a palliative therapy and/or disease-specific therapy, e.g., one or more chemotherapeutic agents or cancer drugs known in the art. The secondary therapy can be, for example, symptomatic (e.g., for alleviating symptoms), protective (e.g., for slowing or halting disease progression), or restorative (e.g., for reversing the disease process).

In an exemplary embodiment, one or more f-circRNA inhibiting agents are used in combination with one or more known cancer drugs. For example, one or more f-circRNA inhibiting agents can be administered to a subject prior to treatment with one or more known cancer drugs, concomitant with treatment with one or more known cancer drugs, or after treatment with one or more cancer drugs. In certain embodiments, an f-circRNA inhibiting agent can potentiate the anti-cancer activity of one or more known cancer drugs.

In an exemplary embodiment, one or more f-circRNA inhibiting agents are used to treat a subject having a cancer that is resistant to treatment with one or more cancer drugs known in the art. In other embodiments, an f-circRNA inhibiting agent is used during remission to prevent recurrence of the cancer from full remission. In other embodiments, an f-circRNA inhibiting agent is used to treat a cancer in partial remission.

In general, an f-circRNA inhibiting agent of the invention can be administered by any suitable method. As used herein, topical delivery can refer to the direct application of an f-circRNA inhibiting agent to any surface of the body, including the eye, a mucous membrane, surfaces of a body cavity, or to any internal surface. Formulations for topical administration may include transdermal patches, ointments, lotions, creams, gels, drops, sprays, and liquids. Conventional pharmaceutical carriers, aqueous, powder or oily bases, thickeners and the like may be necessary or desirable. Topical administration can also be used as a means to selectively deliver the f-circRNA inhibiting agent to the epidermis or dermis of a subject, or to specific strata thereof, or to an underlying tissue.

Compositions for intrathecal or intraventricular administration may include sterile aqueous solutions which may also contain buffers, diluents and other suitable additives. Compositions for intrathecal or intraventricular administration preferably do not include a transfection reagent or an additional lipophilic moiety besides, for example, the lipophilic moiety attached to the f-circRNA inhibiting agent.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

An f-circRNA inhibiting agent of the invention can be administered to a subject by pulmonary delivery. Pulmonary delivery compositions can be delivered by inhalation of a dispersion so that the composition within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self-contained. Dry powder dispersion devices, for example, deliver drugs that may be readily formulated as dry powders. An f-circRNA inhibiting agent composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The types of pharmaceutical excipients that are useful as carriers include stabilizers such as Human Serum Albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-β-cyclodextrin; and polysaccharides, such as raffinose, maltodextrins, dextrans, and the like; alditols, such as mannitol, xylitol, and the like. A preferred group of carbohydrates includes lactose, trehalose, raffinose maltodextrins, and mannitol. Suitable polypeptides include aspartame Amino acids include alanine and glycine, with glycine being preferred.

Suitable pH adjusters or buffers include organic salts prepared from organic acids and bases, such as sodium citrate, sodium ascorbate, and the like; sodium citrate is preferred.

An f-circRNA inhibiting agent of the invention can be administered by oral and nasal delivery. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily. In one embodiment, an f-circRNA inhibiting agent administered by oral or nasal delivery has been modified to be capable of traversing the blood-brain barrier.

In one embodiment, unit doses or measured doses of a composition that include f-circRNA inhibiting agents are dispensed by an implanted device. The device can include a sensor that monitors a parameter within a subject. For example, the device can include a pump, such as an osmotic pump and, optionally, associated electronics.

An f-circRNA inhibiting agent can be packaged in a viral natural capsid or in a chemically or enzymatically produced artificial capsid or structure derived therefrom.

IV. Reagents

In certain exemplary embodiments, f-circRNA inhibiting agents of the invention (e.g., siRNAs, shRNAs or DNA blocking agents) are designed to target a back-splice junction of an f-circRNA.

A. siRNA Design

In some embodiments, siRNAs are designed as follows. First, a portion of the target (e.g., a back-splice junction of an f-circRNA) is selected. Cleavage of f-circRNA should eliminate one or more f-circRNA activities. Preferably, the target sequence (and corresponding sense strand) includes about 20 to 25 nucleotides, e.g., 20, 21, 22, 23, 24 or 25 nucleotides. More preferably, the portion (and corresponding sense strand) includes 21, 22 or 23 nucleotides. The skilled artisan will appreciate, however, that siRNAs having a length of less than 19 nucleotides or greater than 25 nucleotides can also function to mediate RNAi. Accordingly, siRNAs of such length are also within the scope of the instant invention provided that they retain the ability to mediate RNAi. Longer RNAi agents have been demonstrated to elicit an interferon or Protein Kinase R (PKR) response in certain mammalian cells which may be undesirable. Preferably, the RNAi agents of the invention do not elicit a PKR response (i.e., are of a sufficiently short length). However, longer RNAi agents may be useful, for example, in cell types incapable of generating a PRK response or in situations where the PKR response has been down-regulated or dampened by alternative means.

The sense strand sequence is designed such that the target sequence is essentially in the middle of the strand. Moving the target sequence to an off-center position may, in some instances, reduce efficiency of cleavage by the siRNA. Such compositions, i.e., less efficient compositions, may be desirable for use if off-silencing of the f-circRNA is detected.

The antisense strand is routinely the same length as the sense strand and includes complementary nucleotides. In one embodiment, the strands are fully complementary, i.e., the strands are blunt-ended when aligned or annealed. In another embodiment, the strands comprise align or anneal such that 1-, 2- or 3-nucleotide overhangs are generated, i.e., the 3′ end of the sense strand extends 1, 2 or 3 nucleotides further than the 5′ end of the antisense strand and/or the 3′ end of the antisense strand extends 1, 2 or 3 nucleotides further than the 5′ end of the sense strand. Overhangs can comprise (or consist of) nucleotides corresponding to the target f-circRNA (or complement thereof). Alternatively, overhangs can comprise (or consist of) deoxyribonucleotides, for example dTs, or nucleotide analogs, or other suitable non-nucleotide material.

To facilitate entry of the antisense strand into RISC (and thus increase or improve the efficiency of target cleavage and silencing), the base pair strength between the 5′ end of the sense strand and 3′ end of the antisense strand can be altered, e.g., lessened or reduced, as described in detail in U.S. Pat. Nos. 7,459,547, 7,772,203 and 7,732,593, entitled “Methods and Compositions for Controlling Efficacy of RNA Silencing” (filed Jun. 2, 2003) and U.S. Pat. Nos. 8,309,704, 7,750,144, 8,304,530, 8,329,892 and 8,309,705, entitled “Methods and Compositions for Enhancing the Efficacy and Specificity of RNAi” (filed Jun. 2, 2003), the contents of which are incorporated in their entirety by this reference. In one embodiment of these aspects of the invention, the base-pair strength is less due to fewer G:C base pairs between the 5′ end of the first or antisense strand and the 3′ end of the second or sense strand than between the 3′ end of the first or antisense strand and the 5′ end of the second or sense strand. In another embodiment, the base pair strength is less due to at least one mismatched base pair between the 5′ end of the first or antisense strand and the 3′ end of the second or sense strand. In certain exemplary embodiments, the mismatched base pair is selected from the group consisting of G:A, C:A, C:U, G:G, A:A, C:C and U:U. In another embodiment, the base pair strength is less due to at least one wobble base pair, e.g., G:U, between the 5′ end of the first or antisense strand and the 3′ end of the second or sense strand. In another embodiment, the base pair strength is less due to at least one base pair comprising a rare nucleotide, e.g., inosine (I). In certain exemplary embodiments, the base pair is selected from the group consisting of an I:A, I:U and I:C. In yet another embodiment, the base pair strength is less due to at least one base pair comprising a modified nucleotide. In certain exemplary embodiments, the modified nucleotide comprises a nucleobase selected from the group consisting of 2-aminopurine and 2,6-diaminopurine.

To validate the effectiveness by which siRNAs destroys an f-circRNA, the siRNA can be incubated with a vector expressing circularizing exons in a Drosophila-based in vitro expression system. Radiolabeled with ³²P, newly synthesized f-circRNAs are detected autoradiographically on an agarose gel. The presence of cleaved f-circRNAs indicates siRNA efficacy. Suitable controls include omission of siRNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

Sites of siRNA-f-circRNA complementation within the back-splice junction are selected which result in optimal f-circRNA specificity and maximal f-circRNA cleavage.

B. RNAi Agents

The present invention includes siRNA molecules designed, for example, as described above. The siRNA molecules of the invention can be chemically synthesized, or can be transcribed in vitro from a DNA template, or in vivo from e.g., shRNA, or by using recombinant human DICER enzyme, to cleave in vitro transcribed dsRNA templates into pools of 20-, 21- or 23-bp duplex RNA mediating RNAi. The siRNA molecules can be designed using any method known in the art.

In one aspect, instead of the RNAi agent being an interfering ribonucleic acid, e.g., an siRNA or shRNA as described above, the RNAi agent can encode an interfering ribonucleic acid, e.g., an shRNA, as described above. In other words, the RNAi agent can be a transcriptional template of the interfering ribonucleic acid. Thus, RNAi agents of the present invention can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21-23 nucleotides (Brummelkamp et al., 2002; Lee et al., 2002, Supra; Miyagishi et al., 2002; Paddison et al., 2002, Supra; Paul et al., 2002, Supra; Sui et al., 2002 Supra; Yu et al., 2002, Supra. More information about shRNA design and use can be found on the internet at the following addresses: katandin.cshl.org:9331/RNAi/docs/BseRI-BamHI_Strategy.pdf and katandin.c shl.org:9331/RNAi/docs/Web_version_of_PCR_strategy1.pdf).

Expression constructs of the present invention include any construct suitable for use in the appropriate expression system and include, but are not limited to, retroviral vectors, linear expression cassettes, plasmids and viral or virally-derived vectors, as known in the art. Such expression constructs can include one or more inducible promoters, RNA Pol III promoter systems such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct. (Tuschl, T., 2002, Supra).

Synthetic siRNAs can be delivered into cells by methods known in the art, including cationic liposome transfection and electroporation. To obtain longer term suppression of the target f-circRNA and to facilitate delivery under certain circumstances, one or more siRNAs can be expressed within cells from recombinant DNA constructs. Such methods for expressing siRNA duplexes within cells from recombinant DNA constructs to allow longer-term target suppression in cells are known in the art, including mammalian Pol III promoter systems (e.g., H1 or U6/snRNA promoter systems (Tuschl, T., 2002, Supra) capable of expressing functional double-stranded siRNAs; (Bagella et al., 1998; Lee et al., 2002, Supra; Miyagishi et al., 2002, Supra; Paul et al., 2002, Supra; Yu et al., 2002), Supra; Sui et al., 2002, Supra). Transcriptional termination by RNA Pol III occurs at runs of four consecutive T residues in the DNA template, providing a mechanism to end the siRNA transcript at a specific sequence. The siRNA is complementary to the sequence of the target f-circRNA in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target f-circRNAs (Bagella et al., 1998; Lee et al., 2002, Supra; Miyagishi et al., 2002, Supra; Paul et al., 2002, Supra; Yu et al., 2002), Supra; Sui et al., 2002, Supra). Constructs containing siRNA sequence under the control of T7 promoter also make functional siRNAs when cotransfected into the cells with a vector expressing T7 RNA polymerase (Jacque et al., 2002, Supra). A single construct may contain multiple sequences coding for siRNAs, such as multiple regions of the gene encoding an f-circRNA or a complement thereof, and can be driven, for example, by separate PolIII promoter sites.

Animal cells express a range of noncoding RNAs of approximately 22 nucleotides termed micro RNA (miRNAs) which can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with sequence complementary to the target f-circRNA, a vector construct that expresses the engineered precursor can be used to produce siRNAs to initiate RNAi against specific f-circRNA targets in mammalian cells (Zeng et al., 2002, supra). When expressed by DNA vectors containing polymerase III promoters, micro-RNA designed hairpins can silence gene expression (McManus et al., 2002, supra). MicroRNAs targeting polymorphisms may also be useful for blocking translation of mutant proteins, in the absence of siRNA-mediated gene-silencing. Such applications may be useful in situations, for example, where a designed siRNA caused off-target silencing of wild type protein.

Viral-mediated delivery mechanisms can also be used to induce specific silencing of targeted f-circRNAs through expression of siRNA, for example, by generating recombinant adenoviruses harboring siRNA under RNA Pol II promoter transcription control (Xia et al., 2002, supra). Infection of HeLa cells by these recombinant adenoviruses allows for diminished target f-circRNA expression. Injection of the recombinant adenovirus vectors into transgenic mice expressing the target genes of the siRNA results in in vivo reduction of target f-circRNA expression. Id. In an animal model, whole-embryo electroporation can efficiently deliver synthetic siRNA into post-implantation mouse embryos (Calegari et al., 2002). In adult mice, efficient delivery of siRNA can be accomplished by “high-pressure” delivery technique, a rapid injection (within 5 seconds) of a large volume of siRNA containing solution into animal via the tail vein (Liu et al., 1999, supra; McCaffrey et al., 2002, supra; Lewis et al., 2002. Nanoparticles and liposomes can also be used to deliver siRNA into animals. In certain exemplary embodiments, recombinant adeno-associated viruses (rAAVs) and their associated vectors can be used to deliver one or more siRNAs into cells (US Patent Applications 2014/0296486, 2010/0186103, 2008/0269149, 2006/0078542 and 2005/0220766).

The nucleic acid compositions of the invention include both unmodified siRNAs and modified siRNAs as known in the art, such as crosslinked siRNA derivatives or derivatives having non nucleotide moieties linked, for example to their 3′ or 5′ ends. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Engineered RNA precursors, introduced into cells or whole organisms as described herein, will lead to the production of a desired siRNA molecule. Such an siRNA molecule will then associate with endogenous protein components of the RNAi pathway to bind to and target a specific f-circRNA sequence for cleavage and destruction. In this fashion, the f-circRNA to be targeted by the siRNA generated from the engineered RNA precursor will be depleted from the cell or organism, leading to a decrease in the concentration of the protein encoded by that f-circRNA in the cell or organism. The RNA precursors are typically nucleic acid molecules that individually encode either one strand of a dsRNA or encode the entire nucleotide sequence of an RNA hairpin loop structure.

The nucleic acid compositions of the invention can be unconjugated or can be conjugated to another moiety, such as a nanoparticle, to enhance a property of the compositions, e.g., a pharmacokinetic parameter such as absorption, efficacy, bioavailability and/or half-life. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

The nucleic acid molecules of the present invention can also be labeled using any method known in the art. For instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P or other appropriate isotope.

Moreover, because RNAi is believed to progress via at least one single-stranded RNA intermediate, the skilled artisan will appreciate that ss-siRNAs (e.g., the antisense strand of a ds-siRNA) can also be designed (e.g., for chemical synthesis) generated (e.g., enzymatically generated) or expressed (e.g., from a vector or plasmid) as described herein and utilized according to the claimed methodologies. Moreover, in invertebrates, RNAi can be triggered effectively by long dsRNAs (e.g., dsRNAs about 100-1000 nucleotides in length, preferably about 200-500, for example, about 250, 300, 350, 400 or 450 nucleotides in length) acting as effectors of RNAi. (Brondani et al., Proc Natl Acad Sci USA. 2001 Dec. 4; 98(25):14428-33. Epub 2001 Nov. 27.)

C. Anti-f-circRNAs RNA Inhibiting Agents

The present invention features anti-f-circRNAs RNA inhibiting agents (e.g., siRNA and shRNAs), methods of making said RNA inhibiting agents, and methods (e.g., research and/or therapeutic methods) for using said improved RNA inhibiting agents (or portions thereof) for RNA inhibiting of one or more f-circRNAs. The RNA inhibiting agents comprise an antisense strand (or portions thereof), wherein the antisense strand has sufficient complementary to a back-splice junction an f-circRNAs to mediate an RNA-mediated inhibition (e.g. RNAi).

a) Design of Anti-f-circRNA shRNA Molecules

In certain featured embodiments, the instant invention provides shRNAs capable of mediating f-circRNA inhibition with enhanced selectivity. In contrast to siRNAs, shRNAs mimic the natural precursors of micro RNAs (miRNAs) and enter at the top of the gene silencing pathway. For this reason, shRNAs are believed to mediate gene silencing more efficiently by being fed through the entire natural gene silencing pathway.

miRNAs are noncoding RNAs of approximately 22 nucleotides which can regulate gene expression at the post-transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof. Naturally-occurring miRNA precursors (pre-miRNA) have a single strand that forms a duplex stem including two portions that are generally complementary, and a loop, that connects the two portions of the stem. In typical pre-miRNAs, the stem includes one or more bulges, e.g., extra nucleotides that create a single nucleotide “loop” in one portion of the stem, and/or one or more unpaired nucleotides that create a gap in the hybridization of the two portions of the stem to each other. Short hairpin RNAs, or engineered RNA precursors, of the invention are artificial constructs based on these naturally occurring pre-miRNAs, but which are engineered to deliver desired f-circRNA inhibiting agents (e.g., siRNAs or shRNAs of the invention). By substituting the stem sequences of the pre-miRNA with sequence complementary to the target f-circRNA, a shRNA is formed. The shRNA is processed by the entire gene silencing pathway of the cell, thereby efficiently mediating RNAi.

In shRNAs (or engineered precursor RNAs) of the instant invention, one portion of the duplex stem is a nucleic acid sequence that is complementary (or antisense) to the f-circRNA back-splice junction target sequence. Preferably, one strand of the stem portion of the shRNA is sufficiently complementary (e.g., antisense) to a target RNA (e.g., f-circRNA) sequence to mediate degradation or cleavage of said target RNA via RNA interference (RNAi). Thus, engineered RNA precursors include a duplex stem with two portions and a loop connecting the two stem portions. The antisense portion can be on the 5′ or 3′ end of the stem. The stem portions of a shRNA are preferably about 15 to about 50 nucleotides in length. Preferably the two stem portions are about 18 or 19 to about 21, 22, 23, 24, 25, 30, 35, 37, 38, 39, or 40 or more nucleotides in length. In preferred embodiments, the length of the stem portions should be 21 nucleotides or greater. When used in mammalian cells, the length of the stem portions should be less than about 30 nucleotides to avoid provoking non-specific responses like the interferon pathway. In non-mammalian cells, the stem can be longer than 30 nucleotides. In fact, the stem can include much larger sections complementary to the target f-circRNA (up to, and including the entire f-circRNA).

The two portions of the duplex stem must be sufficiently complementary to hybridize to form the duplex stem. Thus, the two portions can be, but need not be, fully or perfectly complementary. In addition, the two stem portions can be the same length, or one portion can include an overhang of 1, 2, 3, or 4 nucleotides. The overhanging nucleotides can include, for example, uracils (Us), e.g., all Us. The loop in the shRNAs or engineered RNA precursors may differ from natural pre-miRNA sequences by modifying the loop sequence to increase or decrease the number of paired nucleotides, or replacing all or part of the loop sequence with a tetraloop or other loop sequences. Thus, the loop in the shRNAs or engineered RNA precursors can be 2, 3, 4, 5, 6, 7, 8, 9, or more, e.g., 15 or 20, or more nucleotides in length.

The loop in the shRNAs or engineered RNA precursors may differ from natural pre-miRNA sequences by modifying the loop sequence to increase or decrease the number of paired nucleotides, or replacing all or part of the loop sequence with a tetraloop or other loop sequences. Thus, the loop portion in the shRNA can be about 2 to about 20 nucleotides in length, i.e., about 2, 3, 4, 5, 6, 7, 8, 9, or more, e.g., 15 or 20, or more nucleotides in length. A preferred loop consists of or comprises a “tetraloop” sequences. Exemplary tetraloop sequences include, but are not limited to, the sequences GNRA, where N is any nucleotide and R is a purine nucleotide, GGGG, and UUUU.

According to one embodiment of the invention, an shRNA was designed that functioned as an f-circRNA inhibiting agent having a sequence of 30 base pairs (bp) (e.g., approximately 15 bp up-stream and approximately 15 bp down-stream of the back-splice junction) and provided as input for the selector tool. As an output, a list of several possible shRNAs of 21 bp in length, each with different starting positions were used. Sequences were listed according to their “intrinsic score,” which took into account the performance in targeting the specific sequence, and the ability to be cloned into a specific vector (i.e., the pLKO.1 vector—See Examples, Infra). The higher the intrinsic score, the better the chance of cloning a high performing shRNA. According to the starting point of the input sequence and the intrinsic score, sequences that had less than 14 bp shifted to one side of the junction were selected. By choosing these sequences, shRNAs that might target linear transcripts other than the f-circRNA were excluded. Selected sequences were analyzed using NCBI's BLAST program in order to predict possible off-target effects. Sequences with 100% similarity to unrelated transcripts were excluded.

In certain embodiments, shRNAs of the invention include the sequences of a desired siRNA molecule described herein. In other embodiments, the sequence of the antisense portion of a shRNA can be designed essentially as described above or generally by selecting an 18, 19, 20, 21 nucleotide, or longer, sequence from within the target RNA (e.g., f-circRNA). In general, the sequence can be selected from any portion of the target RNA (e.g., f-circRNA) including an intronic region, the 5′ UTR (untranslated region), coding sequence, or 3′ UTR, provided said portion is distant from the site of the gain-of-function mutation. In a particularly preferred embodiment, the sequence is a back-splice junction of an f-circRNA. This sequence can optionally follow immediately after a region of the target f-circRNA containing two adjacent AA nucleotides. The last two nucleotides of the nucleotide sequence can be selected to be UU. This 21 or so nucleotide sequence is used to create one portion of a duplex stem in the shRNA. This sequence can replace a stem portion of a wild-type pre-miRNA sequence, e.g., enzymatically, or is included in a complete sequence that is synthesized. For example, one can synthesize DNA oligonucleotides that encode the entire stem-loop engineered RNA precursor, or that encode just the portion to be inserted into the duplex stem of the precursor, and using restriction enzymes to build the engineered RNA precursor construct, e.g., from a wild-type pre-miRNA.

Engineered RNA precursors include in the duplex stem the 21-22 or so nucleotide sequences of the siRNA or siRNA-like duplex desired to be produced in vivo. Thus, the stem portion of the engineered RNA precursor includes at least 18 or 19 nucleotide pairs corresponding to the sequence of an f-circRNA target whose expression is to be reduced or inhibited. The two 3′ nucleotides flanking this region of the stem are chosen so as to maximize the production of the siRNA from the engineered RNA precursor and to maximize the efficacy of the resulting siRNA in targeting the corresponding f-circRNA for destruction by RNAi in vivo and in vitro.

In certain embodiments, shRNAs of the invention include miRNA sequences, optionally end-modified miRNA sequences, to enhance entry into RISC. The miRNA sequence can be similar or identical to that of any naturally occurring miRNA (see e.g. The miRNA Registry; Griffiths-Jones S, Nuc. Acids Res., 2004). Over one thousand natural miRNAs have been identified to date and together they are thought to comprise about 1% of all predicted genes in the genome. Many natural miRNAs are clustered together in the introns of pre-mRNAs and can be identified in silico using homology-based searches (Pasquinelli et al., 2000; Lagos-Quintana et al., 2001; Lau et al., 2001; Lee and Ambros, 2001) or computer algorithms (e.g. MiRScan, MiRSeeker) that predict the capability of a candidate miRNA gene to form the stem loop structure of a pri-mRNA (Grad et al., Mol. Cell., 2003; Lim et al., Genes Dev., 2003; Lim et al., Science, 2003; Lai E C et al., Genome Bio., 2003). An online registry provides a searchable database of all published miRNA sequences (The miRNA Registry at the Sanger Institute website; Griffiths-Jones S, Nuc. Acids Res., 2004). Exemplary, natural miRNAs include lin-4, let-7, miR-10, mirR-15, miR-16, miR-168, miR-175, miR-196 and their homologs, as well as other natural miRNAs from humans and certain model organisms including Drosophila melanogaster, Caenorhabditis elegans, zebrafish, Arabidopsis thalania, Mus musculus, and Rattus norvegicus as described in International PCT Publication No. WO 03/029459.

Naturally-occurring miRNAs are expressed by endogenous genes in vivo and are processed from a hairpin or stem-loop precursor (pre-miRNA or pri-miRNAs) by Dicer or other RNAses (Lagos-Quintana et al., Science, 2001; Lau et al., Science, 2001; Lee and Ambros, Science, 2001; Lagos-Quintana et al., Curr. Biol., 2002; Mourelatos et al., Genes Dev., 2002; Reinhart et al., Science, 2002; Ambros et al., Curr. Biol., 2003; Brennecke et al., 2003; Lagos-Quintana et al., RNA, 2003; Lim et al., Genes Dev., 2003; Lim et al., Science, 2003). miRNAs can exist transiently in vivo as a double-stranded duplex, but only one strand is taken up by the RISC complex to direct gene silencing. Certain miRNAs, e.g., plant miRNAs, have perfect or near-perfect complementarity to their target mRNAs and, hence, direct cleavage of the target mRNAs. Other miRNAs have less than perfect complementarity to their target mRNAs and, hence, direct translational repression of the target mRNAs. The degree of complementarity between an miRNA and its target mRNA is believed to determine its mechanism of action. For example, perfect or near-perfect complementarity between a miRNA and its target mRNA is predictive of a cleavage mechanism (Yekta et al., Science, 2004), whereas less than perfect complementarity is predictive of a translational repression mechanism.

b) Design of Anti-f-circRNA siRNA Molecules

An siRNA molecule of the invention is a duplex consisting of a sense strand and complementary antisense strand, the antisense strand having sufficient complementary to an f-circRNA (e.g., to a back-splice junction (e.g., spanning a back-splice junction comprising a portion of exons of two genes)) to mediate RNAi. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is sufficiently complementary to a target region. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to a target sequence, and the other strand is identical or substantially identical to the first strand.

Generally, siRNAs can be designed by using any method known in the art, for instance, by using the following protocol:

1. The siRNA should be specific for a target sequence, e.g., a target sequence set forth in an f-circRNA (e.g., a back-splice junction region). The first strand should be complementary to the target sequence, and the other strand should be substantially complementary to the first strand. In one embodiment, the target sequence is encoded in a back-splice junction of one or more f-circRNAs. Exemplary target sequences correspond to a back-splice junction that includes exonic regions corresponding to two genes. A sense strand is designed based on the target sequence. Further, siRNAs with lower G/C content (35-55%) may be more active than those with G/C content higher than 55%. Thus in one embodiment, the invention includes nucleic acid molecules having 35-55% G/C content.

2. The sense strand of the siRNA is designed based on the sequence of the selected target site. Preferably the sense strand includes about 19 to 25 nucleotides, e.g., 19, 20, 21, 22, 23, 24 or 25 nucleotides. More preferably, the sense strand includes 21, 22 or 23 nucleotides. The skilled artisan will appreciate, however, that siRNAs having a length of less than 19 nucleotides or greater than 25 nucleotides can also function to mediate RNAi. Accordingly, siRNAs of such length are also within the scope of the instant invention provided that they retain the ability to mediate RNAi. Longer RNA silencing agents have been demonstrated to elicit an interferon or Protein Kinase R (PKR) response in certain mammalian cells which may be undesirable. Preferably the RNA silencing agents of the invention do not elicit a PKR response (i.e., are of a sufficiently short length). However, longer RNA silencing agents may be useful, for example, in cell types incapable of generating a PRK response or in situations where the PKR response has been down-regulated or dampened by alternative means.

The siRNA molecules of the invention have sufficient complementarity with the target sequence such that the siRNA can mediate RNAi. In general, siRNA containing nucleotide sequences sufficiently identical to a target sequence portion of the f-circRNA to effect RISC-mediated cleavage of the target f-circRNA are preferred. Accordingly, in a preferred embodiment, the sense strand of the siRNA is designed have to have a sequence sufficiently identical to a portion of the target. For example, the sense strand may have 100% identity to the target site. However, 100% identity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% identity, between the sense strand and the target RNA sequence is preferred. The invention has the advantage of being able to tolerate certain sequence variations to enhance efficiency and specificity of RNAi. In one embodiment, the sense strand has 4, 3, 2, 1, or 0 mismatched nucleotide(s) with a target region, such as a target region that differs by at least one base pair between an f-circRNA and an mRNA or a circRNA. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=number of identical positions/total number of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

3. The antisense or guide strand of the siRNA is routinely the same length as the sense strand and includes complementary nucleotides. In one embodiment, the guide and sense strands are fully complementary, i.e., the strands are blunt-ended when aligned or annealed. In another embodiment, the strands of the siRNA can be paired in such a way as to have a 3′ overhang of 1 to 4, e.g., 2, nucleotides. Overhangs can comprise (or consist of) nucleotides corresponding to the target f-circRNA (or complement thereof). Alternatively, overhangs can comprise (or consist of) deoxyribonucleotides, for example dTs, or nucleotide analogs, or other suitable non-nucleotide material. Thus in another embodiment, the nucleic acid molecules may have a 3′ overhang of 2 nucleotides, such as TT. The overhanging nucleotides may be either RNA or DNA. As noted above, it is desirable to choose a target region wherein the mutant:wild type mismatch is a purine:purine mismatch.

4. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.

5. Select one or more sequences that meet your criteria for evaluation.

Further general information about the design and use of siRNA may be found in “The siRNA User Guide,” available at The Max-Plank-Institut fur Biophysikalishe Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (T_{M}) of the hybrid, where T_{M} is determined according to the following equations. For hybrids less than 18 base pairs in length, T_{M} (° C.)=2(# of A+T bases)+4(# of G+C bases). For hybrids between 18 and 49 base pairs in length, T_{M} (° C.)=81.5+16.6(log 10[Na+])+0.41(% G+C)-(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

6. To validate the effectiveness by which siRNAs destroy target RNAs (e.g., f-circRNA), the siRNA may be incubated with target cDNA (e.g., f-circRNA cDNA) in a Drosophila-based in vitro f-circRNA expression system. Radiolabeled with ³²P, newly synthesized target RNAs (e.g., f-circRNA) are detected autoradiographically on an agarose gel. The presence of cleaved target RNA indicates RNA nuclease activity. Suitable controls include omission of siRNA and use of non-target f-circRNA cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

Anti-f-circRNA siRNAs may be designed to target any of the target sequences described supra. Said siRNAs comprise an antisense strand which is sufficiently complementary with the target sequence to mediate silencing of the target sequence. In certain embodiments, the an f-circRNA inhibiting agent is an siRNA or an shRNA.

Sites of siRNA-f-circRNA complementation are selected which result in optimal f-circRNA specificity and maximal f-circRNA cleavage.

c) Dual Functional Oligonucleotide Tethers

In other embodiments, an f-circRNA inhibiting agent of the present invention includes dual functional oligonucleotide tethers useful for the intercellular recruitment of an miRNA. Animal cells express a range of miRNAs, noncoding RNAs of approximately 22 nucleotides which can regulate gene expression at the post transcriptional or translational level. By binding a miRNA bound to RISC and recruiting it to a target f-circRNA, a dual functional oligonucleotide tether can repress the expression of genes involved e.g., in the arteriosclerotic process. The use of oligonucleotide tethers offer several advantages over existing techniques to repress the expression of a particular gene. First, the methods described herein allow an endogenous molecule (often present in abundance), an miRNA, to mediate RNA silencing. Accordingly, the methods described herein obviate the need to introduce foreign molecules (e.g., siRNAs) to mediate RNA silencing. Second, the RNA-silencing agents and, in particular, the linking moiety (e.g., oligonucleotides such as the 2′-O-methyl oligonucleotide), can be made stable and resistant to nuclease activity. As a result, the tethers of the present invention can be designed for direct delivery, obviating the need for indirect delivery (e.g. viral) of a precursor molecule or plasmid designed to make the desired agent within the cell. Third, tethers and their respective moieties, can be designed to conform to specific f-circRNA sites and specific miRNAs. The designs can be cell and gene product specific. As a result, these methods of RNA silencing are highly regulatable.

The dual functional oligonucleotide tethers (“tethers”) of the invention are designed such that they recruit miRNAs (e.g., endogenous cellular miRNAs) to a target f-circRNA so as to inhibit the f-circRNA. In preferred embodiments, the tethers have the formula T-L-μ, wherein T is an f-circRNA targeting moiety, L is a linking moiety, and μ is an miRNA recruiting moiety. Any one or more moiety may be double stranded. Preferably, however, each moiety is single stranded.

Moieties within the tethers can be arranged or linked (in the 5′ to 3′ direction) as depicted in the formula T-L-μ (i.e., the 3′ end of the targeting moiety linked to the 5′ end of the linking moiety and the 3′ end of the linking moiety linked to the 5′ end of the miRNA recruiting moiety). Alternatively, the moieties can be arranged or linked in the tether as follows: μ-T-L (i.e., the 3′ end of the miRNA recruiting moiety linked to the 5′ end of the linking moiety and the 3′ end of the linking moiety linked to the 5′ end of the targeting moiety).

The f-circRNA targeting moiety, as described above, is capable of capturing a specific target f-circRNA. According to the invention, expression of the target f-circRNA is undesirable, and, thus, repression of the f-circRNA is desired. The f-circRNA targeting moiety should be of sufficient size to effectively bind the target f-circRNA (e.g., the back-splice junction). The length of the targeting moiety will vary greatly depending, in part, on the length of the target f-circRNA (e.g., the back-splice junction) and the degree of complementarity between the target f-circRNA (e.g., the back-splice junction) and the targeting moiety. In various embodiments, the targeting moiety is less than about 200, 100, 50, 30, 25, 20, 19, 18, 17, 16, 15, 14, 13, 12, 11, 10, 9, 8, 7, 6, or 5 nucleotides in length. In a particular embodiment, the targeting moiety is about 15 to about 25 nucleotides in length.

The f-circRNA recruiting moiety, as described above, is capable of associating with a miRNA. According to the invention, the miRNA may be any miRNA capable of repressing the target f-circRNA.

The linking moiety is any agent capable of linking the targeting moieties such that the activity of the targeting moieties is maintained. Linking moieties are preferably oligonucleotide moieties comprising a sufficient number of nucleotides such that the targeting agents can sufficiently interact with their respective targets. Linking moieties have little or no sequence homology with cellular circRNA, mRNA or miRNA sequences. Exemplary linking moieties include one or more 2′-O-methylnucleotides, e.g., 2′-β-methyladenosine, 2′-O-methylthymidine, 2′-O-methylguanosine or 2′-O-methyluridine.

D. Modified f-circRNA Silencing Agents

In certain aspects of the invention, an f-circRNA inhibiting agent (or any portion thereof) of the invention as described supra may be modified such that the activity of the agent is further improved. For example, an f-circRNA inhibiting agent described in herein may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

In certain embodiments, siRNA compounds are provided having one or any combination of the following properties: (1) fully chemically-stabilized (i.e., no unmodified 2′-OH residues); (2) asymmetry; (3) 11-16 base pair duplexes; (4) alternating pattern of chemically-modified nucleotides (e.g., 2′-fluoro and 2′-methoxy modifications); and (5) single-stranded, fully phosphorothioated tails of 5-8 bases. In certain embodiments, the siRNA compounds described herein can be conjugated to a variety of targeting agents, including, but not limited to, cholesterol, DHA, phenyltropanes, cortisol, vitamin A, vitamin D, GalNac, and gangliozides.

1) Modifications to Enhance Target Discrimination

In certain embodiments, an f-circRNA inhibiting agent of the invention may be substituted with a destabilizing nucleotide to enhance single nucleotide target discrimination (see U.S. application Ser. No. 11/698,689, filed Jan. 25, 2007 and U.S. Provisional Application No. 60/762,225 filed Jan. 25, 2006, both of which are incorporated herein by reference). Such a modification may be sufficient to abolish the specificity of the f-circRNA inhibiting agent for a non-target RNA (e.g., mRNA, circRNA or the like), without appreciably affecting the specificity of the f-circRNA inhibiting agent for a target f-circRNA.

In preferred embodiments, the f-circRNA inhibiting agents of the invention are modified by the introduction of at least one universal nucleotide in the antisense strand thereof. Universal nucleotides comprise base portions that are capable of base pairing indiscriminately with any of the four conventional nucleotide bases (e.g. A, G, C, U). A universal nucleotide is preferred because it has relatively minor effect on the stability of the RNA duplex or the duplex formed by the guide strand of the RNA silencing agent and the target f-circRNA. Exemplary universal nucleotide include those having an inosine base portion or an inosine analog base portion selected from the group consisting of deoxyinosine (e.g. 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In particularly preferred embodiments, the universal nucleotide is an inosine residue or a naturally occurring analog thereof.

In certain embodiments, the f-circRNA inhibiting agents of the invention are modified by the introduction of at least one destabilizing nucleotide within 5 nucleotides from a specificity-determining nucleotide (i.e., the nucleotide which recognizes the disease-related polymorphism). For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destablilizing nucleotide and the specificity-determining nucleotide). In f-circRNA inhibiting agents having two strands or strand portions (e.g. siRNAs and shRNAs), the destabilizing nucleotide may be introduced in the strand or strand portion that does not contain the specificity-determining nucleotide. In preferred embodiments, the destabilizing nucleotide is introduced in the same strand or strand portion that contains the specificity-determining nucleotide.

2) Modifications to Enhance Efficacy and Specificity

In certain embodiments, the f-circRNA inhibiting agents of the invention may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of the siRNA (e.g., a siRNA designed using the methods of the invention or an siRNA produced from a shRNA) into RISC in favor of the sense strand, such that the antisense strand preferentially guides cleavage of a target f-circRNA, and thus increasing or improving the efficiency of target cleavage and silencing. Preferably the asymmetry of an f-circRNA inhibiting agent is enhanced by lessening the base pair strength between the antisense strand 5′ end (AS 5′) and the sense strand 3′ end (S 3′) of the f-circRNA inhibiting agent relative to the bond strength or base pair strength between the antisense strand 3′ end (AS 3′) and the sense strand 5′ end (S ′5) of said f-circRNA inhibiting agent.

In one embodiment, the asymmetry of an f-circRNA inhibiting agent of the invention may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of an f-circRNA inhibiting agent of the invention may be enhanced such that there is at least one mismatched base pair between the 5′ end of the first or antisense strand and the 3′ end of the sense strand portion. Preferably, the mismatched base pair is selected from the group consisting of G:A, C:A, C:U, G:G, A:A, C:C and U:U. In another embodiment, the asymmetry of an f-circRNA inhibiting agent of the invention may be enhanced such that there is at least one wobble base pair, e.g., G:U, between the 5′ end of the first or antisense strand and the 3′ end of the sense strand portion. In another embodiment, the asymmetry of an f-circRNA inhibiting agent of the invention may be enhanced such that there is at least one base pair comprising a rare nucleotide, e.g., inosine (I). Preferably, the base pair is selected from the group consisting of an I:A, I:U and I:C. In yet another embodiment, the asymmetry of an f-circRNA inhibiting agent of the invention may be enhanced such that there is at least one base pair comprising a modified nucleotide. In preferred embodiments, the modified nucleotide comprises a nucleobase selected from the group consisting of 2-aminopurine and 2,6-diaminopurine.

3) F-circRNA Inhibiting Agents with Enhanced Stability

The f-circRNA inhibiting agents of the present invention can be modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a preferred aspect, the invention features f-circRNA inhibiting agents that include first and second strands wherein the second strand and/or first strand is modified by the substitution of internal nucleotides with modified nucleotides, such that in vivo stability is enhanced as compared to a corresponding unmodified f-circRNA inhibiting agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In a preferred embodiment of the present invention, the f-circRNA inhibiting agents may contain at least one modified nucleotide analogue. The one or more nucleotide analogues may be located at positions where the target-specific silencing activity, e.g., the RNAi mediating activity is not substantially effected, e.g., in a region at the 5′-end and/or the 3′-end of the siRNA molecule. Particularly, the ends may be stabilized by incorporating modified nucleotide analogues.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In particular embodiments, the modifications are 2′-fluoro, 2′-amino and/or 2′-thio modifications. Particularly preferred modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a particular embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents moities of the instant invention. Additional modified residues include, deoxy-abasic, inosine, N3-methyl-uridine, N6,N6-dimethyl-adenosine, pseudouridine, purine ribonucleoside and ribavirin. In a particularly preferred embodiment, the 2′ moiety is a methyl group such that the linking moiety is a 2′-O-methyl oligonucleotide.

In an exemplary embodiment, the f-circRNA inhibiting agent of the invention comprises Locked Nucleic Acids (LNAs). LNAs comprise sugar-modified nucleotides that resist nuclease activities (are highly stable) and possess single nucleotide discrimination for f-circRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-0,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the f-circRNA inhibiting agent of the invention comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone which is highly resistant to nuclease digestion and imparts improved binding specificity to the molecule (Nielsen, et al., Science, (2001), 254: 1497-1500).

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; 0- and N-alkylated nucleotides, e.g., N6-methyl adenosine are suitable. It should be noted that the above modifications may be combined.

In other embodiments, cross-linking can be employed to alter the pharmacokinetics of the f-circRNA inhibiting agent, for example, to increase half-life in the body. Thus, the invention includes f-circRNA inhibiting agents having two complementary strands of nucleic acid, wherein the two strands are crosslinked. The invention also includes f-circRNA inhibiting agents which are conjugated or unconjugated (e.g., at its 3′ terminus) to another moiety (e.g. a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Other exemplary modifications include: (a) 2′ modification, e.g., provision of a 2′ OMe moiety on a U in a sense or antisense strand, but especially on a sense strand, and/or a 2′ F moiety on a U in a sense or antisense strand, but especially on a sense strand, and/or a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context) and/or a 2′ F moiety; (b) modification of the backbone, e.g., with the replacement of an 0 with an S, in the phosphate backbone, e.g., the provision of a phosphorothioate modification, on the U or the A or both, especially on an antisense strand; e.g., with the replacement of a P with an S; (c) replacement of the U with a C5 amino linker; (d) replacement of an A with a G (sequence changes are preferred to be located on the sense strand and not the antisense strand); and (d) modification at the 2′, 6′, 7′, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ OMe moiety and modification of the backbone, e.g., with the replacement of a P with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

4) Modifications to Enhance Cellular Uptake

In other embodiments, f-circRNA inhibiting agents may be modified with chemical moieties, for example, to enhance cellular uptake by target cells (e.g., neuronal cells). Thus, the invention includes f-circRNA inhibiting agents which are conjugated or unconjugated (e.g., at its 3′ terminus) to another moiety (e.g. a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a particular embodiment, an f-circRNA inhibiting agent of invention is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the 3′ end of the sense strand. In certain embodiments, the lipophilic moiety is selected from the group consisting of cholesterol, vitamin E, vitamin K, vitamin A, folic acid, or a cationic dye (e.g., Cy3). In an exemplary embodiment, the lipophilic moiety is a cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

5) Tethered Ligands

Other entities can be tethered to an f-circRNA inhibiting agent of the invention. For example, a ligand tethered to an f-circRNA inhibiting agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. These are preferably located in an internal region, such as in a bulge of an f-circRNA inhibiting agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target f-circRNA inhibition by cleavage of the target nucleic acid. The cleaving group can be, for example, a bleomycin (e.g., bleomycin-A5, bleomycin-A2, or bleomycin-B2), pyrene, phenanthroline (e.g., 0-phenanthroline), a polyamine, a tripeptide (e.g., lys-tyr-lys tripeptide), or metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to an f-circRNA inhibiting agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an f-circRNA inhibiting agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine has an increased affinity for the HIV Rev-response element (RRE). In some embodiments the guanidine analog (the guanidinoglycoside) of an aminoglycoside ligand is tethered to an f-circRNA inhibiting agent. In a guanidinoglycoside, the amine group on the amino acid is exchanged for a guanidine group. Attachment of a guanidine analog can enhance cell permeability of an f-circRNA inhibiting agent. A tethered ligand can be a poly-arginine peptide, peptoid or peptidomimetic, which can enhance the cellular uptake of an oligonucleotide agent.

Exemplary ligands are coupled, preferably covalently, either directly or indirectly via an intervening tether, to a ligand-conjugated carrier. In exemplary embodiments, the ligand is attached to the carrier via an intervening tether. In exemplary embodiments, a ligand alters the distribution, targeting or lifetime of an f-circRNA inhibiting agent into which it is incorporated. In exemplary embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified f-circRNA inhibiting agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a placental cell, a kidney cell and/or a liver cell. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g, cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁₁, C₁₂, C₁₃, C₁₄, C₁₅, C₁₆, C₁₇, C₁₈, C₁₉, or C₂₀ fatty acids) and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the f-circRNA inhibiting agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the f-circRNA inhibiting agent into the cell by activating an inflammatory response, for example. Exemplary ligands that would have such an effect include tumor necrosis factor alpha (TNFα), interleukin-1 beta, or gamma interferon. In one aspect, the ligand is a lipid or lipid-based molecule. Such a lipid or lipid-based molecule preferably binds a serum protein, e.g., human serum albumin (HSA). An HSA binding ligand allows for distribution of the conjugate to a target tissue. For example, the target tissue can be the placenta, the kidneys or the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid based ligand can be used to modulate, e.g., control the binding of the conjugate to a target tissue. For example, a lipid or lipid-based ligand that binds to HSA more strongly will be less likely to be targeted to the placenta, liver and/or kidney and therefore less likely to be cleared from the body. A lipid or lipid-based ligand that binds to HSA less strongly can be used to target the conjugate to the placenta, liver and/or kidney. Other moieties that target to placental, liver and/or kidney cells can also be used in place of or in addition to the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the f-circRNA inhibiting agent, such as by enhancing cellular recognition and absorption. The peptide or peptidomimetic moiety can be about 5-50 amino acids long, e.g., about 5, 10, 15, 20, 25, 30, 35, 40, 45 or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an f-circRNA inhibiting agent via an incorporated monomer unit is a cell targeting peptide such as an arginine-glycine-aspartic acid (RGD)-peptide, or RGD mimic A peptide moiety can range in length from about 5 amino acids to about 40 amino acids. The peptide moieties can have a structural modification, such as to increase stability or direct conformational properties. Any of the structural modifications described below can be utilized.

V. Methods of Introducing Nucleic Acids, Vectors and Host Cells

F-circRNA inhibiting agents of the invention may be directly introduced into the cell (e.g., a neural cell) (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

F-circRNA inhibiting agents, e.g., RNA silencing agents, of the invention can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The nucleic acid may be introduced along with other components that perform one or more of the following activities: enhance nucleic acid uptake by the cell or other-wise increase inhibition of the target gene.

Physical methods of introducing nucleic acids include injection of a solution containing an f-circRNA inhibiting agent, bombardment by particles covered by the f-circRNA inhibiting agent, soaking the cell or organism in a solution of the f-circRNA inhibiting agent, or electroporation of cell membranes in the presence of the f-circRNA inhibiting agent. A viral construct packaged into a viral particle would accomplish both efficient introduction of an expression construct into the cell and transcription of f-circRNA inhibiting agent encoded by the expression construct. Other methods known in the art for introducing nucleic acids to cells may be used, such as lipid-mediated carrier transport, chemical-mediated transport, such as calcium phosphate, and the like. Thus the f-circRNA inhibiting agent may be introduced along with components that perform one or more of the following activities: enhance f-circRNA inhibiting agent uptake by the cell, inhibit annealing of single strands, stabilize the single strands, or otherwise increase inhibition of the target gene.

An f-circRNA inhibiting agent may be directly introduced into the cell (i.e., intracellularly); or introduced extracellularly into a cavity, interstitial space, into the circulation of an organism, introduced orally, or may be introduced by bathing a cell or organism in a solution containing the f-circRNA inhibiting agent. Vascular or extravascular circulation, the blood or lymph system, and the cerebrospinal fluid are sites where the f-circRNA inhibiting agent may be introduced.

The cell having the target f-circRNA may be from the germ line or somatic, totipotent or pluripotent, dividing or non-dividing, parenchyma or epithelium, immortalized or transformed, or the like. The cell may be a stem cell or a differentiated cell. Cell types that are differentiated include, but are not limited to, adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, cells of the endocrine or exocrine glands and the like.

Depending on the particular target f-circRNA and the dose of double stranded RNA material delivered, this process may provide partial or complete elimination of the presence of an f-circRNA. A reduction or loss of f-circRNA in at least 50%, 60%, 70%, 80%, 90%, 95% or 99% or more of targeted cells is exemplary. Inhibition of f-circRNA refers to the absence (or observable decrease) in f-circRNA levels or one or more f-circRNA activities. Specificity refers to the ability to inhibit the f-circRNA without manifesting effects on other RNA species (e.g., mRNA, circRNA and the like) of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, Enzyme Linked ImmunoSorbent Assay (ELISA), Western blotting, RadioImmunoAssay (RIA), other immunoassays, and Fluorescence Activated Cell Sorting (FACS).

The F-circRNA inhibiting agent, e.g., RNAi agent or DNA binding agent, may be introduced in an amount which allows delivery of at least one copy per cell. Higher doses (e.g., at least 5, 10, 100, 500 or 1000 copies per cell) of material may yield more effective inhibition; lower doses may also be useful for specific applications.

In an exemplary aspect, the efficacy of an F-circRNA inhibiting agent, e.g., an RNAi agent or DNA binding agent, of the invention is tested for its ability to specifically degrade f-circRNA in cells. Readily transfectable cells suitable for cell-based validation assays include, for example, trophoblast cells, HeLa cells or COS cells. Cells are transfected with a vector expressing f-circRNA or a complement thereof as described further herein. Standard siRNA, modified siRNA or vectors able to produce siRNA from U-looped mRNA are co-transfected. Selective reduction in target f-circRNA is measured. Reduction of target f-circRNA can be compared to levels of f-circRNA in the absence of an F-circRNA inhibiting agent or in the presence of an inhibiting agent that does not target f-circRNA. Exogenously-introduced f-circRNA can be assayed for comparison purposes.

6) Recombinant Adeno-Associated Viruses and Vectors

In certain exemplary embodiments, recombinant adeno-associated viruses (rAAVs) and their associated vectors can be used to deliver one or more F-circRNA inhibiting agent (e.g., siRNAs and/or shRNAs) into cells. AAV is able to infect many different cell types, although the infection efficiency varies based upon serotype, which is determined by the sequence of the capsid protein. Several native AAV serotypes have been identified, with serotypes 1-9 being the most commonly used for recombinant AAV. AAV-2 is the most well-studied and published serotype. The AAV-DJ system includes serotypes AAV-DJ and AAV-DJ/8. These serotypes were created through DNA shuffling of multiple AAV serotypes to produce AAV with hybrid capsids that have improved transduction efficiencies in vitro (AAV-DJ) and in vivo (AAV-DJ/8) in a variety of cells and tissues.

In particular embodiments, widespread central nervous system (CNS) delivery can be achieved by intravascular delivery of recombinant adeno-associated virus 7 (rAAV7), RAAV9 and rAAV10, or other suitable rAAVs (Zhang et al. (2011) Mol. Ther. 19(8):1440-8. doi: 10.1038/mt.2011.98. Epub 2011 May 24). rAAVs and their associated vectors are well-known in the art and are described in US Patent Applications 2014/0296486, 2010/0186103, 2008/0269149, 2006/0078542 and 2005/0220766, each of which is incorporated herein by reference in its entirety for all purposes.

rAAVs may be delivered to a subject in compositions according to any appropriate methods known in the art. An rAAV can be suspended in a physiologically compatible carrier (i.e., in a composition), and may be administered to a subject, i.e., a host animal, such as a human, mouse, rat, cat, dog, sheep, rabbit, horse, cow, goat, pig, guinea pig, hamster, chicken, turkey, a non-human primate (e.g., baboon) or the like. In certain embodiments, a host animal is a non-human host animal.

Delivery of one or more rAAVs to a mammalian subject may be performed, for example, by intramuscular injection or by administration into the bloodstream of the mammalian subject. Administration into the bloodstream may be by injection into a vein, an artery, or any other vascular conduit. In certain embodiments, one or more rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer virions into the vasculature of an isolated limb to potentially enhance transduction into muscle cells or tissue. Moreover, in certain instances, it may be desirable to deliver virions to the placenta, liver and/or kidneys of a subject. Recombinant AAVs may be delivered directly to the placenta, liver and/or kidney with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

The compositions of the invention may comprise an rAAV alone, or in combination with one or more other viruses (e.g., a second rAAV encoding having one or more different transgenes). In certain embodiments, a composition comprises 1, 2, 3, 4, 5, 6, 7, 8, 9, 10 or more different rAAVs each having one or more different transgenes.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of one or more rAAVs is generally in the range of from about 1 ml to about 100 ml of solution containing from about 10⁹ to 10¹⁶ genome copies. In some cases, a dosage between about 10¹¹ to 10¹² rAAV genome copies is appropriate. In certain embodiments, 10¹² rAAV genome copies is effective to target heart, liver, and pancreas tissues. In some cases, stable transgenic animals are produced by multiple doses of an rAAV.

In some embodiments, rAAV compositions are formulated to reduce aggregation of AAV particles in the composition, particularly where high rAAV concentrations are present (e.g., about 10¹³ genome copies/mL or more). Methods for reducing aggregation of rAAVs are well known in the art and, include, for example, addition of surfactants, pH adjustment, salt concentration adjustment, etc. (See, e.g., Wright et al. (2005) Molecular Therapy 12:171-178, the contents of which are incorporated herein by reference.)

“Recombinant AAV (rAAV) vectors” comprise, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., siRNA) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are usually about 145 basepairs in length. In certain embodiments, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including mammalian AAV types described further herein.

VI. Kits

In certain aspects, the invention provides kits that include a suitable container containing a pharmaceutical formulation of an f-circRNA inhibiting agent, e.g., an f-circRNA inhibiting agent, e.g., a double-stranded RNA silencing agent, or sRNA agent, (e.g., a precursor, e.g., a larger RNA silencing agent which can be processed into a sRNA agent, or a DNA which encodes an RNA silencing agent, e.g., a double-stranded RNA silencing agent, or sRNA agent, or precursor thereof). In certain aspects, the invention provides kits that include a suitable container containing a nucleic acid sequence encoding an f-circRNA. In certain embodiments the individual components of the pharmaceutical formulation may be provided in one container. Alternatively, it may be desirable to provide the components of the pharmaceutical formulation separately in two or more containers, e.g., one container for an f-circRNA inhibiting agent preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

In certain aspects, the invention provides kits that include a suitable container containing a nucleic acid sequence (e.g., an expression vector) encoding an f-circRNA or a complement thereof and optional reagents for use with the vector. In certain embodiments, the individual components of the kit may be provided in one container. Alternatively, it may be desirable to provide the components of the kit separately in two or more containers, e.g., one container for an expression vector, and at least another for a buffer and/or other reagents. The kit may be packaged in a number of different configurations such as one or more containers in a single box. The different components can be combined, e.g., according to instructions provided with the kit. The components can be combined according to a method described herein, e.g., to express an f-circRNA or complement thereof from an expression vector. The kit can also include a delivery vehicle (e.g., one or more transfection reagents).

### EXAMPLES

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### Example 1: f-circRNAs are a Product of Aberrant Chromosomal Translocations in Cancer Cells

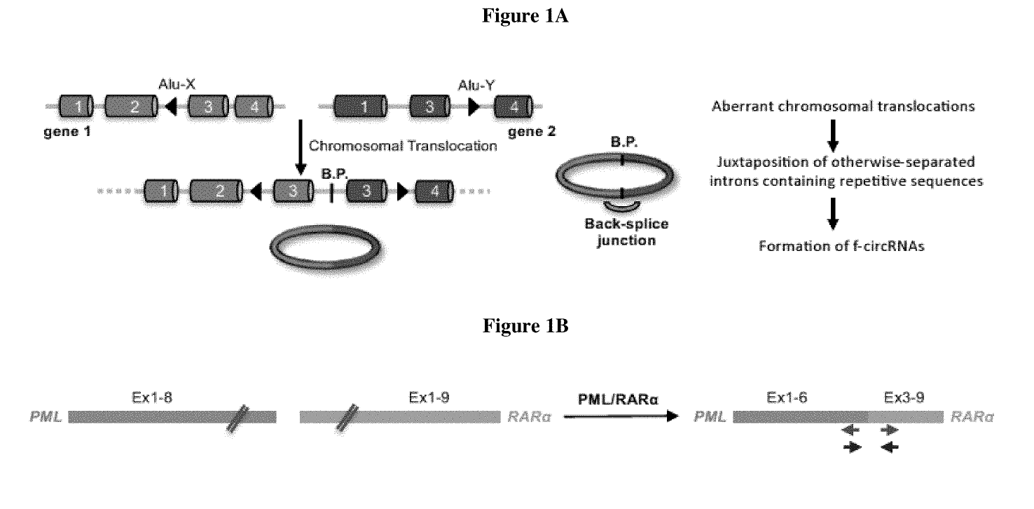

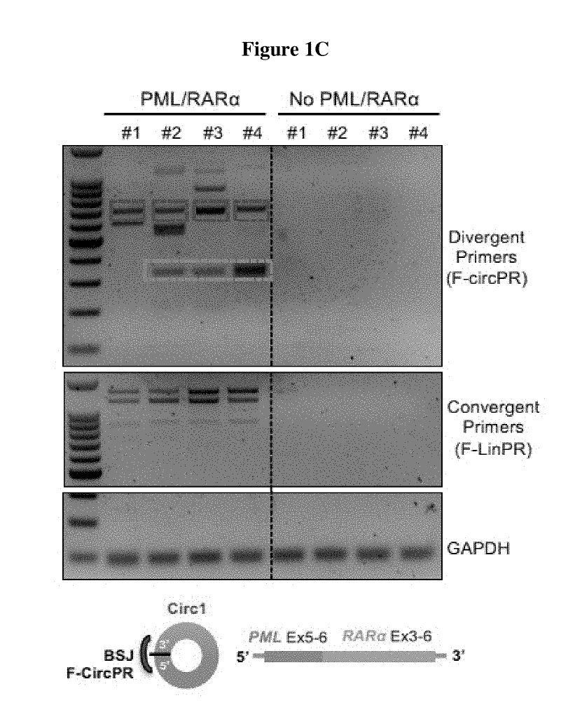

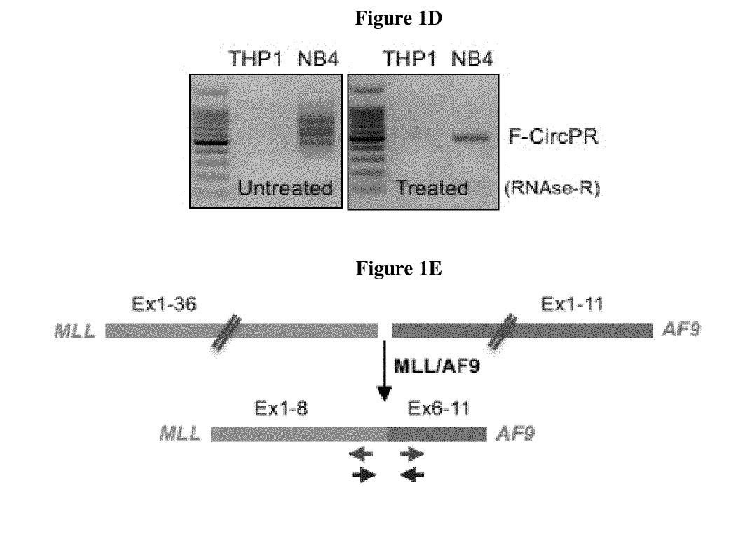

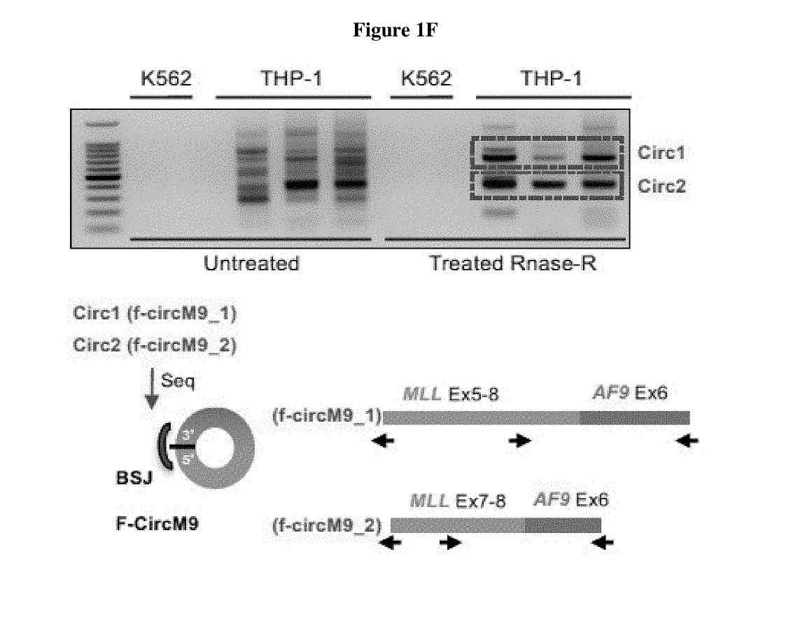

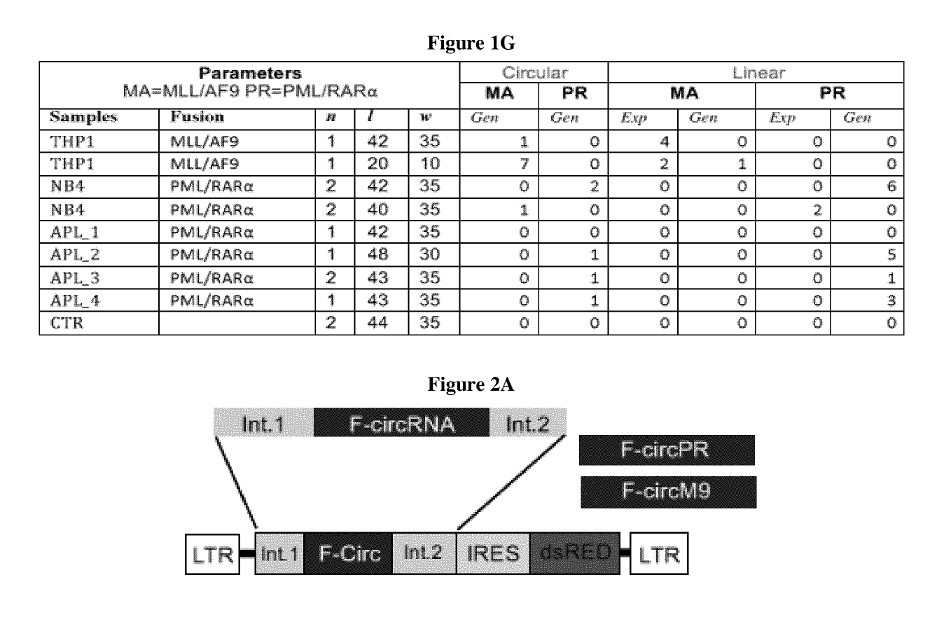

Multiple tumor types are characterized by the presence of aberrant chromosomal translocations and chromosomal rearrangements (Rabbitts, 1994; Bunting and Nussenzweig, 2013). These rearrangements result first in the juxtaposition of two otherwise-separated genes, then in the transcription of a fused mature mRNA and finally in the expression of specific fusion oncogenic proteins (Rabbitts, 1994). Because genes are incorrectly reallocated, it was hypothesized that distant, unrelated intronic sequences of the translocated genes may also be juxtaposed. Complementary repetitive intronic sequences (e.g. Alu-sequences) could be brought in at a close enough proximity to favor new events of back-splicing during the process or RNA maturation, which would result in the generation of aberrant circRNAs (Barrett et al., 2015; Jeck et al., 2013; Liang and Wilusz, 2014; Zhang et al., 2014). It was thus hypothesized that the juxtaposition of complementary sequences in introns up- and down-stream of the breakpoint region of the translocation could allow the formation of new circRNAs (f-circRNAs) made by the fusion of the two translocated genes (FIG. 1A). In order to investigate this possibility, primers diverging from the breakpoint of the translocation were at first used and then the presence of f-circRNAs through PCR assays was searched for.

Several types of leukemia are known to carry distinctive chromosomal translocations, among them, PML-RARα is the most recurrent translocation in patients with acute promyelocytic leukemia (APL) (Dos Santos et al., 2013). The main break point cluster region (BCR) of the PML gene is located in intron 6, while RARα, the partner gene of PML, shows BCR in intron 2 (FIG. 1B) (Dekking et al., 2012). Using primary samples from patients that harbor this translocation, whether PML/RARα could generate f-circRNAs (f-circPR for brevity) was investigated. Total RNA was extracted from bone marrow- (BM) derived leukemic cells of APL patients, or human primary leukemic cells as control, and subjected to treatment with RNase R (Jeck and Sharpless, 2014). Divergent primers were next used to amplify possible f-circPRs (FIG. 1B and FIG. 7A). All of the analyzed patients that carried the PML/RARα translocation (confirmed by the use of convergent primers spanning the break-point, FIG. 1B-C) displayed the expression of one or more f-circPRs (FIG. 1C). By sequencing the PCR products, is was ascertained that all of the patients expressed an isoform of f-circPR with the back-splice junction between the 5′-head of PML exon 5 and the 3′-tail of RARα exon 6 (FIG. 7). However, 3 out of 4 patients showed expression of an additional, alternative f-circPR, whose back-splice junction occurred between exon 4 of PML and exon 4 of RARα (FIG. 1C and FIG. 7B). In order to further validate these results, the presence of f-circRNA in the APL-derived leukemic cell line NB4 was also analyzed (Lanotte et al., 1991). Only one f-circRNA was detected in the NB4 cells, whose back-splice junction was sequenced between the 5′-head of exon 5 of PML and the 3′-tail of exon 6 of RARα (FIG. 1D and FIG. 7C). It was concluded that as a result of a paradigmatic chromosomal translocation such as the t(15;17) of APL, the PML/RARα fusion gene can give raise to one or more f-circRNAs.

The investigation of f-circRNAs was next extended to other chromosomal translocations. The MLL gene has multiple fusion partners in acute myeloid leukemia (Krivtsov and Armstrong, 2007), and among them, a recurrent fusion partner is AF9 (MLLT3). Whether the MLL/AF9 aberrant translocation could also generate f-circRNAs was thus investigated. To this end, THP1 cells were used in which MLL was broken after exon 8 and was joined to AF9, which causes a loss of its first 5 exons (Tsuchiya et al., 1980), FIG. 1E and FIG. 7D) Amplification with primers diverging from the breakpoint followed by Sanger sequencing revealed that the MLL/AF9 translocation bore 2 distinct f-circRNAs (f-circM9_1 and f-circM9_2 for brevity, FIG. 1F and FIG. 7E). f-circM9_2 displayed its back-splice junction between the 5′-head of MLL exon 7 and the 3′-tail of AF9 exon 6, while f-circM9_1 displayed its back-splice junction between the 5′-head of MLL exon 5 and the 3′-tail of AF9 exon 6 (FIG. 1F and FIG. 7E). Thus, multiple f-circRNAs could be generated from PML-RARα and MLL/AF9 fusion genes.

Finally, whether the formation of f-circRNAs is an exclusive feature of leukemic cells, or whether it is also common to tumors of different histological origins, was investigated. To this end, solid tumors were analyzed, and whether the EWSR1-FLI1 translocation, associated with Ewing Sarcoma (Anderson et al., 2012), and the EML4/ALK1 translocation, associated with lung cancer (Martelli et al., 2009), could also originate f-circRNAs was investigated. SK-NEP Ewing sarcoma cells (FIG. 8A-B) (Smith et al., 2008), and H3122 lung cancer cells were therefore used for this analysis. Sanger sequencing revealed that sarcoma cells displayed expression of f-circRNAs composed by EWSR1 and its partner FLI1 (f-circEF1), with the back-splice junction encompassing the 5′-head of EWSR1 exon 7, and the 3′-tail of exon 10 of FLI1 (FIG. 8A-B). Similarly, lung cancer cell lines harboring the EML4/ALK1 translocation (FIG. 8C) showed the expression of a f-circRNA composed by EML4 and ALK1 (f-circEA1), with the back-splice junction located between the 5′-head of EML4 exon 12 and the 3′-tail of ALK1 exon 26 (FIG. 8D).

An alternative approach for the identification of f-circRNAs was used next. To this end, samples whose expression of f-circRNAs was previously assessed by PCR, were submitted to RiboMinus-enriched RNA-sequencing, and next analyzed the presence of reads spanning either the breakpoint region or the back-splice junction. Two different approaches were used for the analysis of the f-circRNAs: Method I and Method II.

As reported in FIGS. 8E and 8F, Method I consists of a single read mapping step; RNA-seq reads were directly mapped to the linear RNA fusion and f-circRNA fusion reference libraries using bowtie (Langmead et al., 2009). Next, among the mapped reads, reads containing the spliced junction of linear fusions and the back-spliced junction of f-circRNAs were identified. Finally, reads meeting the following three conditions were selected: i) mapping to the f-circRNA or linear RNA fusion reference libraries; ii) containing the spliced or back-spliced junction; and iii) containing at least w bases per read from each gene involved in the fusion. W was defined as the minimum number of bases which produced no false positive reads for the identification of linear fusion reads in both Method I and Method II. A false positive linear fusion read was defined as the identification of a linear fusion read among cases not reported as containing the translocation (i.e., identification of a PML/RARα fusion transcript among a case reported as MLL/AF9).

Method II consists of two read mapping steps. A filtering step was first performed to eliminate all RNA-seq reads that mapped to the UCSC human transcriptome (Guo et al., 2014; Memczak et al., 2013; Salzman et al., 2013). After this filtering step, the remaining unmapped reads were mapped to the linear RNA fusion and f-circRNA fusion reference libraries using Bowtie, and linear fusion RNAs and f-circRNAs were identified using the same three conditions as in Method I.

The results obtained by the application of these pipelines are summarized in FIG. 1G. Notably, for the identification of f-circRNAs, only reads that spanned the back-splice junction of the f-circRNA were counted, and all those reads that mapped shared sequences between the linear transcript and the f-circRNA were excluded. Thus, because of these strict bioinformatics criteria and the fact that the back-splice reads are by definition fewer than the shared reads from flanking sequences, the true prevalence of f-circRNAs in these samples may be higher than that reported in these analyses.

Taken together these experiments and analysis demonstrate that chromosomal rearrangements affect the non-coding RNA dimension, particularly through the generation of a new RNA molecular species, f-circRNAs, within tumor cells.

### Example 2: f-circRNAs are Oncogenic RNAs that Contribute to Cellular Transformation

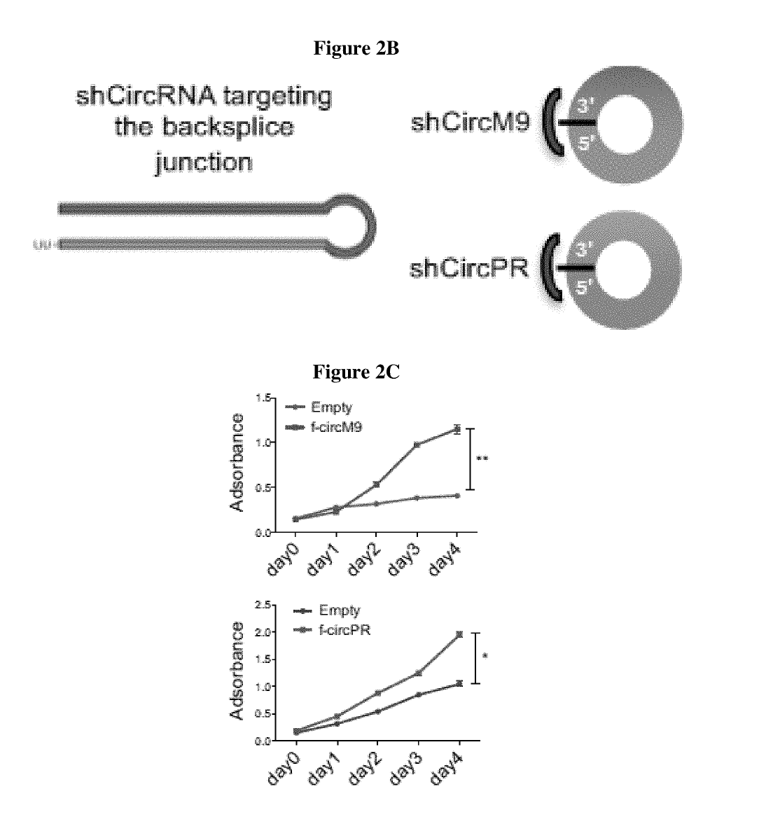

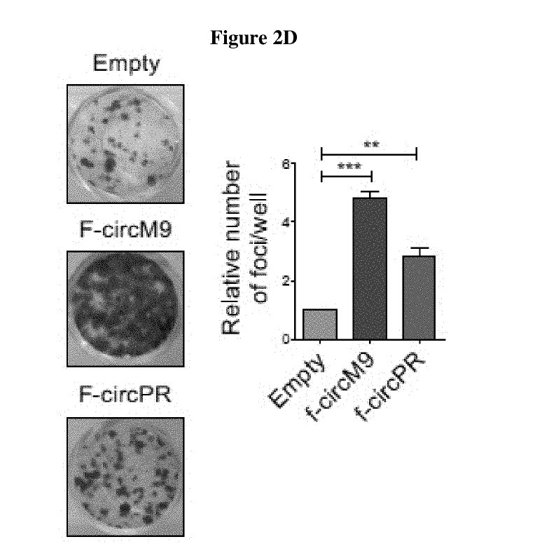

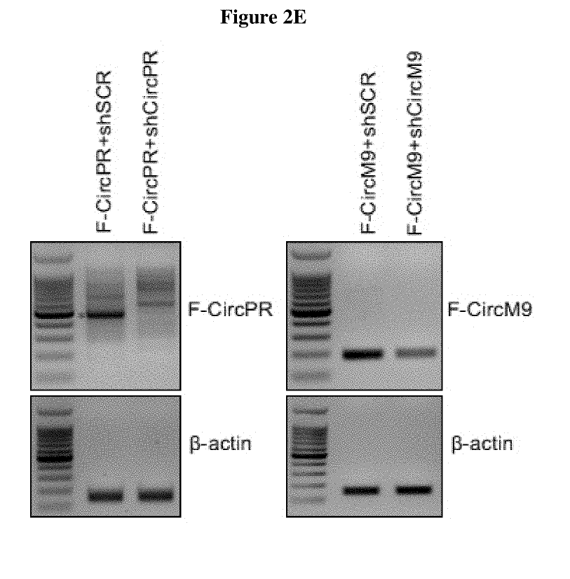

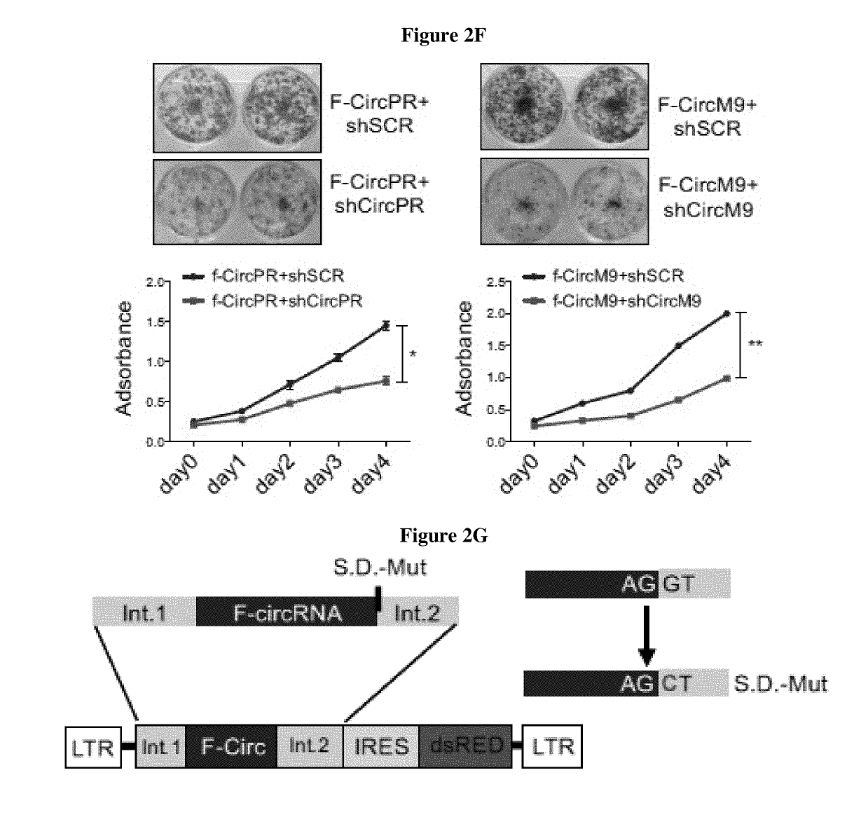

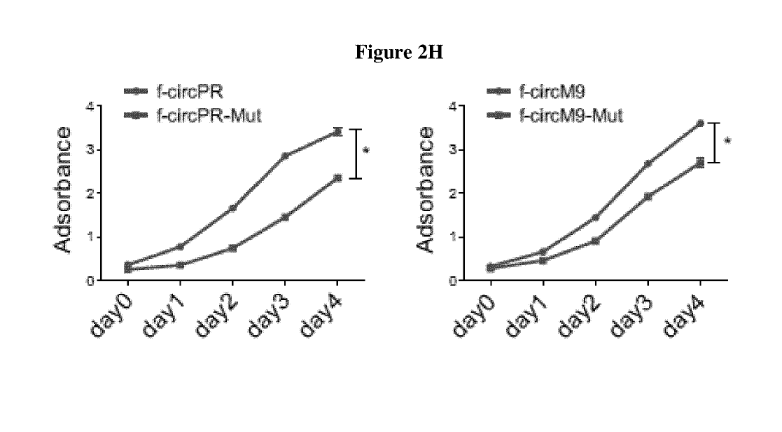

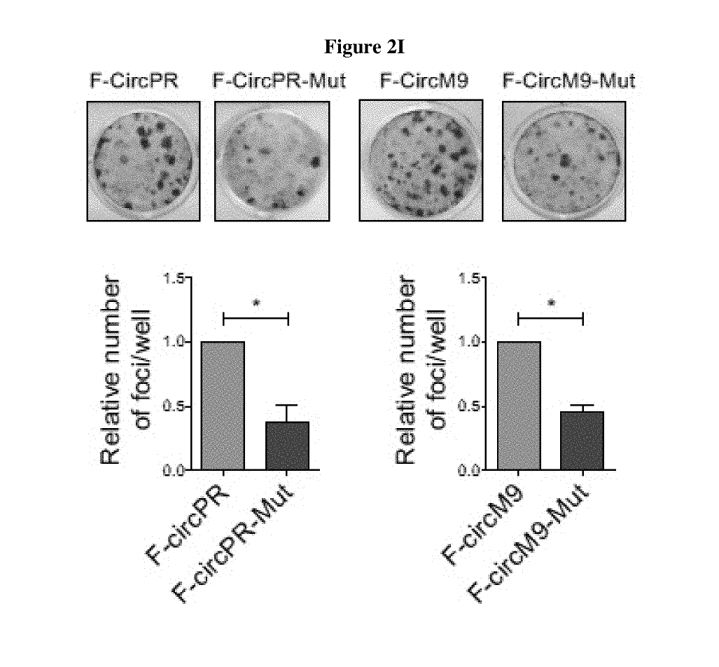

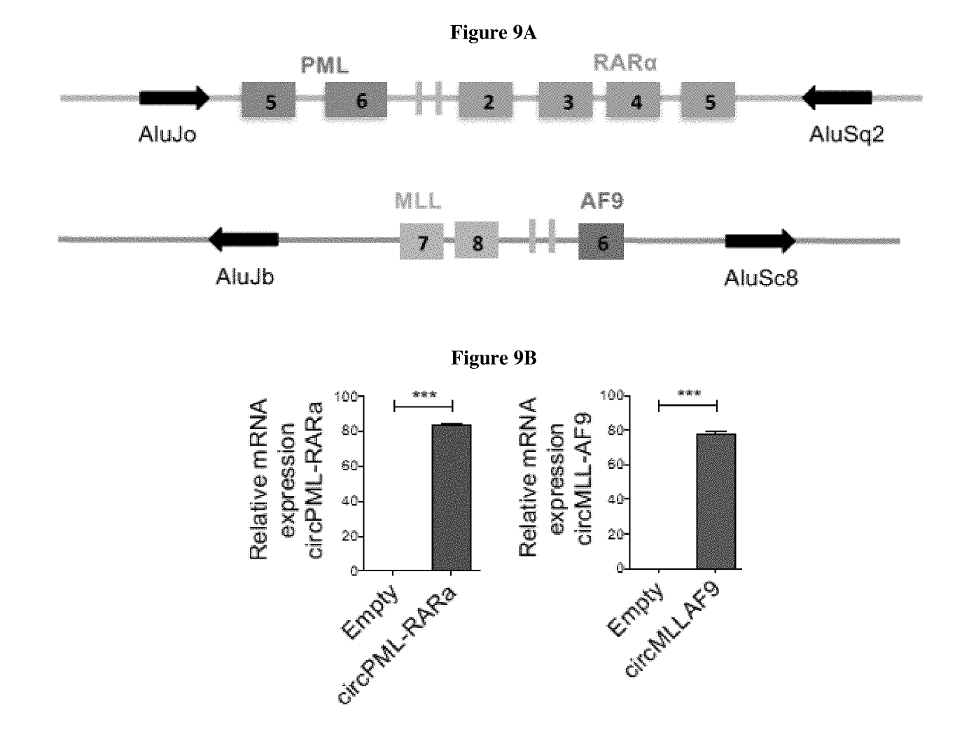

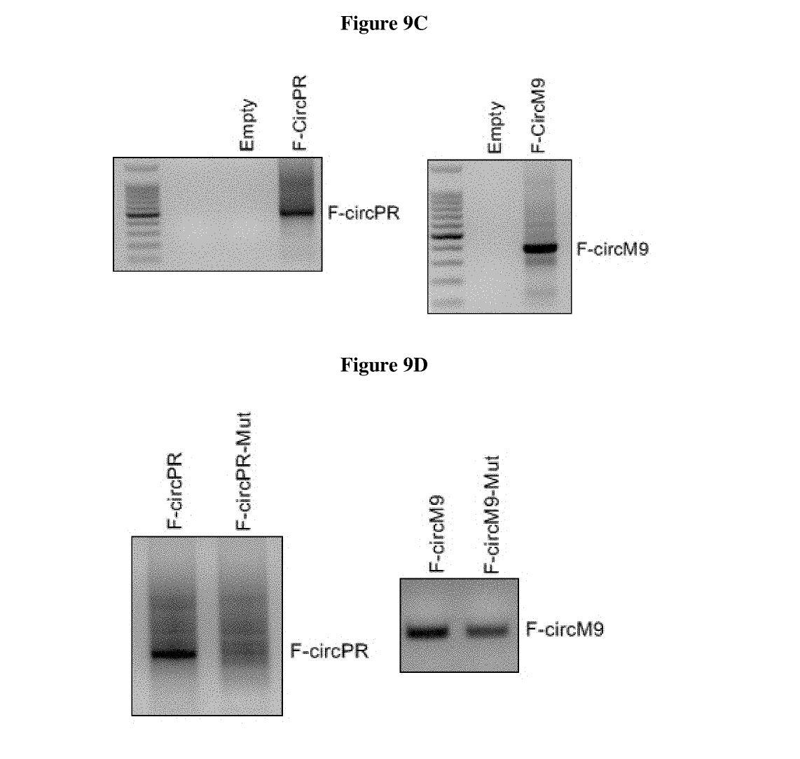

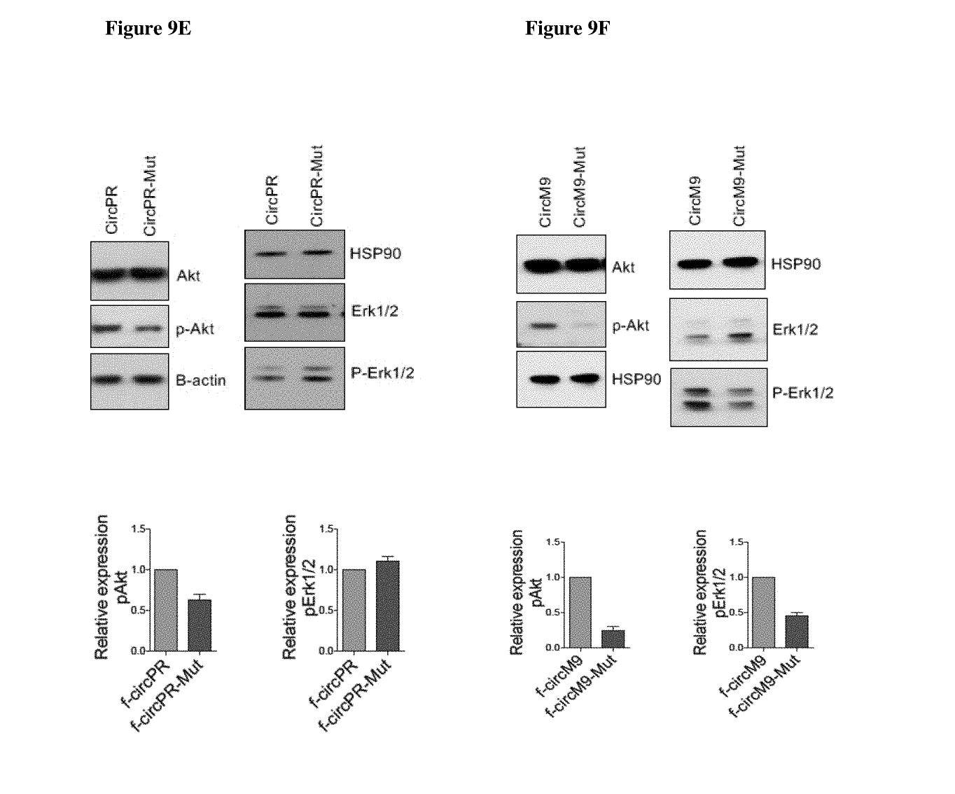

Because translocations are early events in tumorigenesis, and in some circumstances are the primary cause of the tumor onset, whether f-circRNAs themselves could play a functional role in this context was investigated. The specific function of f-circRNAs was studied with the goal of disentangling their possible activity from that of the concurrently present fusion protein in tumor cells. At first, f-circRNAs were expressed in immortalized, but not transformed, mouse embryonic fibroblasts (MEFs; see Example 15) which do not express the translocation and therefore do not express the fusion protein. Circularizing exons were then cloned into expressing vectors, together with up- and down-stream flanking introns in order to favor the spontaneous formation of the f-circRNAs (FIG. 2A and FIG. 9A-C).

The expression of f-circRNAs was assessed by PCR and qRT-PCR analysis of the specific back-splice junction of the f-circRNA. Importantly, expression of f-circRNA was then reverted for functional studies through the use of short hairpin circRNAs (shCircRNAs) targeting their back-splice junction (FIG. 2B). F-circRNA-expressing cells were then compared to control cells (expressing an empty vector) for proliferation rate, loss of contact inhibition and their transformed foci-forming capability. As shown in FIG. 2C, cells expressing f-circPR and f-circM9 both showed a higher proliferative rate when compared to cells expressing the empty vector. Accordingly, their ability to form foci was much higher compared to control cells (FIG. 2D; see Experimental procedures section), suggesting that f-circRNAs are indeed functional and proto-oncogenic.

In order to confirm these results, the expression of f-circRNAs was next silenced by transducing cells with shCircRNAs spanning the f-circRNA back-splice junction (FIG. 2E). Intriguingly, the increased proliferation rate observed in cells expressing f-circRNAs was reverted upon the silencing of the f-circRNAs (FIG. 2F), corroborating the notion that these f-circRNAs (both for f-circPR and f-circM9) exerted proto-oncogenic activity and were involved in the tumorigenic process independent of their linear transcript and their fusion protein counterparts.

In order to have an additional and independent demonstration of the functionality of f-circRNAs, an alternative experimental strategy was next adopted. f-circRNAs-expressing vectors were mutagenized at the splicing donor site (f-circRNA-mut), as documented in Wang et al. (2015) and shown in FIG. 2G. F-circRNA-mut vectors allowed the expression of the linear transcript while impairing the circularization event (FIG. 2G and FIG. 9D) Immortalized MEFs were then transduced with either f-circRNA-mut vectors or regular f-circRNAs expressing vectors, and the cells' proliferation and transformation were then assayed. In line with previous experiments described herein, the expression of f-circRNAs (both for f-circPR and f-circM9) triggered cells to form foci when compared to the expression of f-circRNA-mut vectors (FIG. 2H-I).

Whether the expression of f-circPR and f-circM9 would lead to the activation of common or distinct proto-oncogenic pathways was next tested. Intriguingly, although the expression of f-circPR and f-circM9 showed similar biological outcomes in MEFs, it was found that they triggered differentially the activation of the PI3K and MAPK signal transduction pathways (FIG. 9E-F). This in turn indicated that f-circRNAs may exert their tumorigenic activity through district signaling outputs.

Together these experiments reveal that f-circPR and f-circM9 are biologically active, exerting both pro-proliferative and proto-oncogenic activities.

### Example 3: f-circRNAs Contribute to Leukemia Progression In Vivo

Experiments performed with immortalized MEFs allowed an initial evaluation of the possible involvement of f-circRNAs in cellular transformation. In order to directly test the role of f-circRNAs in tumor cells in vivo, hematopoietic cells were enrolled and the conditions to study the involvement of f-circRNAs during the onset and progression of leukemia were set up. Using the MLL/AF9-AML model, f-circM9 was investigated. At first, the question of whether f-circM9 alone was sufficient to trigger leukemia from hematopoietic stem cells (HSCs) was addressed. To investigate, HSCs were isolated from wild type mice, transduced in vitro with a vector expressing f-circM9 (or an empty vector as control), and either used for in vitro methylcellulose assays or transplanted back into sub-lethally irradiated recipients. Regardless of the expression of f-circM9, HSCs differentiated during the methylcellulose cultures toward exhaustion. Similarly, none of the transplanted mice developed leukemia during following the transplantation of the infected cells in a three-month follow up; only very few donor-derived cells were found in the bone marrow of transplanted mice. These initial experiments suggested that f-circM9 alone is probably not sufficient to trigger tumorigenesis on its own, and additional oncogenic events might be required.

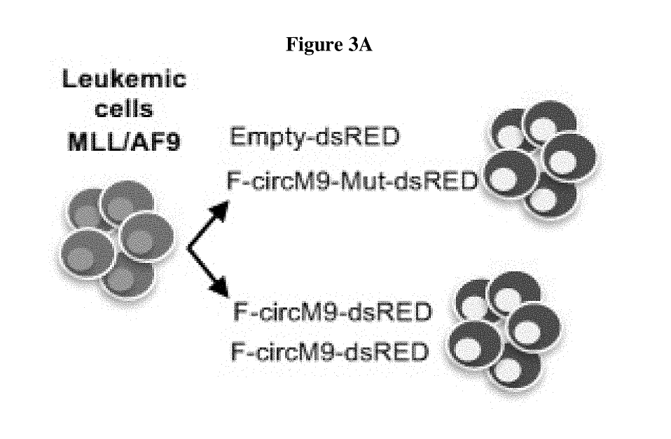

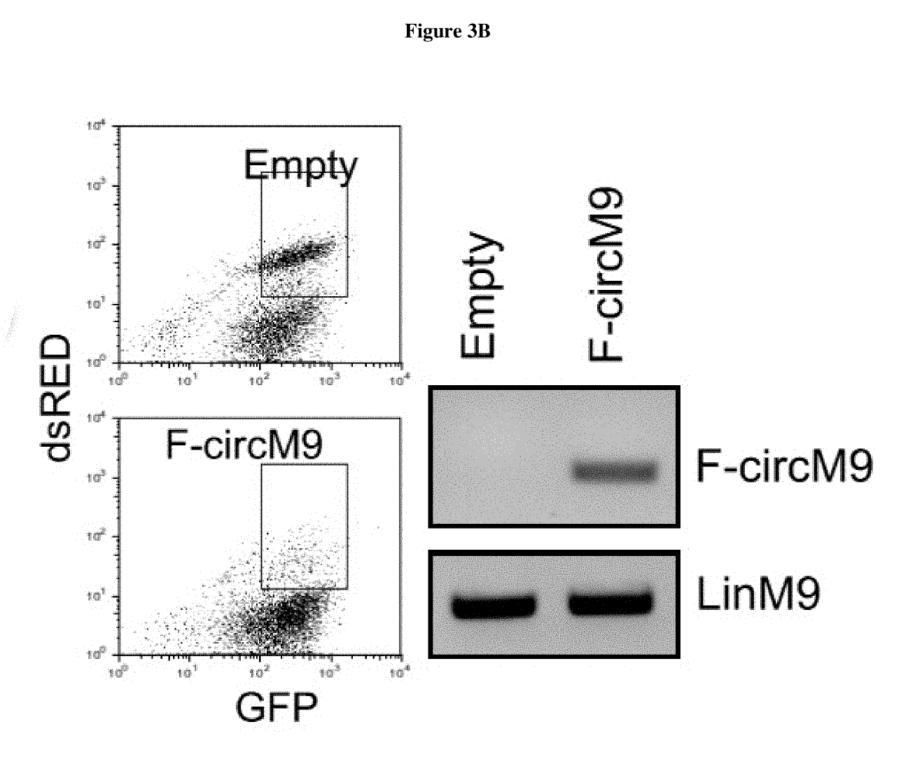

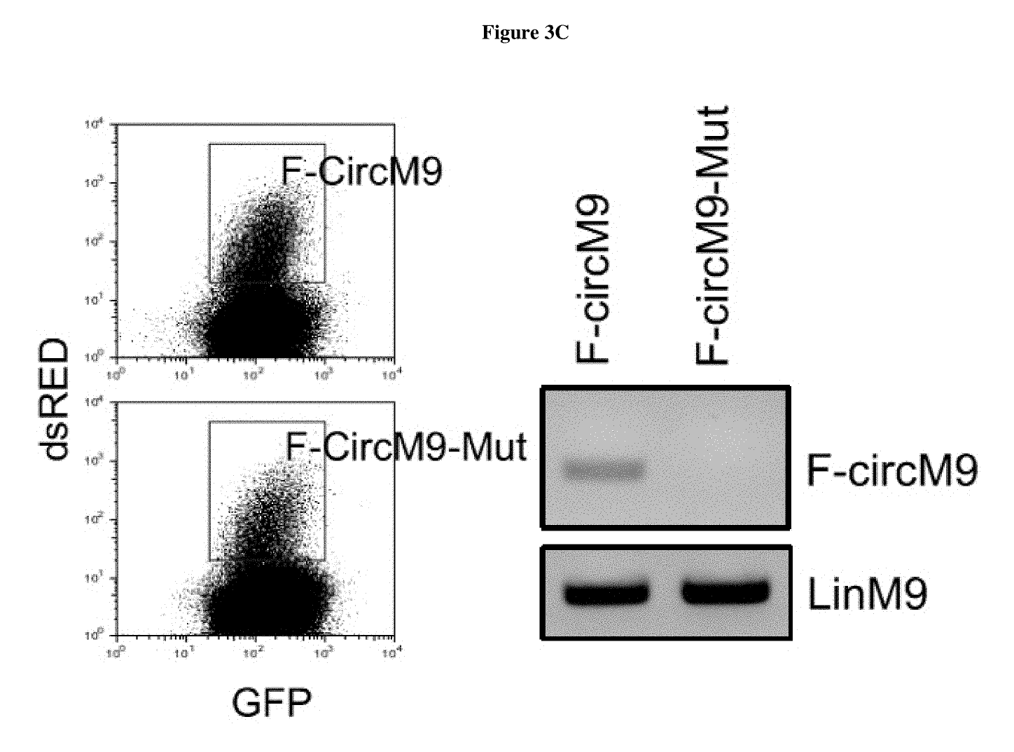

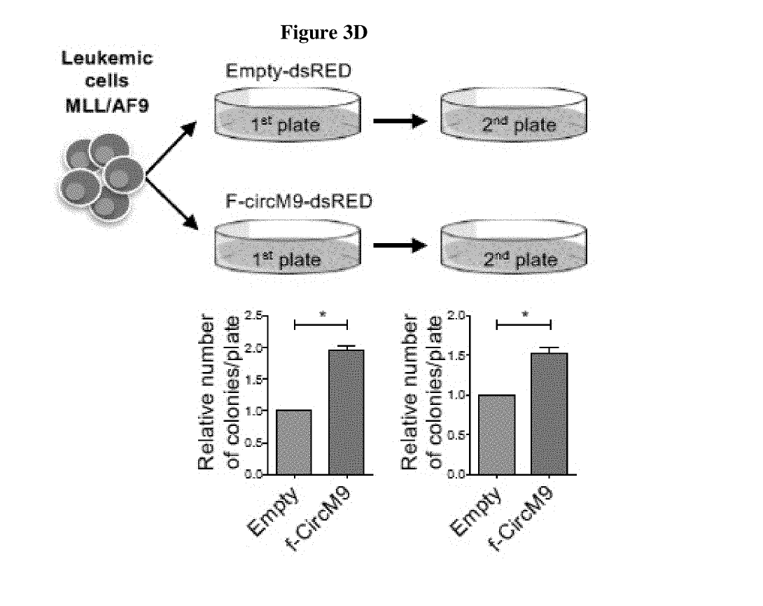

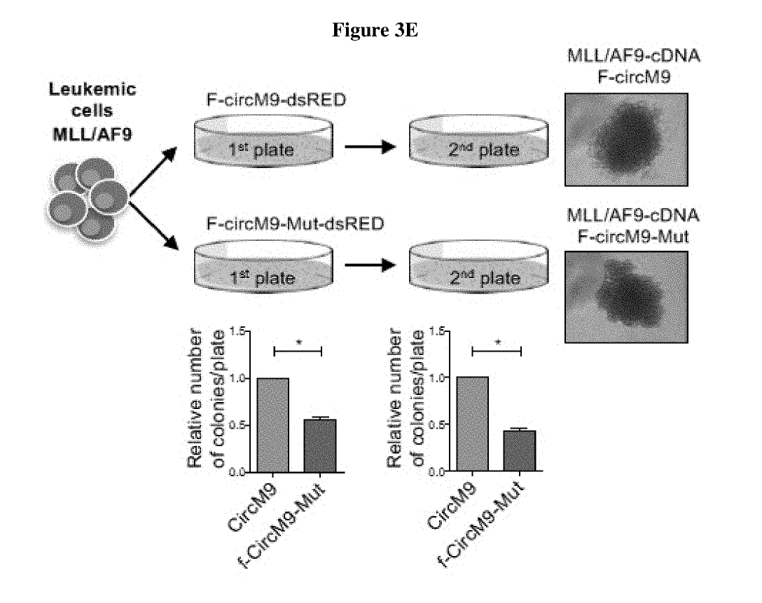

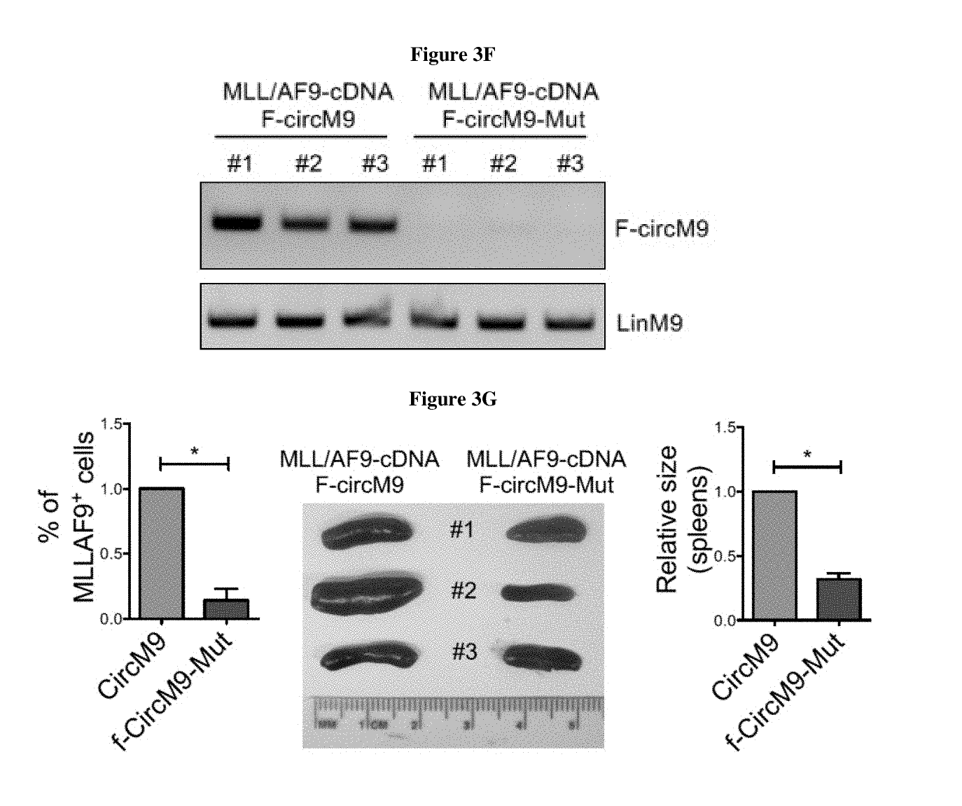

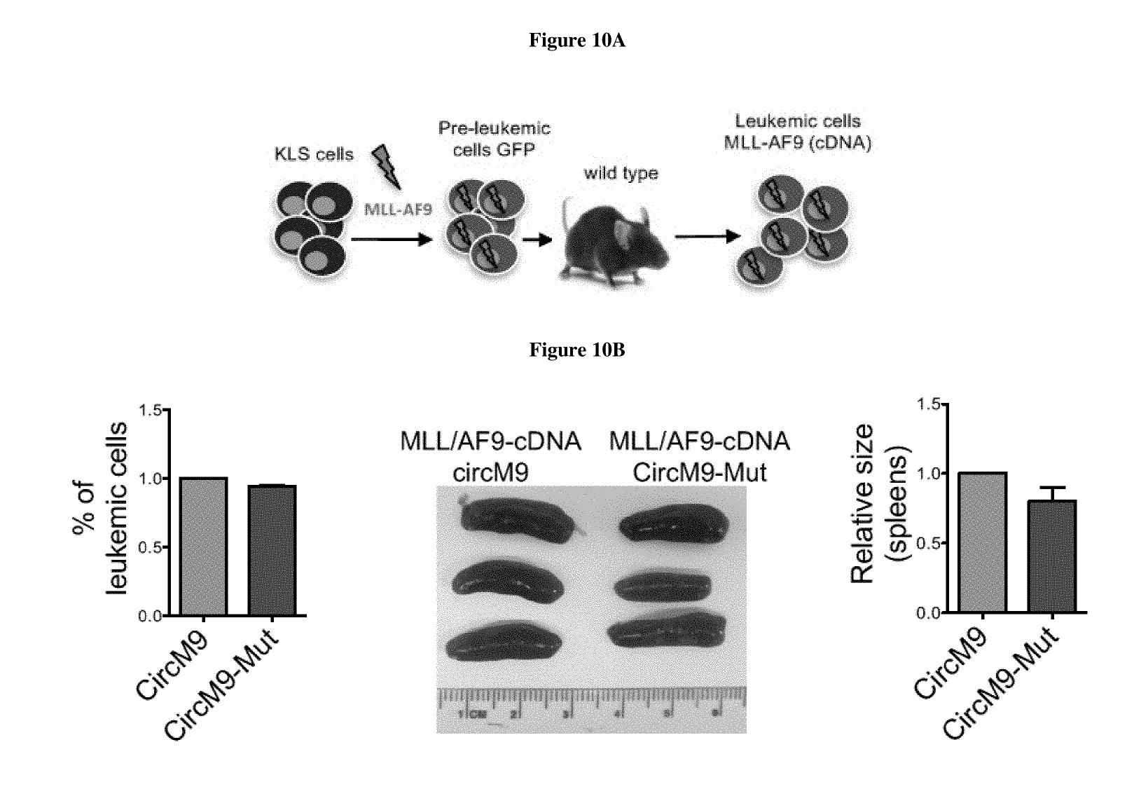

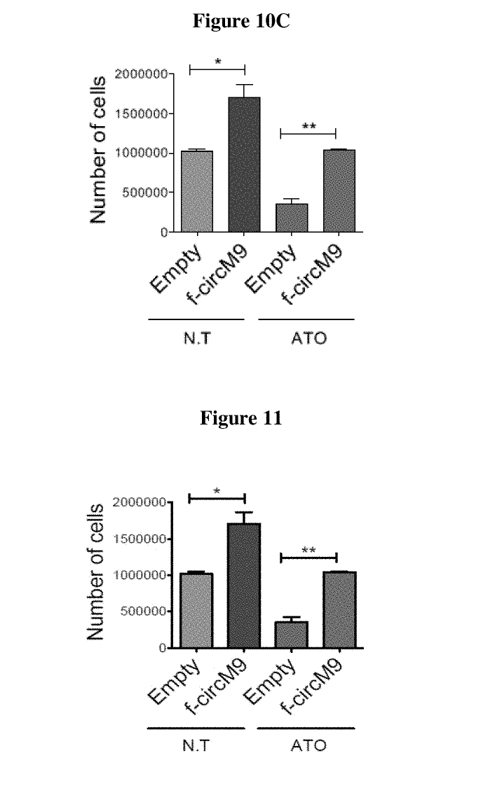

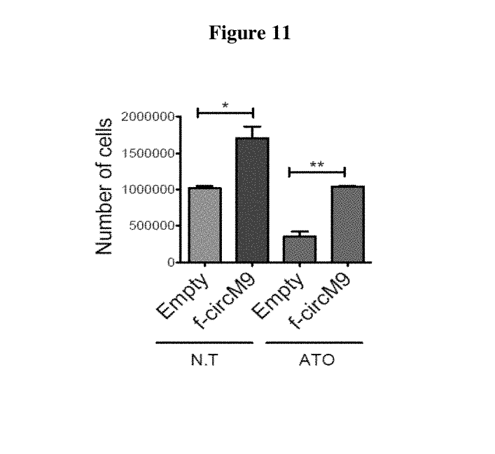

Because at the onset of chromosomal translocations, the formation of f-circRNAs is always coupled with the presence of f-linear RNAs and the encoded oncogenic fusion proteins (which have been already described as fundamental hits for the tumorigenesis process), the relevance of f-circM9 as an oncogene in the presence of its partner, the MLL/AF9 fusion protein, was investigated. HSCs were therefore transduced with a green fluorescent protein- (GFP) labeled retroviral vector which expressed the cDNA of MLL/AF9 as described in FIG. 10A. Infected cells were then transplanted into sub-lethally irradiated mice. The vector expressed the fusion protein and triggered a pre-leukemic phase in the mice. Two months post-transplantation, mice started to develop leukemia, in line with what has been previously reported (Krivtsov and Armstrong, 2007). GFP+ leukemic cells were then sorted from recipient mice and transduced in vitro, either with an empty vector or the f-circM9-expressing vector (FIG. 3A-B). As a further control in an independent experimental setting, leukemic cells were also transduced with the f-circM9-expressing vector or the f-circM9-Mut-expressing vector (FIG. 3A-C). Cells expressing these vectors were re-sorted as dsRED+ cells, and used for in vitro and in vivo experiments. Sorted cells were then plated in vitro in methylcellulose. After five days in culture, proliferation and the capacity to form colonies were evaluated. At the point of colony count, cells were then resuspended and used for a second round of methylcellulose assay. As shown in FIG. 3D, cells expressing f-circM9 together with the MLL/AF9 fusion protein displayed an increased ability to proliferate and form colonies in both serial platings when compared to cells expressing solely the MLL/AF9 fusion protein. Similar results were obtained in the other experimental setting: MLL/AF9-positive cells transduced with the f-circM9 showed proliferative advantage and a higher colony formation capacity when compared to cells transduced with the f-circM9-Mut vector which was deficient for the formation of the f-circM9 (FIG. 3E).

These ex vivo experiments contributed to strengthening the hypothesis that f-circRNAs could contribute to the leukemogenic process. To obtain in vivo evidence of their role in leukemogenesis, in vivo transplantation experiments were next performed. GFP+ leukemic cells (already expressing MLL/AF9 cDNA) were transduced with f-circM9 or f-circM9-Mut vectors, re-sorted as dsRED+ cells, and then transplanted into sub-lethally irradiated mice. In order to study the contribution of the f-circM9 to leukemogenesis at the single cell level, transplantations were performed with different cell numbers in a limiting dilution setting, transplanting either 30,000 or 500 cells per recipient. Three weeks post-transplantation, recipient mice were sacrificed, the expression of f-circM9 in leukemic cells was confirmed by PCR, and the progression of leukemia was analyzed. No difference in the number of leukemic blasts was detected in mice transplanted with a high number of leukemic cells. Irrespective of the expression of the f-circM9, mice developed leukemia and displayed similar percentages of leukemic cells within spleen and bone marrow (FIG. 10B). Interestingly however, when transplanted at limiting rate, clear differences could be observed among the recipient mice (FIG. 3F-G). The presence of the f-circM9 favored the progression of the leukemia. Within three weeks, mice that were transplanted with leukemic cells carrying the f-circM9 together with the MLL/AF9 fusion protein (FIG. 3F) showed an enlarged spleen and a higher number of leukemic cells both in the bone marrow and spleen (FIG. 3G).

Together, these experiments show that f-circM9, coupled with other oncogenic stimuli (e.g. the presence of the oncogenic fusion protein), play an active role in favoring leukemia progression in vivo.

### Example 4: f-circRNAs Confer Resistance to Therapy In Vivo

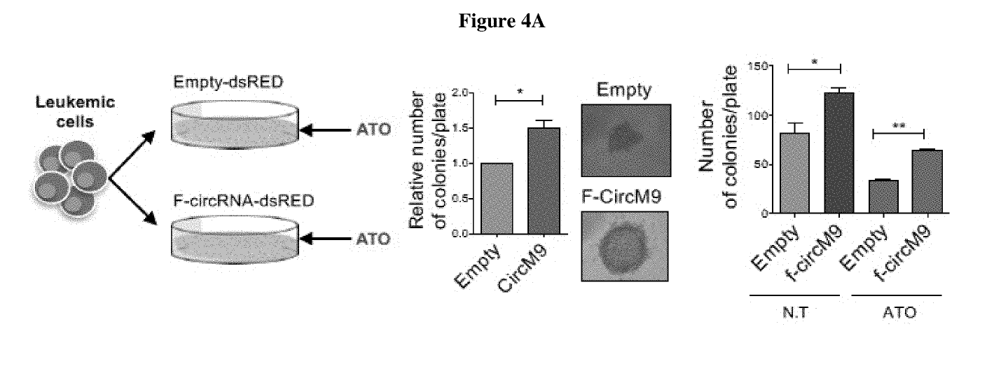

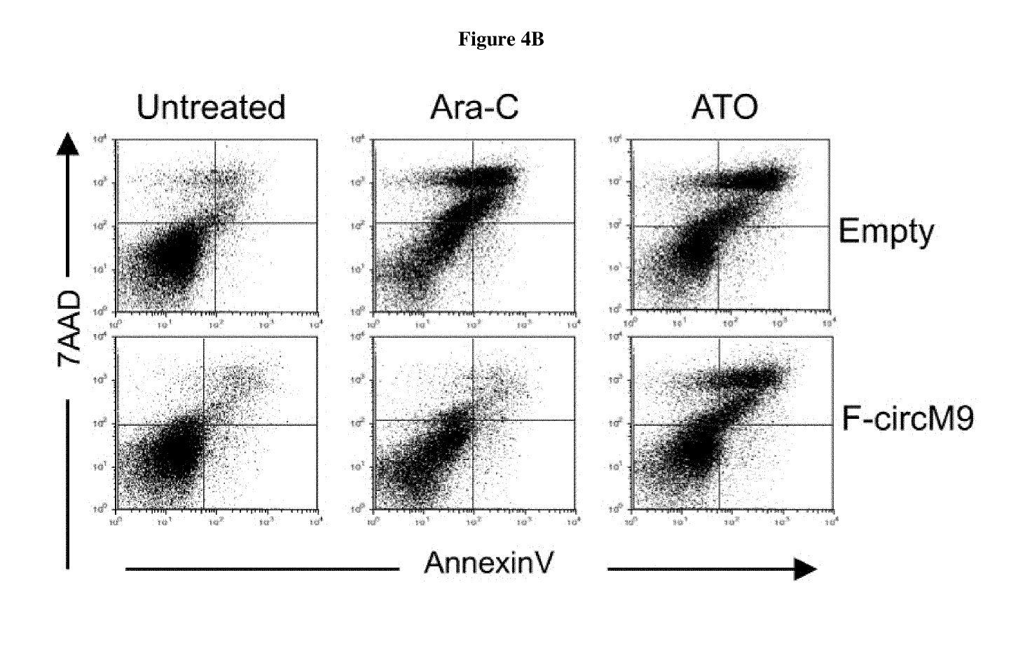

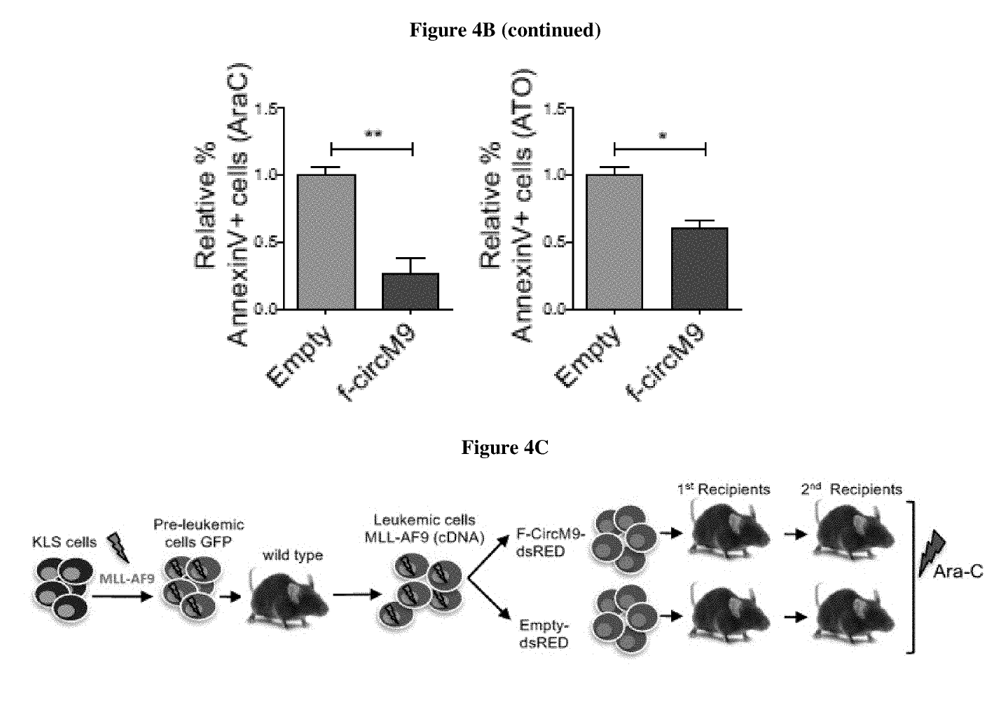

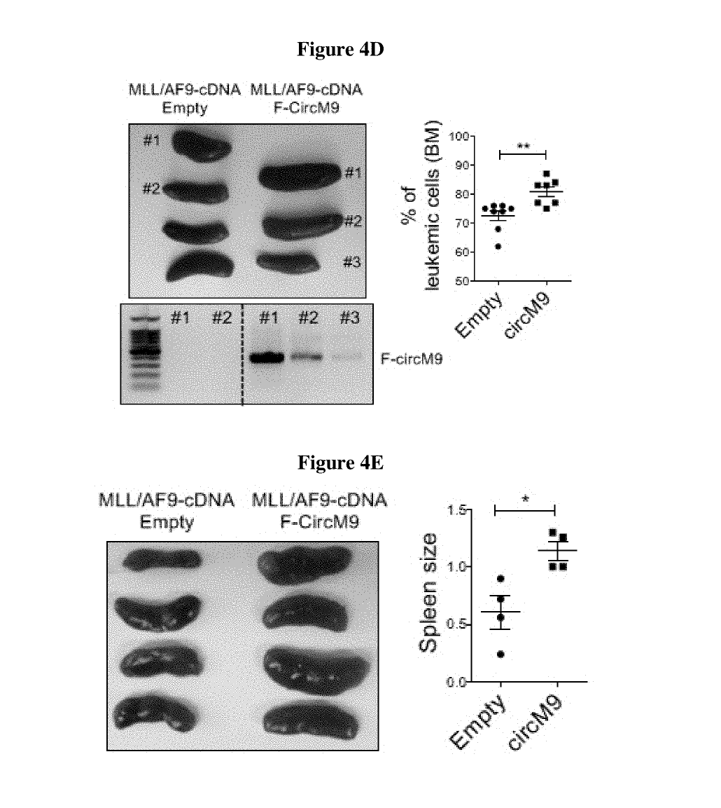

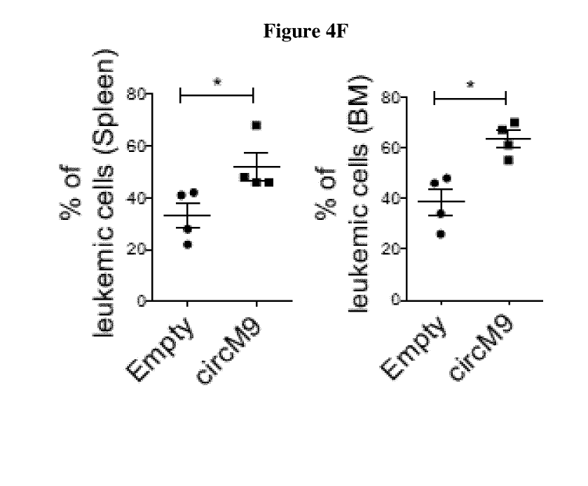

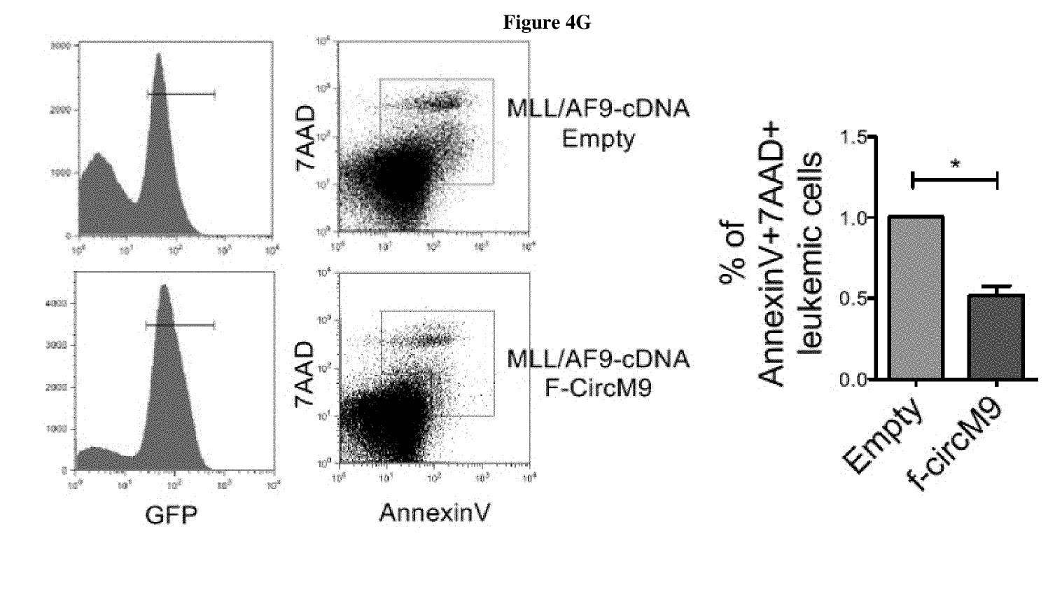

Relapsed disease following remission achieved with conventional therapy still remains one of the central problems in the treatment of leukemia (Forman and Rowe, 2013; Bailey et al., 2008). One major cause of relapse of the leukemia is the development of drug resistance by leukemic cells, and thus loss of responsiveness to the therapeutic regimen (Ding et al., 2012; Zahreddine and Borden, 2013). Since the expression f-circRNAs confers a proliferative advantage to leukemic cells, the possibility that the expression of f-circRNA could confer a survival advantage and protection to cancer cells from drug-induced apoptosis was next investigated. To answer this important question, the MLL/AF9 model was used, and in vitro and in vivo experiments were performed. Leukemic cells carrying the MLL/AF9 fusion protein were transduced with f-circM9 or an empty vector. For the in vitro setting, cells were sorted as dsRED+ and cultured in methylcellulose. Arsenic trioxide (ATO) (Ito et al., 2008), an approved standard-of-care drug for the treatment of leukemia, was then added to the semi-solid medium and left for five days of culture. At the end of the culture period proliferation and colony formation were assessed. As shown in FIG. 4A, the presence of f-circM9 conferred protection to leukemic cells during treatment, and an increase in size and quantity of colonies was observed at the end of the treatment period for cells expressing the f-circM9 as compared to the control vector (FIG. 4A and FIG. 10C).

To corroborate these results, an independent experimental setting and an additional standard-of-care approved drug for leukemia treatment, cytarabine (Ara-C) (Lowenberg et al., 2011; Lengfelder et al., 2012), was used. K562 cells (Lozzio and Lozzio, 1979) were then transduced with viral vectors expressing f-circM9. Cells transduced only with an empty vector were used as control. F-circM9-expressing cells were sorted for a pure population and were subsequently subjected to treatment with Ara-C and ATO. As shown in FIG. 4B, no differences in apoptosis were detected upon the expression of f-circRNAs in untreated cells. On the contrary, clear differences were noticed in treated cells. Cells transduced with an empty vector showed signs of apoptosis upon treatment with Ara-C and ATO, while cells expressing f-circM9 resulted to be markedly protected and displayed reduced signs of apoptosis. These results further demonstrate that f-circM9 can bestow to tumor cells a survival advantage also in response to therapy treatment, hence likely impacting therapeutic outcomes.

Next, to further validate whether f-circM9 could confer protection to leukemic cells under treatment, in vivo experiments were performed. In order to obtain a sufficient number of cells to perform the experiment, leukemic cells expressing either f-circM9 or the empty vector in primary recipients were expanded (FIG. 4C). The expression of the transgene was maintained throughout the entire experiment (FIG. 4D). The same number of leukemic cells were then derived from primary recipients and transplanted into experimental mice (FIG. 4C, secondary recipients). After the onset of leukemia (two weeks post-transplantation), mice were treated with Ara-C according to the data reported in Lowenberg et al. (2011), Lengfelder et al. (2012) and Zuber et al. (2009). Four days post-treatment, mice were sacrificed and the progression of the leukemia was analyzed. As shown in FIG. 4E, mice transplanted with leukemic cells that expressed f-circM9 showed larger spleens compared to mice transplanted with leukemic control cells. Importantly, the number of leukemic cells infiltrating the spleen and present in the bone marrow was higher in the f-circM9 expression setting compared to the control setting (FIG. 4F).

Because the number of surviving cells in the treatment with chemotherapy was higher where the leukemic cells expressed the f-circM9 together with the MLL/AF9 fusion protein, their levels of apoptosis were investigated. To this end, leukemic cells within the bone marrow were stained with AnnexinV-7-AAD. The overall number of apoptotic leukemic cells (GFP+) after treatment was reduced in mice bearing leukemic cells that expressed f-circM9 as compared to the controls (FIG. 4G). These results further demonstrate that f-circRNAs originating from chromosomal translocation are functionally active and can provide tumor cells a survival advantage in response to therapy treatment, likely impacting therapeutic outcomes.

### Example 5: f-circRNAs are Critical for Leukemic Cell Viability

While the experiments performed herein demonstrate a role for f-circRNAs in cellular transformation and tumor progression, whether the expression of the endogenous f-circRNAs is relevant to the survival of leukemic cells that endogenously carry the chromosomal translocation was next assessed. To this end, the expression, the distribution and the stability of one of the two endogenous f-circM9s (f-circM9_1 see also FIG. 1F) were first analyzed. Next, functional experiments knocking down the f-circM9_1 in THP1 cells were performed.

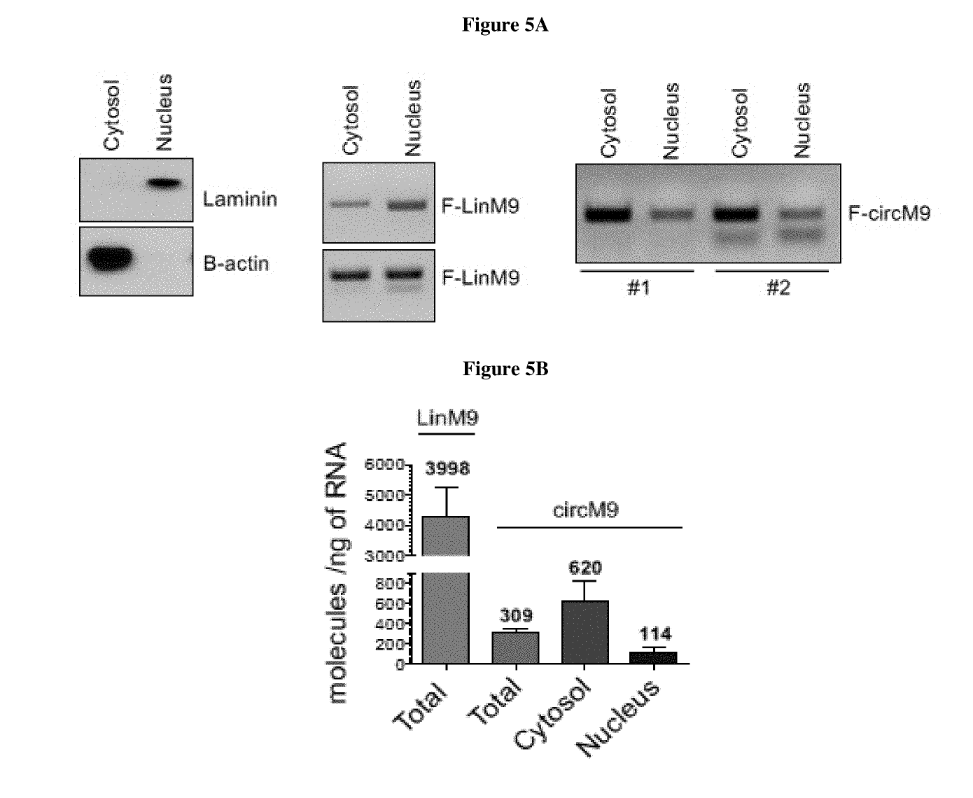

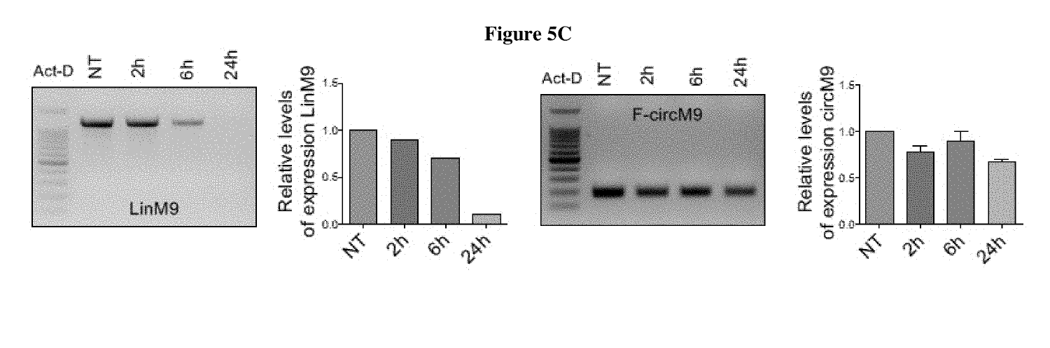

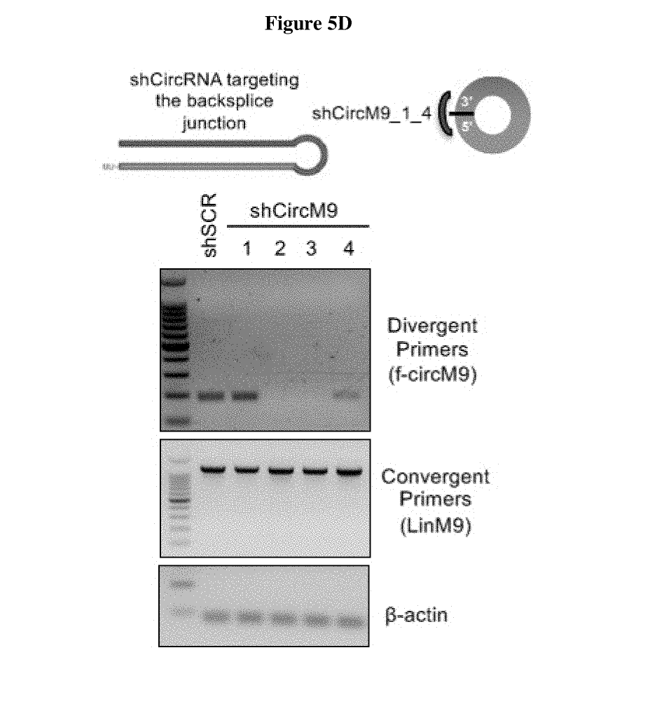

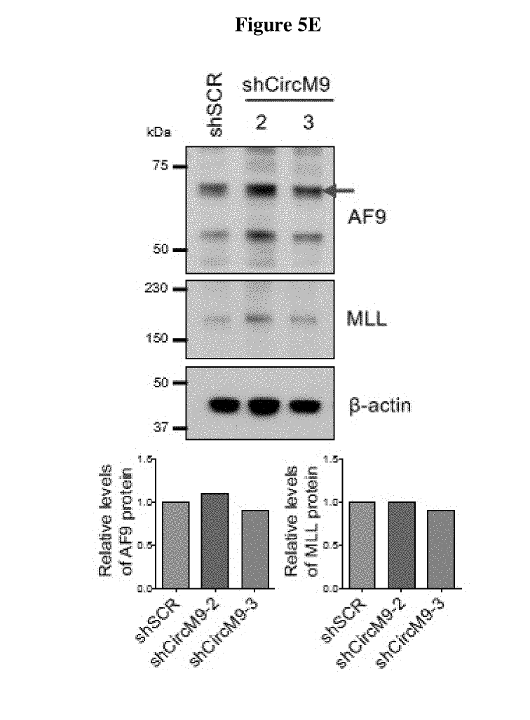

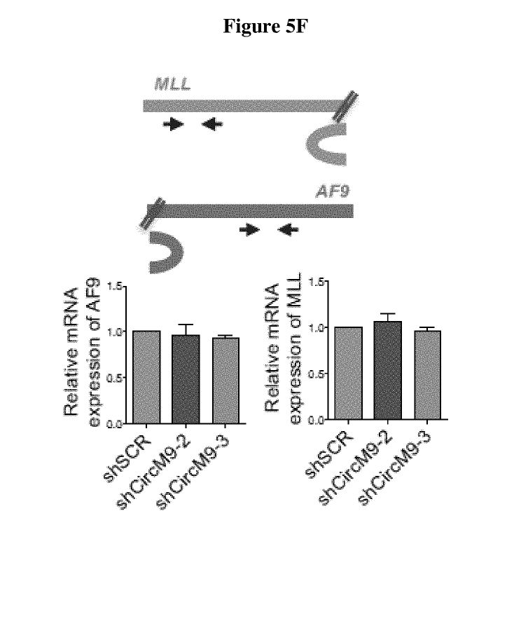

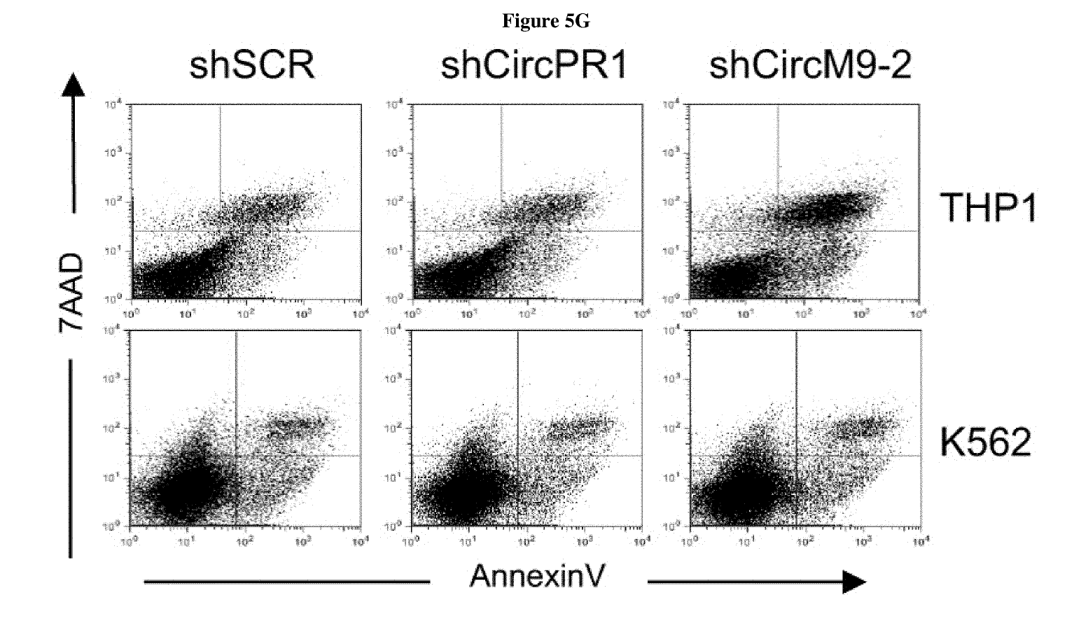

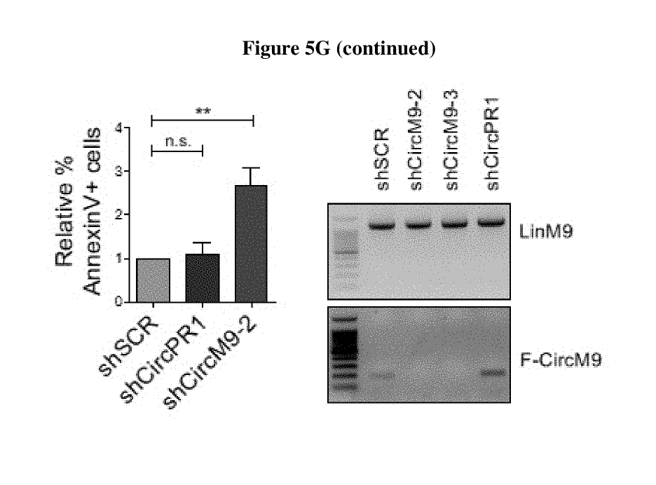

In order to determine the localization of f-circM9_1, cells were fractionated into cytosolic and nuclear fractions and the presence of f-circM9_1 was assessed in each fraction as number of molecules of f-circM9_1 per ng of total RNA (see Methods). These experiments revealed that f-circM9 levels were in the range of expression of many cellular mRNAs (FIG. 5A-C). THP1 cells expressed lower levels of f-circM9_1 compared to its linear-fusion transcript. Interestingly, the presence of f-circM9_1 in both cytosolic and nuclear compartments was observed with enrichment in the cytosol as compared to the nuclear fraction (FIG. 5A-B). Moreover, when compared to its linear transcripts, f-circM9_1 showed higher stability (FIG. 5C), in agreement to what was already reported about circRNAs (Jeck et al., 2013).

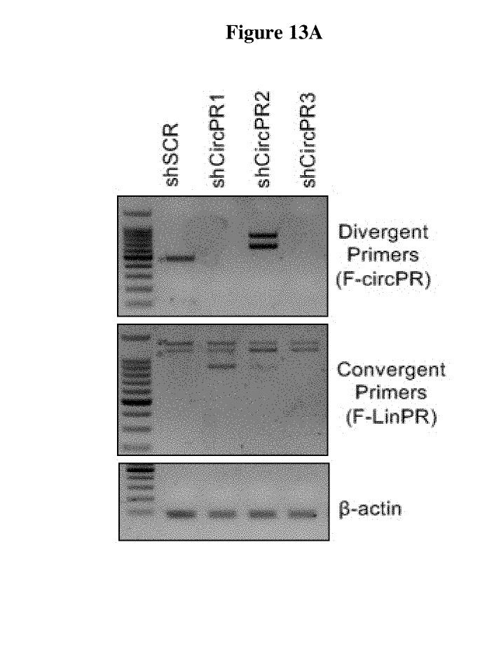

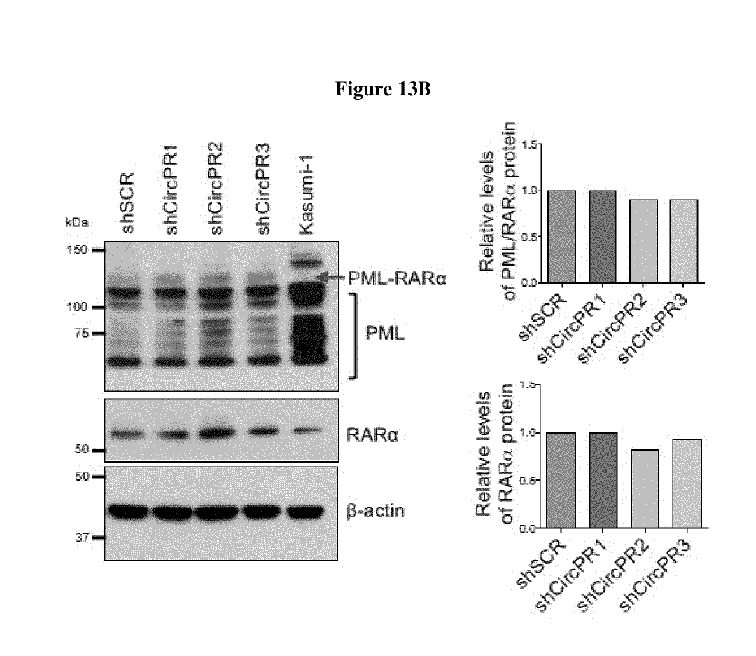

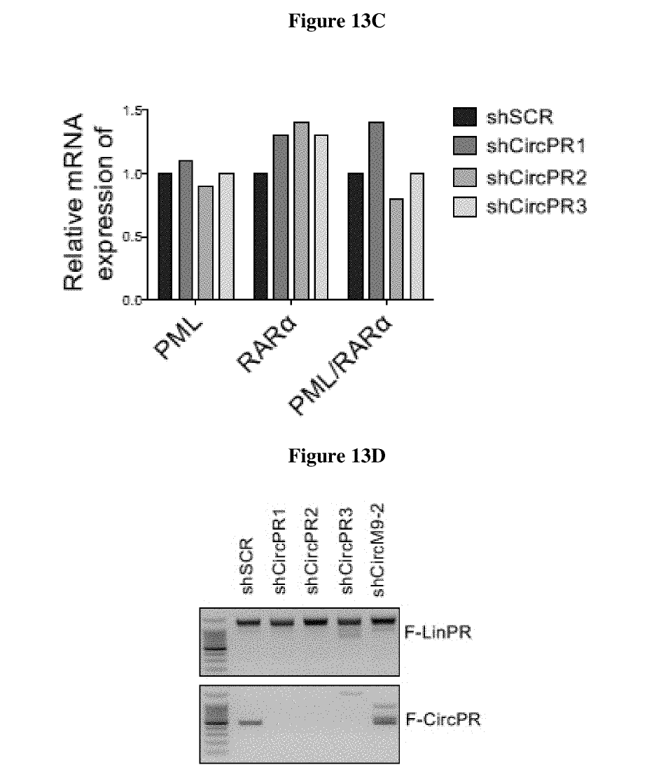

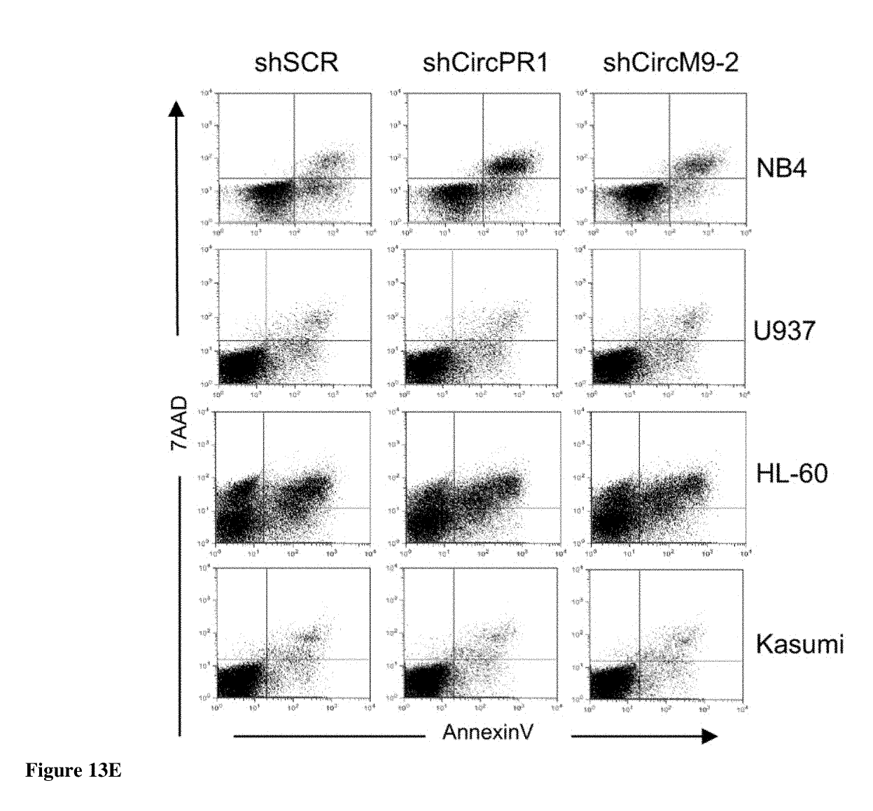

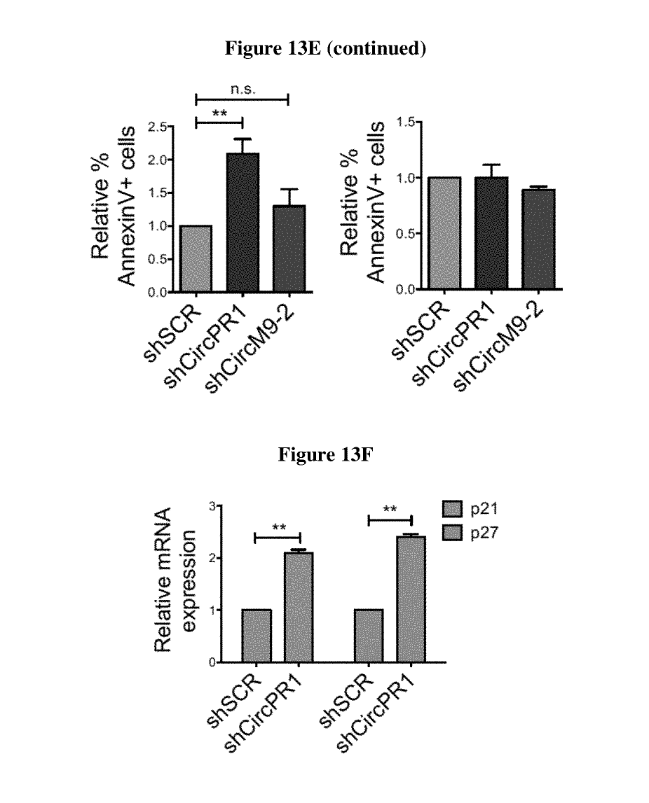

f-circM9_1 was next targeted using shRNAs specifically directed against the back-splice junction. Different shCircRNAs were screened in order to find a candidate that: i) had the ability to knock down the f-circM9_1 (assessed by PCR with divergent primers, FIG. 5D); ii) did not affect the linear transcripts and the MLL and AF9 proteins (assessed by Western blot as shown in FIG. 5E, and by PCR using primers that could selectively recognize the linear transcripts, as shown in FIG. 5F); and iii) had no toxic effects on unrelated cells (assessed by testing the shCircRNAs on leukemic cell lines that did not carry the MLL/AF9 translocation, FIG. 5G and FIG. 13E). Only one of the screened shCircRNAs (shCircM9-2) met all the three criteria, and was therefore used to evaluate the functionality of f-circM9_2 in THP1 cells (FIG. 5G).

Knocking down f-circM9 in THP1 cells resulted in increased apoptosis, as measured with the AnnexinV-7-AAD staining (FIG. 5G). A similar approach was used to identify an shCircRNA effectively targeting f-circPR (FIG. 13A-E). The knockdown of f-circPR in NB4 cells also triggered apoptosis while increasing the expression of p21 and p27 compared to control cells (FIG. 13F). Thus, f-circRNAs play an important role in maintaining the viability of leukemic cells.

### Example 6: Materials and Methods

Cell Lines and Reagents

THP-1, K562, U937, HL-60, Kasumi and NB4 cell lines were maintained in RPMI medium supplemented with 10% FBS. MEFs Arf−/− cells were grown in DMEM supplemented with 10% FBS. Primary leukemic cells transduced with MLL/AF9 cDNA were maintained in StemSpan medium (Stem Cell Technologies) supplemented with IL6 (15 ng/ml), IL3 (15 ng/ml) TPO (50 ng/ml) and SCF (50 ng/ml) during the transduction with retroviral vectors. Cells were grown at 37° C. in a 5% CO₂ humidified incubator. Fractionation of the cells to separate nucleus and cytoplasm has been performed using PARIS-kit (Ambion) following the manufacturer's instructions. The efficiency of the separation of the 2 fractions was assessed by Western blot analysis of β-actin (cytosol) and laminin (nucleus). In selected experiments, cells were treated with Arsenic Trioxide (ATO) or with Cytarabine (Ara-C) at the concentration of 1 μM and 0.5 μM respectively.

Mice

Animal experiments were performed in accordance with the guidelines of Beth Israel Deaconess Medical Center Institutional Animal Care and Use Committee. To develop a MLL/AF9 model of leukemia, hematopoietic stem cells (KLS-cells: ckit+Sca1+Lin-) were FACS-sorted from wild type C57Bl/6 mice, and transduced with a retroviral vector expressing MLL/AF9 cDNA, which carried GFP as a reporter. Transduced cells were then transplanted back into recipients mice sub-lethally irradiated (650 rad). The progression of leukemia was monitored progressively through bleeding. Fully transformed leukemic cells were then extracted from the bone marrow of recipient mice as GFP+ cells and used for further experiments. For limiting-dilution experiments (FIG. 3F-G) leukemic cells were transduced with the retroviral vectors and then re-sorted for dsRED (as represented in FIG. 3A). Sorted cells were then injected in sub-lethally irradiated recipients at a concentration of 500 cells per mouse. As control, a separate cohort of mice received 30,000 cells. Mice were sacrificed three weeks post-transplantation. For treatment experiments, mice were transplanted with leukemic cells; Ara-C was administered for 4 days in a raw, using the concentrations published in (Zuber et al., 2009).

Isolation of RNA and Treatment with RNase R

Total RNA was extracted using TRIZOL® (Invitrogen) according to the manufacturer's instructions. To enrich circRNA isoforms, RNase R treatment was carried out for 15 minutes at 37° C. using 2U RNase-R (Epicentre) per 1 μg of RNA. Treated RNA was directly reverse transcribed using the RETROscript System (Life Technologies) with random decamer primers according to the manufacturer's instructions.

PCR and qRT-PCR Analysis

40 ng of RNA were used for the PCR analysis of the f-circRNAs. The PCR reactions were performed using HotStarTaq Master Mix (Qiagen) for up to 40 cycles. Primer sequences are provided in Table 1.

TABLE 1
Primers used for the PCR analysis.

| PML/RARα Convergent primers | | |
|---|---|---|
| (not targeting the circPML/RARα): | | |
| Fw: | ATGCAGCTGTATCCAAGAAAGC | (SEQ ID NO: 11) |
| Rv: | CTCCGCAGATGAGGCAGAT | (SEQ ID NO: 12) |

PML/RARα Divergent primers:

| | | |
|---|---|---|
| Fw1: | GCTACCACTATGGGGTCAGC | (SEQ ID NO: 13) |
| Rv1: | CGTTGTATTGGAGACATCCTCTC | (SEQ ID NO: 14) |
| Fw2: | GTGTCACCGGGACAAGAACT | (SEQ ID NO: 15) |
| Rv2: | CTGGGCCTTCACTCTCTCTG | (SEQ ID NO: 16) |

MLL/AF9 Convergent primers:

| | | |
|---|---|---|
| Fw1: | GTGTTGTGAAGAACGTGGTGG | (SEQ ID NO: 17) |
| Rv1: | CGGCTGCCTCCTCTATTTACA | (SEQ ID NO: 18) |

(not targeting the circMLL/AF9)

| | | |
|---|---|---|
| Fw2: | GAGGACCCCGGATTAAACAT | (SEQ ID NO: 19) |
| Rv2: | TGGTGGAGGTTCGTGATGTA | (SEQ ID NO: 20) |

MLL/AF9 Divergent primers:

| | | |
|---|---|---|
| Fw: | TCTGAACAACCCAGTCCTGC | (SEQ ID NO: 21) |
| Rv: | TGGTGGAGGCTGCTTTTTCT | (SEQ ID NO: 22) |

Spanning the junction of the circM9_2

| | | |
|---|---|---|
| Fw: | CCAGGCAACAAGACAAGTCATC | (SEQ ID NO: 23) |
| Rv: | GCTTCTTTATATTGCGACCACCA | (SEQ ID NO: 24) |

EWS/FLI1 Convergent primers:

| | | |
|---|---|---|
| Fw: | ATCCTACAGCCAAGCTCCAA | (SEQ ID NO: 25) |
| Rv: | GGACTTTTGTTGAGGCCAGA | (SEQ ID NO: 26) |

EWS/FLI1 Divergent primers:

| | | |
|---|---|---|
| Fw: | TGGCCTCAACAAAAGTCCTC | (SEQ ID NO: 27) |
| Rv: | TTGGAGCTTGGCTGTAGGAT | (SEQ ID NO: 28) |

EML4/ALK1 Divergent primers:

| | | |
|---|---|---|
| Fw: | GCAGAGCCCTGAGTACAAGC | (SEQ ID NO: 29) |
| Rv: | CCACAGCCAAAACAACTTCA | (SEQ ID NO: 30) |

EML4/ALK1 Convergent primers:

| | | |
|---|---|---|
| Fw: | CACACCTGGGAAAGGACCTA | (SEQ ID NO: 31) |
| Rv: | TGCCAGCAAAGCAGTAGTTG | (SEQ ID NO: 32) |

f-circM9 Quantification primers:

| | | |
|---|---|---|
| Fw: | CCAGAGCAGAGCAAACAGA | (SEQ ID NO: 33) |
| Rv: | TTTCACAGCTTGTTGCC | (SEQ ID NO: 34) |

LinM9 Quantification primers:

| | | |
|---|---|---|
| Fw: | CCCAAGAAAAAGCAGCCT | (SEQ ID NO: 35) |
| Rv: | TGGTCTGGGATGGTGTGAA | (SEQ ID NO: 36) |

PCR products were visualized after electrophoresis in a 1.5% ethidium bromide-stained agarose gel. For sequencing, PCR products were cut from the gel and purified using QIAquick gel extraction kit (Qiagen) according to the manufacturer's instructions. In selected experiments, qRT-PCRs were performed in order to quantify the expression of the f-circRNAs. qRT-PCRs were carried out using SybrGreen reaction mix and StepOnePlus real-time PCR system (Applied Biosystems). f-circM9 and LinM9 were quantified by PCR in respect to their standard curve. In order to generate the standard curve, the PCR products containing the back-splice junction (for f-circM9) or the break point (for the LinM9) were generated using the respective primers in Tables 2 and 3, and cloned using the pGEM-T easy Vector System II (Promega), by following the manufacturer instruction. The number of molecules per ng of RNA was then calculated by quantifying the PCR bands, and by plotting them to the serially diluted standard curve.

Western Blots

For Western blots, cell lysates were prepared with RIPA buffer. The following antibodies were used: rabbit anti-PML clone H-238 (Santa Cruz), rabbit polyclonal anti-RARα (Santa Cruz), mouse polyclonal anti-β-actin (Sigma-Aldrich), mouse monoclonal anti-HSP90 (BD Biosciences), anti-AF9 (Bethyl), anti-MLL (Bethyl), anti-Akt (Cell Signaling), anti-pAkt (S473) (Cell Signaling), anti-ERK1/2 (Cell Signaling), anti-pERK1/2 (Cell Signaling).

Cloning of PML/RARα f-circRNA

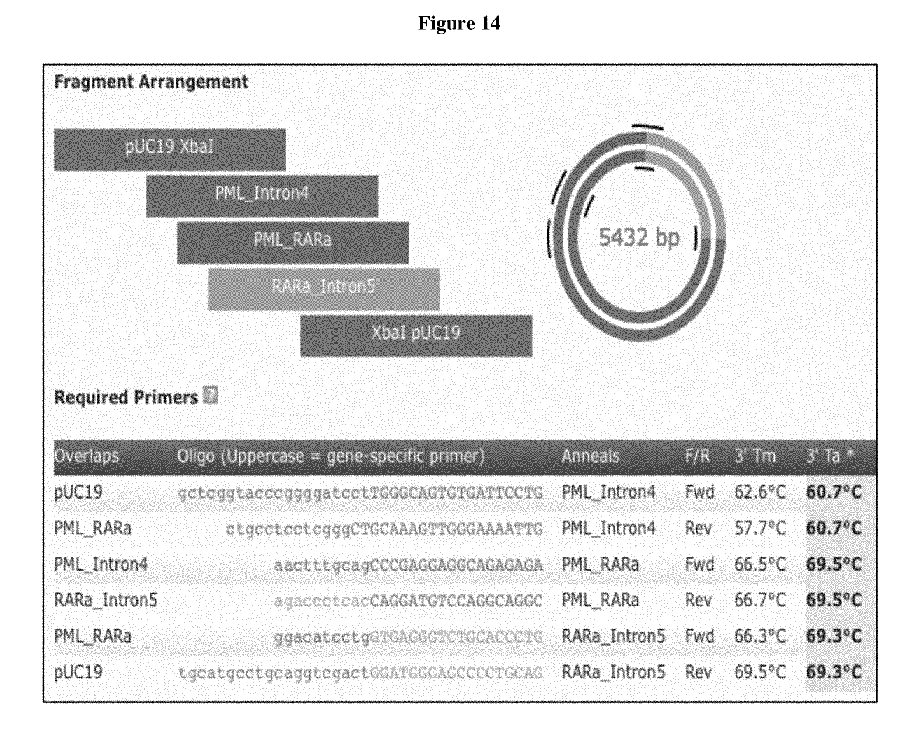

Cloning of the fusion circRNA was carried out using the Gibson Assembly Cloning Kit (New England Biosciences, NEB) with an intermediate sub-cloning of the insert into the pUC19 plasmid. The intronic sequences were obtained by PCR reaction with 200 ng of genomic DNA as template. The exonic sequences were obtained by PCR from cDNA. The insert was then moved to a retroviral vector pCMMP-MCS-IRES-mRFP (Addgene). Maps and sequences of the overlapping primers are provided in the FIG. 14. The insert was removed from the pUC19 vector by restriction digest with EcoRI and SphI (NEB), the retroviral vector was opened by digestion with XhoI (NEB). All restriction digests were carried out for 1 hour at 37° C. with 1 μL enzyme per μg RNA, restriction enzymes were heat inactivated at 65° C. for 20 minutes. Blunt ends were generated in the insert and vector by incubation with Klenow DNA Polymerase (NEB) for 15 minutes at room temperature. After gel purification the insert was ligated into the vector with Quick Ligation Kit (NEB) following the manufacturer's instructions. Correct insertion in the retroviral vector was validated by restriction digest and sequencing.

Cloning of MLL/AF9 f-circRNA

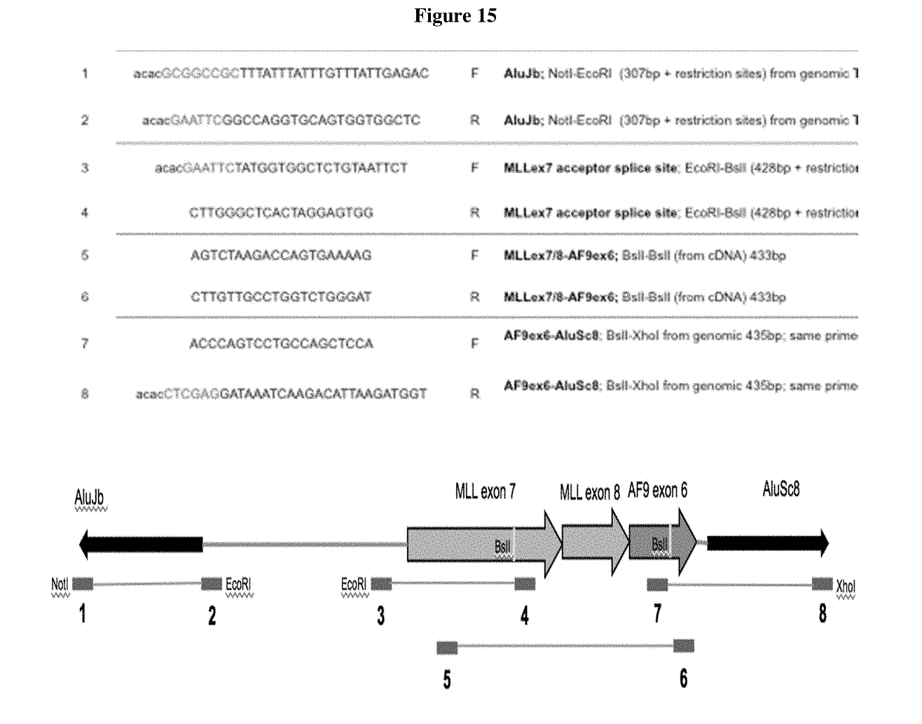

In order to overexpress the circular RNA MLL exon 7-8, AF9 exon 6, a vector including the flanking inverse Alu sequences was generated. The genomic regions for the AluSx of MLL intron 6, MLL exon7 splice acceptor, AF9 exon 6 splice donor and the AluSc8 of AF9 intron 6 were amplified from THP1 genomic DNA using the primers in FIG. 15. The region including MLL exon 7-8 and AF9 exon 6 was amplified from THP1 cDNA. The PCR fragments were digested according to the restriction sites indicated in Table 12, and used as template to amplify the entire region by using the primers 1 and 4 in FIG. 15. The full-length PCR fragment was digested NotI-XhoI and cloned into the pCMMP-MCS-IRES-mRFP vector.

TABLE 12
PCR primers used for cloning f-circM9.

| | |
|---|---|
| acacGCGGCCGCTTTATTTA | AluJb; NotI-EcoRI (307 bp + |
| TTTGTTTATTGAGAC | restriction sites) from |
| (SEQ ID NO: 37) | genomic THP-1 |
| acacGAATTCGGCCAGGTGC | AluJb; NotI-EcoRI (307 bp + |
| AGTGGTGGCTC | restriction sites) from |
| (SEQ ID NO: 38) | genomic THP-1 |
| acacGAATTCTATGGTGGCT | MLLex7 acceptor splice |
| CTGTAATTCT | site; EcoRI-BslI (428 bp + |
| (SEQ ID NO: 39) | restriction sites) from |
| | genomic THP-1 |
| CTTGGGCTCACTAGGAGTGG | MLLex7 acceptor splice |
| (SEQ ID NO: 40) | site; EcoRI-BslI (428 bp + |
| | restriction sites) from |
| | genomic THP-1 |
| AGTCTAAGACCAGTGAAAAG | MLLex7/8-AF9ex6; BslI-BslI |
| (SEQ ID NO: 41) | (from cDNA) 433 bp |
| CTTGTTGCCTGGTCTGGGAT | MLLex7/8-AF9ex6; BslI-BslI |
| (SEQ ID NO: 42) | (from cDNA) 433 bp |
| ACCCAGTCCTGCCAGCTCCA | AF9ex6-AluSc8; BslI-XhoI |
| (SEQ ID NO: 43) | from genomic 435 bp; same |
| | primers for MLLex5-6-7- |
| | AF9ex6 |
| acacCTCGAGGATAAATCAA | AF9ex6-AluSc8; BslI-XhoI |
| GACATTAAGATGGT | from genomic 435 bp; same |
| (SEQ ID NO: 44) | primers for MLLex5-6-7- |
| | AF9ex6 |

Mutagenesis of Retroviral Vectors

Both f-circM9- and f-circPR-expressing retroviral vectors were subjected to mutagenesis at the splicing-donor site. Mutagenesis was performed using the QuikChange XL Site-Directed mutagenesis kit (Agilent), as represented in FIG. 2G.

Cloning of shCircRNAs Targeting the Back-Splice Junction of f-circRNAs

Plko.1-TCR cloning vectors (Addgene 10878) were used to express shRNAs targeting the back-splice junctions of the f-circRNAs. Vectors were cloned following the instructions at the weblink addgene.org/tools/protocols/plko/. A scramble shRNA was used as control.

### Proliferation Assays, Crystal Violet Staining and Focus Formation Assay

MEFs were derived from p19-Arf−/− mice, and immortalized in culture by culturing for several passages. For the proliferation assay, cells were seeded at a concentration of 5×104 cells per well in a 12-wells plate and cultured for 5 days in complete medium (DMEM+10% FBS) at 37° C. Cells were then fixed every day and stained with crystal violet. For the focus formation assay, cells were seeded at a concentration of 5×104 cells per well in a 12-wells plate, and the cultures were carried on for 10 days without changing media. Once formed, the visible cell foci were fixed with 10% formalin for 30 minutes and subsequently stained with crystal violet. Crystal violet was then solubilized with 10% acetic acid and their absorbance was measured.

Flow-Cytometry Analysis of Annexin-V+ Cells

Apoptotic cells were analyzed with 7AAD and Annexin-V-APC. Cells were stained for 15 minutes with both markers and then analyzed using LRSII (BD, Pharmingen).

Production of Viral Particles and Infection of Target Cells

All the viral particles were produced in transfecting 293T cells using Lipofectamine (Invitrogen). Retroviral vectors (backbone: pCMMP-MCS-IRES-mRFP) were co-transfected with pECO or pAmpho packaging plasmids, while lentiviral vectors (plko.1 for shCircRNAs) were co-transfected with packaging vectors of second generation. 48 hours after transfection, the viral supernatant was collected; viral particles were concentrated through centrifugation and used to transduce target cells with polybrene.

Bioinformatics Analysis—Data Acquisition

Total RNA-seq data from the THP1 cell line was downloaded from the Gene Expression Omnibus (GEO) (Barrett et al., 2013) under accession number GSM1125252. Total RNA-seq data of primary patients with acute leukemia harboring the MLL/AF9 fusion gene (6 patients, 12 samples) or PML/RARα fusion gene (14 patients, 28 samples) were downloaded from Cancer Genomic Hub (CGHub) (Wilks et al., 2014), and the fusion genes were identified using the cBioPortal (Cerami et al., 2012). The details of the data set are summarized in Table 13.

TABLE 13

| Parameters | | | | | | | | | | |
|---|---|---|---|---|---|---|---|---|---|---|
| MA = MLL/AF9, | | | | | | | | | | |
| PR = PML/RARα | | | | | Circular | | Linear | | | |
| Sam- | | | | | MA | PR | MA | | PR | |
| ples | Fusion | n | l | w | Gen | Gen | Exp | Gen | Exp | Gen |
| JG01 | MLL/ | 1 | 42 | 30 | 0 | 0 | 2 | 0 | 0 | 0 |
| | AF9 | | | | | | | | | |
| JG02 | PML/ | 1 | 44 | 30 | 1 | 1 | 0 | 0 | 0 | 1 |
| | RARα | | | | | | | | | |
| JG03 | PML/ | 1 | 40 | 25 | 0 | 0 | 0 | 0 | 0 | 0 |
| | RARα | | | | | | | | | |
| JG06 | PML/ | 1 | 42 | 30 | 0 | 0 | 0 | 0 | 0 | 1 |
| | RARα | | | | | | | | | |
| JG07 | PML/ | 1 | 40 | 30 | 0 | 4 | 0 | 0 | 0 | 5 |
| | RARα | | | | | | | | | |
| JG08 | PML/ | 2 | 41 | 30 | 0 | 0 | 0 | 0 | 0 | 1 |
| | RARα | | | | | | | | | |
| JG09 | PML/ | 1 | 44 | 30 | 0 | 0 | 0 | 0 | 0 | 3 |
| | RARα | | | | | | | | | |
| JG11 | | 2 | 42 | 35 | 0 | 0 | 0 | 0 | 0 | 0 |
| JG12 | PML/ | 1 | 44 | 30 | 0 | 4 | 0 | 0 | 0 | 15 |
| | RARα | | | | | | | | | |

Bioinformatics Analysis—Reference Library Preparation

Reference library for mapping linear fusions: linear fusion sequences for MLL/AF9 and PML/RARα were obtained from the NCBI Nucleotide Database with accession number EF406122 and AB067754, respectively.

Reference library for mapping f-circRNAs: information on exons involved in the f-circRNAs was obtained from the sequencing of circRNA fusion transcripts in cell lines. Two types of f-circRNA reference libraries were created for mapping potential f-circRNA transcripts from the human RNA-Seq data. The first reference library contained the exact sequences received from the sequencing of circRNA fusions in cell lines (experimental library). For the second reference library, the information on exons involved in f-circRNAs obtained from the experimental library was used, and expected f-circRNA transcripts were then reconstructed based on the most common isoforms using RNA sequence data from the UCSC hg19 transcriptome.

Generation of shRNAs for Targeting the Back-Splice Junction of f-circRNAs

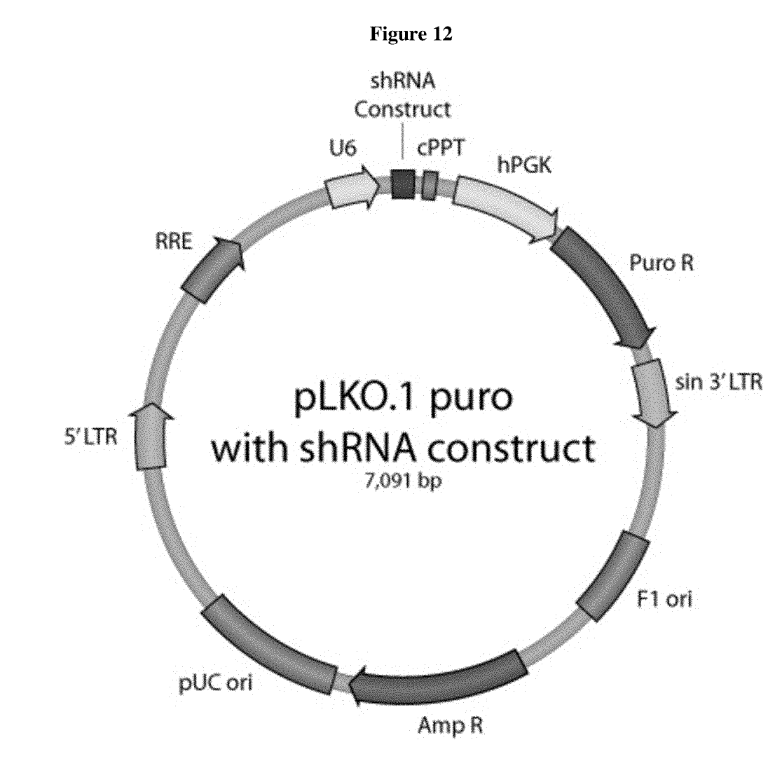

ShRNAs (21 bp long) which selectively target the back-splice junction of f-circRNAs (labeled therefore as shCircRNAs) were cloned into the lentiviral backbone vector pLKO.1 (FIG. 12), following the provided guidelines. shRNAs were designed accordingly to these following rules as listed at the weblink addgene.org/tools/protocols/plko/: 1) starting at 25 nt downstream of the start codon (ATG), search for 21 nt sequences that match the pattern AA(N 19)—if no suitable match is found, search for NAR(N 17)YNN, where N is any nucleotide, R is a purine (A,G), and Y is a pyrimidine (C,U); 2) G-C content should be 36-52%; 3) sense 3′ end should have low stability—at least one A or T between position 15-19; 4) avoid targeting introns; and 5) avoid stretches of 4 or more nucleotide repeats, especially repeated Ts because polyT is a termination signal for RNA polymerase III.

In order to design highly specific shRNAs, publicly available Selector tools were used. In particular, the weblink at broadinstitute.org/rnai/public/seq/search was used. As a brief summary: 1) a sequence of 30 bp (15 bp up-stream and 15 bp down-stream of the back-splice junction) was provided as input for the selector tool; 2) as output, a list of several possible shRNAs was returned, 21 bp in length, all with different starting positions (sequences were listed according to their “intrinsic score,” which takes into account the performance in targeting the specific sequence, and the ability to clone into the pLKO.1 vector—the higher the intrinsic score, the better the chance of cloning a high performing shRNA); 3) according to the starting point of the input sequence and the intrinsic score, sequences were selected that had less than 14 bp shifted to one side of the junction (by choosing these sequences, any shRNAs were excluded that might target linear transcripts other than the f-circRNA); and 4) selected sequences were analyzed with the NCBI's BLAST program in order to predict possible off-target effects (sequences with 100% similarity to unrelated transcripts were excluded).

### Example 7: Discussion

Genomic alterations, particularly aberrant chromosomal translocations, are responsible for the onset of many types of cancers, i.e., leukemias as well as solid tumors (Rabbitts, 1994; Bunting and Nussenzweig, 2013). How such rearrangements can lead to tumorigenesis has been explained by their ability to encode and express oncogenic proteins. However, whether such alterations of the genome could also simultaneously impact the non-coding RNA dimension has not yet been explored. Whether chromosomal translocations could affect the circRNA's dimension of cancer cells has been investigated as described above. The analysis described herein revealed several important conclusions:

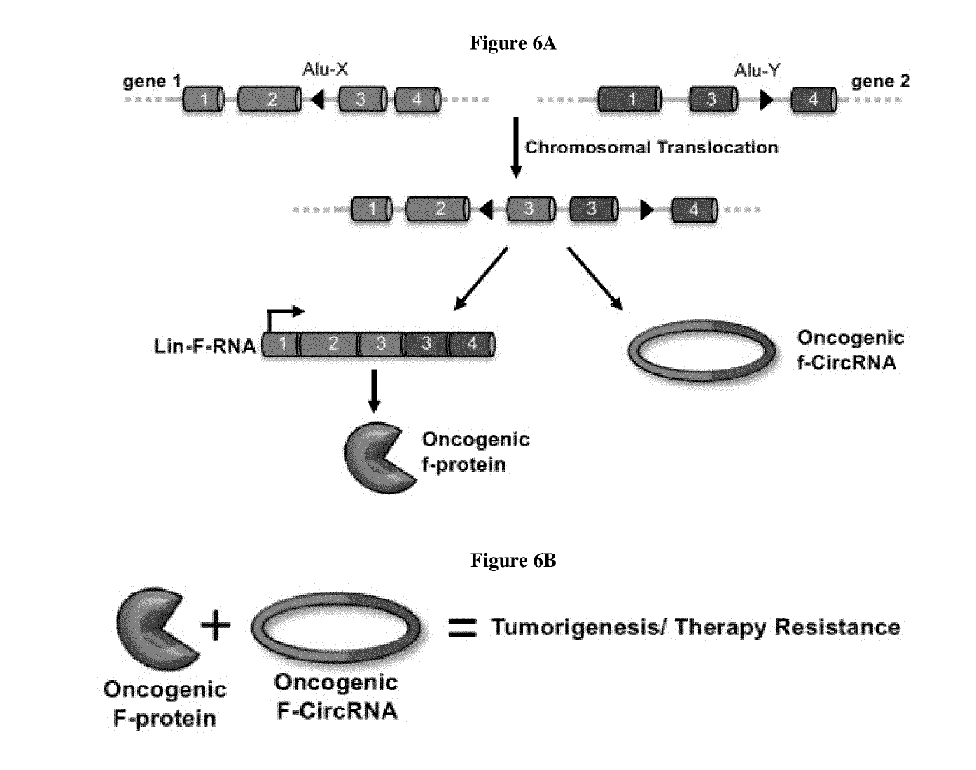

i) Chromosomal translocations generate a new aberrant RNA molecular entity: f-circRNAs. F-circRNA's biogenesis is favored by the juxtaposition of otherwise separated complementary intronic sequences up- and down-stream of the breakpoint region of the translocation (FIG. 6A). According to the presence of such complementary regions within the introns that are located next to the breakpoint, all of the fused primary mRNAs derived from chromosomal translocations, in frame or out of frame, may potentially give rise to f-circRNAs (Jeck et al., 2013). Furthermore, as described for circRNAs, multiple combinations of circularizing exons can be generated from the same fusion gene. Shown herein are examples of fusion circRNAs generated from PML/RARα, MLL/AF9, EWSR1-FLI1 and EML4/ALK1 translocations. However, the formation of f-circRNAs could represent a general mechanism, applicable to many other translocations, occurring in leukemia, solid tumors and non-malignant diseases. f-circRNAs can be also generated from the multitude of out-of-frame chromosomal translocations observed in human cancer. On this basis, high-throughput bioinformatics platforms should be developed to identify of f-circRNAs derived from all known chromosomal translocations.

Notably, recent reports have shown that circRNAs can be stably secreted in exosomes (Li et al., 2015c), and therefore utilized as diagnostic and prognostic markers for cancer (Salzman et al., 2012; Li et al., 2015b; Bahn et al., 2015). In this respect, f-circRNAs in secreted exosomes can be used as pathognomonic markers for many tumor types, especially for those whose biopsies are difficult to obtain. Accordingly, provided herein is a method of diagnosing a subject with a tumor comprising detecting in the subject the presence of a fusion-circular RNA (f-circRNA). Also provided herein is a method of prognosing a tumor type comprising detecting in the tumor the presence of a fusion-circular RNA (f-circRNA). Also provided herein is a method of prognosing a tumor type in a subject comprising detecting in the tumor in the subject the presence of a fusion-circular RNA (f-circRNA). In an embodiment, a change in the presence of the f-circRNA is detected.

Additionally, although not investigated in the present work, chromosomal translocation events can also “knockout” circRNAs normally generated from the wild-type loci involved in the translocation, when these circRNAs encompass the breakpoint region. A deeper investigation of these “inactivating” events and their functional consequences could be just as informative in revealing new tumor suppressive players involved in the onset and progression of the disease. Accordingly, provided herein is a method of determining a tumor suppressor comprising detecting the presence of circRNA in the tumor. In an embodiment, a change in the presence of the circRNA is detected.

ii) Aberrant f-circRNAs are functionally relevant during cancer development and are tumor promoting. f-circRNAs, as demonstrated herein, together with other oncogenic hits, are able to increase cellular proliferation rate, and contribute to cellular transformation and tumorigenesis both in vitro as well as in vivo. These findings uncover a new layer of complexity underlying the role of chromosomal translocations in human disease pathogenesis whereby the biological outputs, in this case the oncogenic signals, might be not be solely limited to the encoded fusion-proteins. In this “multiple hits in one” scenario, the concomitant expression in tumor cells of fusion mRNAs, fusion-proteins and f-circRNAs could be in some instances critical for tumor onset and progression (FIG. 6B). These findings have a number of relevant corollary implications. First, experimental models that mimic the presence of the translocation only by the expression of the related protein might not completely phenocopy the human disease. Thus, in such models, biological mechanisms relevant to tumorigenesis might be missed. Second, and importantly, in all of the cases of out-of-frame chromosomal translocations the generation of f-circRNAs could represent a functionally relevant oncogenic element, leading to tumor development independent of the fusion-protein.

iii) The expression of f-circRNAs in cancer cells is essential for their maintenance and confers resistance to therapy. It has been demonstrated herein that upon silencing of endogenous f-circRNAs, cancer cells undergo apoptosis while f-circRNAs expression protects cancer cells from drug-induced apoptosis. On this basis, pharmaceutical interventions that are aimed at blocking f-circRNAs or their downstream effectors could prove beneficial when paired with conventional therapies to eradicate the disease. Accordingly, provided herein are methods for inhibiting the expression of f-circRNA in a subject in need thereof, comprising administering an f-circRNA inhibiting agent and/or comprising inhibiting agent one or more downstream effectors of f-circRNA.

Collectively, the work described herein demonstrates, for the first time, the existence of a new aberrant molecular species, f-circRNAs, and their relevance to human disease. These observations expand on the current knowledge about the onset and progression of cancer and unravel new possible opportunities for therapeutic intervention. The systematic identification and characterization of f-circRNAs represent an important milestone in the understanding of the genetic and molecular basis of cancer development.

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

### REFERENCES

Anderson, J. L., Denny, C. T., Tap, W. D., and Federman, N. (2012). Pediatric sarcomas: translating molecular pathogenesis of disease to novel therapeutic possibilities. Pediatr Res 72, 112-121.
Ashwal-Fluss, R., Meyer, M., Pamudurti, N. R., Ivanov, A., Bartok, O., Hanan, M., Evantal, N., Memczak, S., Rajewsky, N., and Kadener, S. (2014). circRNA biogenesis competes with pre-mRNA splicing. Mol Cell 56, 55-66.
Bahn, J. H., Zhang, Q., Li, F., Chan, T. M., Lin, X., Kim, Y., Wong, D. T., and Xiao, X. (2015). The landscape of microRNA, Piwi-interacting RNA, and circular RNA in human saliva. Clin Chem 61, 221-230.
Bailey, L. C., Lange, B. J., Rheingold, S. R., and Bunin, N. J. (2008). Bone-marrow relapse in paediatric acute lymphoblastic leukaemia. Lancet Oncol 9, 873-883.
Barrett, S. P., Wang, P. L., and Salzman, J. (2015). Circular RNA biogenesis can proceed through an exon-containing lariat precursor. Elife 4.
Barrett, T., Wilhite, S. E., Ledoux, P., Evangelista, C., Kim, I. F., Tomashevsky, M., Marshall, K. A., Phillippy, K. H., Sherman, P. M., Holko, M., et al. (2013). NCBI GEO: archive for functional genomics data sets-update. Nucleic Acids Res 41, D991-995.
Bunting, S. F., and Nussenzweig, A. (2013). End-joining, translocations and cancer. Nat Rev Cancer 13, 443-454.
Calin, G. A., and Croce, C. M. (2007). Chromosomal rearrangements and microRNAs: a new cancer link with clinical implications. J Clin Invest 117, 2059-2066.
Cerami, E., Gao, J., Dogrusoz, U., Gross, B. E., Sumer, S. O., Aksoy, B. A., Jacobsen, A., Byrne, C. J., Heuer, M. L., Larsson, E., et al. (2012). The cBio cancer genomics portal: an open platform for exploring multidimensional cancer genomics data. Cancer Discov 2, 401-404.
Cheetham, S. W., Gruhl, F., Mattick, J. S., and Dinger, M. E. (2013). Long noncoding RNAs and the genetics of cancer. Br J Cancer 108, 2419-2425.
Chin, L., Hahn, W. C., Getz, G., and Meyerson, M. (2011). Making sense of cancer genomic data. Genes Dev 25, 534-555.
Conn, S. J., Pillman, K. A., Toubia, J., Conn, V. M., Salmanidis, M., Phillips, C. A., Roslan, S., Schreiber, A. W., Gregory, P. A., and Goodall, G. J. (2015). The RNA binding protein quaking regulates formation of circRNAs. Cell 160, 1125-1134.
Croce, C. M. (2009). Causes and consequences of microRNA dysregulation in cancer. Nat Rev Genet 10, 704-714.
Dekking, E. H., van der Velden, V. H., Varro, R., Wai, H., Bottcher, S., Kneba, M., Sonneveld, E., Koning, A., Boeckx, N., Van Poecke, N., et al. (2012). Flow cytometric immunobead assay for fast and easy detection of PML-RARA fusion proteins for the diagnosis of acute promyelocytic leukemia. Leukemia 26, 1976-1985.
Ding, L., Ley, T. J., Larson, D. E., Miller, C. A., Koboldt, D. C., Welch, J. S., Ritchey, J. K., Young, M. A., Lamprecht, T., McLellan, M. D., et al. (2012). Clonal evolution in relapsed acute myeloid leukaemia revealed by whole-genome sequencing. Nature 481, 506-510.
Dos Santos, G. A., Kats, L., and Pandolfi, P. P. (2013). Synergy against PML-RARα: targeting transcription, proteolysis, differentiation, and self-renewal in acute promyelocytic leukemia. J Exp Med 210, 2793-2802.
Forman, S. J., and Rowe, J. M. (2013). The myth of the second remission of acute leukemia in the adult. Blood 121, 1077-1082.
Greuber, E. K., Smith-Pearson, P., Wang, J., and Pendergast, A. M. (2013). Role of ABL family kinases in cancer: from leukaemia to solid tumours. Nat Rev Cancer 13, 559-571.
Guo, J. U., Agarwal, V., Guo, H., and Bartel, D. P. (2014). Expanded identification and characterization of mammalian circular RNAs. Genome Biol 15, 409.
Hansen, T. B., Jensen, T. I., Clausen, B. H., Bramsen, J. B., Finsen, B., Damgaard, C. K., and Kjems, J. (2013). Natural RNA circles function as efficient microRNA sponges. Nature 495, 384-388.
Hsu, M. T., and Coca-Prados, M. (1979). Electron microscopic evidence for the circular form of RNA in the cytoplasm of eukaryotic cells. Nature 280, 339-340.
Ito, K., Bernardi, R., Morotti, A., Matsuoka, S., Saglio, G., Ikeda, Y., Rosenblatt, J., Avigan, D. E., Teruya-Feldstein, J., and Pandolfi, P. P. (2008). PML targeting eradicates quiescent leukaemia-initiating cells. Nature 453, 1072-1078.
Jeck, W. R., and Sharpless, N. E. (2014). Detecting and characterizing circular RNAs. Nat Biotechnol 32, 453-461.
Jeck, W. R., Sorrentino, J. A., Wang, K., Slevin, M. K., Burd, C. E., Liu, J., Marzluff, W. F., and Sharpless, N. E. (2013). Circular RNAs are abundant, conserved, and associated with ALU repeats. RNA 19, 141-157.
Karreth, F. A., Reschke, M., Ruocco, A., Ng, C., Chapuy, B., Leopold, V., Sjoberg, M., Keane, T. M., Verma, A., Ala, U., et al. (2015). The BRAF pseudogene functions as a competitive endogenous RNA and induces lymphoma in vivo. Cell 161, 319-332.
Krivtsov, A. V., and Armstrong, S. A. (2007). MLL translocations, histone modifications and leukaemia stem-cell development. Nat Rev Cancer 7, 823-833.
Langmead, B., Trapnell, C., Pop, M., and Salzberg, S. L. (2009). Ultrafast and memory-efficient alignment of short DNA sequences to the human genome. Genome Biol 10, R25.
Lanotte, M., Martin-Thouvenin, V., Najman, S., Balerini, P., Valensi, F., and Berger, R. (1991). NB4, a maturation inducible cell line with t(15;17) marker isolated from a human acute promyelocytic leukemia (M3). Blood 77, 1080-1086.
Lengfelder, E., Hofmann, W. K., and Nowak, D. (2012). Impact of arsenic trioxide in the treatment of acute promyelocytic leukemia. Leukemia 26, 433-442.
Li, F., Zhang, L., Li, W., Deng, J., Zheng, J., An, M., Lu, J., and Zhou, Y. (2015a). Circular RNA ITCH has inhibitory effect on ESCC by suppressing the Wnt/beta-catenin pathway. Oncotarget 6, 6001-6013.
Li, P., Chen, S., Chen, H., Mo, X., Li, T., Shao, Y., Xiao, B., and Guo, J. (2015b). Using circular RNA as a novel type of biomarker in the screening of gastric cancer. Clin Chim Acta 444, 132-136.
Li, Y., Zheng, Q., Bao, C., Li, S., Guo, W., Zhao, J., Chen, D., Gu, J., He, X., and Huang, S. (2015c). Circular RNA is enriched and stable in exosomes: a promising biomarker for cancer diagnosis. Cell Res 25, 981-984.
Li, Z., Huang, C., Bao, C., Chen, L., Lin, M., Wang, X., Zhong, G., Yu, B., Hu, W., Dai, L., et al. (2015d). Exon-intron circular RNAs regulate transcription in the nucleus. Nat Struct Mol Biol 22, 256-264.
Liang, D., and Wilusz, J. E. (2014). Short intronic repeat sequences facilitate circular RNA production. Genes Dev 28, 2233-2247.
Lowenberg, B., Pabst, T., Vellenga, E., van Putten, W., Schouten, H. C., Graux, C., Ferrant, A., Sonneveld, P., Biemond, B. J., Gratwohl, A., et al. (2011). Cytarabine dose for acute myeloid leukemia. N Engl J Med 364, 1027-1036.
Lozzio, B. B., and Lozzio, C. B. (1979). Properties and usefulness of the original K-562 human myelogenous leukemia cell line. Leuk Res 3, 363-370.
Martelli, M. P., Sozzi, G., Hernandez, L., Pettirossi, V., Navarro, A., Conte, D.,
Gasparini, P., Perrone, F., Modena, P., Pastorino, U., et al. (2009). EML4-ALK rearrangement in non-small cell lung cancer and non-tumor lung tissues. Am J Pathol 174, 661-670.
Memczak, S., Jens, M., Elefsinioti, A., Torti, F., Krueger, J., Rybak, A., Maier, L., Mackowiak, S. D., Gregersen, L. H., Munschauer, M., et al. (2013). Circular RNAs are a large class of animal RNAs with regulatory potency. Nature 495, 333-338.
Meyerson, M., Gabriel, S., and Getz, G. (2010). Advances in understanding cancer genomes through second-generation sequencing. Nat Rev Genet 11, 685-696.
Qu, S., Yang, X., Li, X., Wang, J., Gao, Y., Shang, R., Sun, W., Dou, K., and Li, H. (2015). Circular RNA: A new star of noncoding RNAs. Cancer Lett 365, 141-148.
Rabbitts, T. H. (1994). Chromosomal translocations in human cancer. Nature 372, 143-149.
Ryan, B. M., Robles, A. I., and Harris, C. C. (2010). Genetic variation in microRNA networks: the implications for cancer research. Nat Rev Cancer 10, 389-402.
Salzman, J, Chen, R. E., Olsen, M. N., Wang, P. L., and Brown, P. O. (2013). Cell-type specific features of circular RNA expression. PLoS Genet 9, e1003777.
Salzman, J, Gawad, C., Wang, P. L., Lacayo, N., and Brown, P. O. (2012). Circular RNAs are the predominant transcript isoform from hundreds of human genes in diverse cell types. PLoS One 7, e30733.
Smith, M. A., Morton, C. L., Phelps, D., Girtman, K., Neale, G., and Houghton, P. J. (2008). SK-NEP-1 and Rhl are Ewing family tumor lines. Pediatr Blood Cancer 50, 703-706.
Somervaille, T. C., and Cleary, M. L. (2010). Grist for the MLL: how do MLL oncogenic fusion proteins generate leukemia stem cells? Int J Hematol 91, 735-741.
Tsuchiya, S., Yamabe, M., Yamaguchi, Y., Kobayashi, Y., Konno, T., and Tada, K. (1980). Establishment and characterization of a human acute monocytic leukemia cell line (THP-1). Int J Cancer 26, 171-176.
Wilks, C., Cline, M. S., Weiler, E., Diehkans, M., Craft, B., Martin, C., Murphy, D., Pierce, H., Black, J., Nelson, D., et al. (2014). The Cancer Genomics Hub (CGHub): overcoming cancer through the power of torrential data. Database (Oxford) 2014.
You, X., Vlatkovic, I., Babic, A., Will, T., Epstein, I., Tushev, G., Akbalik, G., Wang, M., Glock, C., Quedenau, C., et al. (2015). Neural circular RNAs are derived from synaptic genes and regulated by development and plasticity. Nat Neurosci 18, 603-610.
Zahreddine, H., and Borden, K. L. (2013). Mechanisms and insights into drug resistance in cancer. Front Pharmacol 4, 28.
Zhang, X. O., Wang, H. B., Zhang, Y., Lu, X., Chen, L. L., and Yang, L. (2014). Complementary sequence-mediated exon circularization. Cell 159, 134-147.
Zuber, J., Radtke, I., Pardee, T. S., Zhao, Z., Rappaport, A. R., Luo, W., McCurrach, M. E., Yang, M. M., Dolan, M. E., Kogan, S. C., et al. (2009). Mouse models of human AML accurately predict chemotherapy response. Genes Dev 23, 877-889.

1. An isolated f-circRNA or a complement thereof encoding one or more exons or exon fragments from a first gene, one or more exons or exon fragments from a second gene and an f-circRNA back-splice junction.

2. The f-circRNA of claim 1, wherein the first and second genes are arranged as a translocation that:
corresponds to a disorder selected from the group consisting of cancer, translocation Down syndrome, XX male syndrome and schizophrenia;
is selected from the group consisting of a balanced translocation, an unbalanced translocation, a Robertsonian translocation and an insertional translocation; and/or
corresponds to a cancer-associated chromosomal translocation.

3-4. (canceled)

5. The f-circRNA of claim 1, wherein the cancer-associated chromosomal translocation is selected from the group consisting of a PML/RARα translocation, an MLL/AF9 translocation, an EWSR1-FL11 translocation and an AML4/ALK1 translocation wherein the translocation optionally comprises one or any combination of:
a) a PML/RARα translocation comprising a back-splice junction between a 5′ head of PML exon 5 and a 3′ tail of RARα exon 6;
b) a PML/RARα translocation comprising a back-splice junction between a 5′ head of PML exon 4 and a 3′ tail of RARα exon 4;
c) an MLL/AF9 translocation comprising a back-splice junction between a 5′ head of MLL exon 7 and a 3′ tail of AF9 exon 6;
d) an MLL/AF9 translocation comprising a back-splice junction between a 5′ head of MLL exon 5 and a 3′ tail of AF9 exon 6;
e) an EWSR1/FLI1 translocation comprising a back-splice junction between a 5′ head of EWSR1 exon 7 and a 3′ tail of FLI1 exon 10; and
f) an EML4/ALK1 translocation comprising a back-splice junction between a 5′ head of EML4 exon 12 and a 3′ tail of ALK1 exon 26.

6-11. (canceled)

12. The f-circRNA of claim 5, comprising:
a nucleotide sequence having at least 80% homology to a sequence selected from the group consisting of SEQ ID NO:1, SEQ ID NO:2, SEQ ID NO:3, SEQ ID NO:4, SEQ ID NO:5 and SEQ ID NO:6; or
a nucleotide sequence selected from the group consisting of SEQ ID NO:1, SEQ ID NO:2, SEQ ID NO:3, SEQ ID NO:4, SEQ ID NO:5 and SEQ ID NO:6.

13. (canceled)

14. A vector expressing an f-circRNA or a complement thereof, or
an isolated cell expressing an exogenous f-circRNA, or
a cancer model comprising a non-human organism expressing an exogenous f-circRNA.

15. A compound that binds to an f-circRNA back-splice junction of an f-circRNA and inhibits one or more activities of the f-circRNA.

16. The compound of claim 15, comprising an RNA sequence selected from the group consisting of antisense RNA, siRNA and shRNA, optionally wherein:
the RNA sequence inhibits an f-circRNA activity that is optionally selected from the group consisting of cellular proliferation, cellular transformation and carcinogenesis;
the RNA sequence is chemically modified; and/or
the RNA sequence mediates degradation of the f-circRNA.

17-20. (canceled)

21. A method of diagnosing a subject with a translocation-associated disorder or disease comprising detecting in the subject the presence of a fusion-circular RNA (f-circRNA).

22. The method of claim 21, wherein the disorder or disease is a cancer optionally selected from the group consisting of acute promyelocytic leukemia (APL), MLL-AF9-mediated leukemia, Ewing sarcoma and non-small cell lung carcinoma and/or wherein the f-circRNA is detected in a lysosomal fraction obtained from the subject.

23-24. (canceled)

25. A method for treating a translocation-associated disorder or disease in a subject in need thereof comprising contacting the subject with a therapeutic amount of an f-circRNA inhibiting agent effective to reduce one or more symptoms of the translocation-associated disorder or disease in the subject.

26. The method of claim 25, wherein the f-circRNA inhibiting agent targets an f-circRNA back-splice junction of an f-circRNA, optionally wherein:
the agent is selected from the group consisting of antisense RNA, siRNA and shRNA; and/or
the disorder is cancer, optionally selected from the group consisting of APL, MLL-AF9-mediated leukemia, Ewing sarcoma and non-small cell lung carcinoma.

27-29. (canceled)

30. A method selected from the group consisting of:
inducing apoptosis in a cancer cell comprising contacting the cancer cell with a compound that inhibits an f-circRNA activity;
treating cancer in a subject comprising administering to the subject a compound that inhibits an f-circRNA activity;
increasing efficacy of a conventional anti-cancer therapy in a subject comprising contacting a subject receiving conventional anti-cancer therapy with a compound that inhibits an f-circRNA activity in the subject, optionally wherein the conventional anti-cancer therapy is one or both of chemotherapy and radiation;
reducing relapse from remission in a subject comprising administering to the subject a compound that inhibits an f-circRNA activity in the subject;
decreasing proliferation of a cell comprising contacting the cell with a compound that inhibits an f-circRNA activity in the subject; and
prognosing or diagnosing cancer in a subject comprising detecting f-circRNA in an exosome of the subject.

31. The method of claim 30, wherein the compound binds to an f-circRNA back-splice junction of an f-circRNA and is optionally exogenous RNA selected from the group consisting of antisense RNA, siRNA and shRNA.

32-50. (canceled)

51. A method of increasing proliferation rate in a cell comprising contacting the cell with an exogenous f-circRNA, or a method of transforming a cell comprising contacting the cell with an exogenous f-circRNA.

52-53. (canceled)
